SEQUENCE LISTING (29 sequences)

SEQ ID NO: 1 - protein, 4 aa, Artificial Sequence
  Description of Artificial Sequence; note = synthetic construct
KKTK

SEQ ID NO: 2 - protein, 3 aa, Artificial Sequence
  Description of Artificial Sequence; note = synthetic construct
RGD

SEQ ID NO: 3 - protein, 5 aa, Artificial Sequence
  Description of Artificial Sequence; note = synthetic construct
GGGGS

SEQ ID NO: 4 - protein, 14 aa, Artificial Sequence
  Description of Artificial Sequence; note = synthetic construct
DGDITWDQLWDLMK

SEQ ID NO: 5 - DNA, 36029 bp, Artificial Sequence
  Description of Artificial Sequence; note = synthetic construct
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcactctaggcggccgcgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgagctatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatccggtcgcgcgaattgatccaaatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaatcaaatacaaaggatatcaggtggcccccgctgaattggagtcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaaaatgtaactgtattcagcgatgacgaaattcttagctattgtaatcctccgaggcctcgacctgcaggcatgcaagcttgggatctttgtgaaggaaccttacttctgtggtgtgacataattggacaaactacctacagagatttaaagctctaaggtaaatataaaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgcggccgcctagagggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 6 - DNA, 720 bp, Artificial Sequence
  Description of Artificial Sequence; note = synthetic construct
  misc_feature 693,709: n = g, a, c or t(u)
ttcaactaggtgtcctcggatcccacgaagtgaaaattaaacacttttctccgtatcacgaagtgaaaattaaacacttttctccgtatggatcccatcaccatcaccatcacctaggttcacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggntacttttctatgtggnatcaggctggt

SEQ ID NO: 7 - DNA, 719 bp, Artificial Sequence
  Description of Artificial Sequence; note = synthetic construct
  misc_feature 2,3,4,10,12,16,17,42,715,719: n = g, a, c or t(u)
cnnnggaggncnttcnnataggtgtcgaaggtcaaacacctnaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactctcggatcccacgaagtgaaaattaaacacttttctccgtatcacgaagtgaaaattaaacacttttctccgtatggatcccatcaccatcaccatcacctaggttcattgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgntgan

SEQ ID NO: 8 - DNA, 108 bp, Artificial Sequence
  Description of Artificial Sequence; note = synthetic construct
ctcggatcccacgaagtgaaaattaaacacttttctccgtatcacgaagtgaaaattaaacacttttctccgtatggatcccatcaccatcaccatcacctaggttca

SEQ ID NO: 9 - protein, 36 aa, Artificial Sequence
  Description of Artificial Sequence; note = synthetic construct
LGSHEVKIKHFSPYHEVKIKHFSPYGSHHHHHHLGS

SEQ ID NO: 10 - protein, 11 aa, Artificial Sequence
  Description of Artificial Sequence; note = synthetic construct
HEVKIKHFSPY

SEQ ID NO: 11 - protein, 5 aa, Artificial Sequence
  Description of Artificial Sequence; note = synthetic construct
GGGGS

SEQ ID NO: 12 - protein, 12 aa, Artificial Sequence
  Description of Artificial Sequence; note = synthetic construct
LGSHHHHHHLGS

SEQ ID NO: 13 - protein, 3 aa, Artificial Sequence
  Description of Artificial Sequence; note = synthetic construct
KGS

SEQ ID NO: 14 - DNA, 22 bp, Artificial Sequence
  Description of Artificial Sequence; note = synthetic construct
cctacgcacgacgtgaccacag

SEQ ID NO: 15 - DNA, 62 bp, Artificial Sequence
  Description of Artificial Sequence; note = synthetic construct
tgaacctaggtgatggtgatggtgatgggatccgaggacacctatttgaataccctcctttg

SEQ ID NO: 16 - DNA, 61 bp, Artificial Sequence
  Description of Artificial Sequence; note = synthetic construct
ctcggatcccatcaccatcaccatcacctaggttcacctaaatatgccgataaaacatttc

SEQ ID NO: 17 - DNA, 23 bp, Artificial Sequence
  Description of Artificial Sequence; note = synthetic construct
ctagggagctctgcagaaccatg

SEQ ID NO: 18 - DNA, 60 bp, Artificial Sequence
  Description of Artificial Sequence; note = synthetic construct
tgaacctaggtgatggtgatggtgatgggatccgagttcgtaccactgagattctcctat

SEQ ID NO: 19 - DNA, 60 bp, Artificial Sequence
  Description of Artificial Sequence; note = synthetic construct
ctcggatcccatcaccatcaccatcacctaggttcaactgaaattaatcatgcagctggg

SEQ ID NO: 20 - DNA, 61 bp, Artificial Sequence
  Description of Artificial Sequence; note = synthetic construct
tgaacctaggtgatggtgatggtgatgggatccgagagtagttgagaaaaattgcatttcc

SEQ ID NO: 21 - DNA, 60 bp, Artificial Sequence
  Description of Artificial Sequence; note = synthetic construct
ctcggatcccatcaccatcaccatcacctaggttcattgactcctaaagtggtattgtac

SEQ ID NO: 22 - DNA, 61 bp, Artificial Sequence
  Description of Artificial Sequence; note = synthetic construct
tgaacctaggtgatggtgatggtgatgggatccgagagtgggcatgtaagaaatatgagtg

SEQ ID NO: 23 - DNA, 58 bp, Artificial Sequence
  Description of Artificial Sequence; note = synthetic construct
ctcggatcccatcaccatcaccatcacctaggttcaaactcacgagaactaatgggcc

SEQ ID NO: 24 - DNA, 60 bp, Artificial Sequence
  Description of Artificial Sequence; note = synthetic construct
tgaacctaggtgatggtgatggtgatgggatccgagaggttttaccttggtaagagtctc

SEQ ID NO: 25 - DNA, 62 bp, Artificial Sequence
  Description of Artificial Sequence; note = synthetic construct
ctcggatcccatcaccatcaccatcacctaggttcatgggaaaaagatgctacagaattttc

SEQ ID NO: 26 - DNA, 60 bp, Artificial Sequence
  Description of Artificial Sequence; note = synthetic construct
tgaacctaggtgatggtgatggtgatgggatccgagtggaaagcagtaatttggaagttc

SEQ ID NO: 27 - DNA, 63 bp, Artificial Sequence
  Description of Artificial Sequence; note = synthetic construct
ctcggatcccatcaccatcaccatcacctaggttcaaataattttgccatggaaatcaatcta

SEQ ID NO: 28 - DNA, 720 bp, Artificial Sequence
  Description of Artificial Sequence; note = synthetic construct
  misc_feature 1-14, 123-720: n = g, a, c or t(u)
nnnnnnnnnnnnnnctcggatcccacgaagtgaaaattaaacacttttctccgtatcacgaagtgaaaattaaacacttttctccgtatggatcccatcaccatcaccatcacctaggttcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 29 - DNA, 720 bp, Artificial Sequence
  Description of Artificial Sequence; note = synthetic construct
  misc_feature 1-282, 390-720: n = g, a, c or t(u)
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctcggatcccacgaagtgaaaattaaacacttttctccgtatcacgaagtgaaaattaaacacttttctccgtatggatcccatcaccatcaccatcacctaggttcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

(19) United States
(12) Patent Application Publication
(10) Pub. No.: US 2007/0036721 A1
(43) Pub. Date: Feb. 15, 2007

(54) Methods and compositions for in vivo inflammation monitoring

(75) Inventors: Kurt R. Zinn, Hoover, AL (US); Tandra R. Chaudhuri, Hoover, AL (US); Hongju Wu, Birmingham, AL (US)

Correspondence Address: NEEDLE & ROSENBERG, P.C., SUITE 1000, 999 PEACHTREE STREET, ATLANTA, GA, 30309-3915, US

(73) Assignee: UAB RESARCH FOUNDATION, Birmingham, AL (US)

(21) Appl. No.: 10/572,732

(22) PCT Filed: Sep. 23, 2004
(86) PCT No.: PCT/US04/31141

§ 371 Date: Jul. 18, 2006

Related U.S. Application Data
(60) Provisional application No. 60/505,543, filed on Sep. 23, 2003.

(51) Int. Cl. A61K 49/00; C12Q 1/70; C12Q 1/68; C07H 21/04; C12N 15/86

(52) U.S. Cl. 424/9.1; 435/6; 435/5; 435/456; 536/23.2

(57) ABSTRACT

Disclosed are methods and materials for detecting inflammation using in vivo and in vitro monitoring, as well as methods of reducing inflammation. Also disclosed are composition related to in vivo monitoring of inflammation, as well as transgenic animals and cell lines useful for such.

This application claims priority to U.S. Provisional Application No. 60/505,543 filed Sep. 23, 2003.

This invention was made with government support under grant number CA80104 awarded by the U.S. National Institute of Health. The United States Government has certain rights in this invention.

### I. BACKGROUND OF THE INVENTION

A. Field of the Invention

This invention relates generally to methods of monitoring inflammation. This invention also relates to methods of identifying a vector capable of detecting inflammation. The invention further relates to methods of treating inflammatory disease. This invention also relates to cell lines and transgenic animals useful for monitoring inflammation. The invention has broad applicability in medicine as a method of identifying and treating diseases and disorders related to inflammation.

B. Background Art

Noninvasive monitoring of light emitted from within a living mammal, or molecular imaging where the light is constitutively expressed by a reporting gene, provides an opportunity for obtaining specific information about physiological processes and whole biological systems. Molecular imaging is important in the evaluation of therapeutic approaches for genetic diseases. Molecular imaging offers advantages for the evaluation of new molecular therapies, including gene therapy. Imaging can confirm in vivo targeting or it can be used to monitor molecular responses induced by therapy. For gene therapy approaches, the extent and magnitude of both gene transfer and expression can be determined by molecular imaging.

Reporter genes with optical signatures (e.g. fluorescence, color or bioluminescence) have been used in cell culture, in small organisms that are relatively transparent (Drosophila) or two dimensional (plant leaves), and in ex vivo analyses after expression in larger animals. In such assays reporter genes are linked to genetic regulatory elements and can reveal spatial and temporal information about a variety of biological processes at the level of transcription. What is needed in the art is a method of monitoring in vivo biological processes, such as inflammation, in subjects such as animals and humans.

Inflammation is a complex stereotypical reaction of the body expressing the response to damage of its cells and vascularized tissues. The discovery of the detailed processes of inflammation has revealed a close relationship between inflammation and the immune response. The main features of the inflammatory response are vasodilation, i.e. widening of the blood vessels to increase the blood flow to the infected area; increased vascular permeability, which allows diffusible components to enter the site; cellular infiltration by chemotaxis, or the directed movement of inflammatory cells through the walls of blood vessels into the site of injury, changes in biosynthetic, metabolic, and catabolic profiles of many organs; and activation of cells of the immune system as well as of complex enzymatic systems of blood plasma.

There are two forms of inflammation, acute and chronic. Acute inflammation can be divided into several phases. The earliest, gross event of an inflammatory response is temporary vasoconstriction, i.e. narrowing of blood vessels caused by contraction of smooth muscle in the vessel walls, which can be seen as blanching (whitening) of the skin. This is followed by several phases that occur over minutes, hours and days later. The first is the acute vascular response, which follows within seconds of the tissue injury and lasts for several minutes. This results from vasodilation and increased capillary permeability due to alterations in the vascular endothelium, which leads to increased blood flow (hyperemia) that causes redness (erythema) and the entry of fluid into the tissues (edema).

This can be followed by an acute cellular response, which takes place over the next few hours. The hallmark of this phase is the appearance of granulocytes, particularly neutrophils, in the tissues. These cells first attach themselves to the endothelial cells within the blood vessels (margination) and then cross into the surrounding tissue (diapedesis). During this phase erythrocytes may also leak into the tissues and a hemorrhage can occur. If the vessel is damaged, fibrinogen and fibronectin are deposited at the site of injury, platelets aggregate and become activated, and the red cells stack together in what are called “rouleau” to help stop bleeding and aid clot formation. The dead and dying cells contribute to pus formation. If the damage is sufficiently severe, a chronic cellular response may follow over the next few days. A characteristic of this phase of inflammation is the appearance of a mononuclear cell infiltrate composed of macrophages and lymphocytes. The macrophages are involved in microbial killing, in clearing up cellular and tissue debris, and in remodeling of tissues.

Chronic inflammation is an inflammatory response of prolonged duration—weeks, months, or even indefinitely—whose extended time course is provoked by persistence of the causative stimulus to inflammation in the tissue. The inflammatory process inevitably causes tissue damage and is accompanied by simultaneous attempts at healing and repair. The exact nature, extent and time course of chronic inflammation is variable, and depends on a balance between the causative agent and the attempts of the body to remove it. Etiological agents producing chronic inflammation include: (i) infectious organisms that can avoid or resist host defenses and so persist in the tissue for a prolonged period. Examples include Mycobacterium tuberculosis, Actinomycetes, and numerous fungi, protozoa and metazoal parasites. Such organisms are in general able to avoid phagocytosis or survive within phagocytic cells, and tend not to produce toxins causing acute tissue damage. (ii) Infectious organisms that are not innately resistant but persist in damaged regions where they are protected from host defenses. An example is bacteria which grow in the pus within an undrained abscess cavity, where they are protected both from host immunity and from blood-borne therapeutic agents, e.g. antibiotics. Some locations are particularly prone to chronic abscess formation, e.g. bone, and pleural cavities. (iii) Irritant non-living foreign material that cannot be removed by enzymatic breakdown or phagocytosis. Examples include a wide range of materials implanted into wounds (wood splinters, grit, metals and plastics), inhaled (silica dust and other particles or fibers), or deliberately introduced (surgical prostheses, sutures, etc.) Also included are transplants. Dead tissue components that cannot be broken down may have similar effects, e.g. keratin squames from a ruptured epidermoid cyst or fragments of dead bone (sequestrum) in osteomyelitis. (iv) In some cases the stimulus to chronic inflammation may be a normal tissue component. This occurs in inflammatory diseases where the disease process is initiated and maintained because of an abnormality in the regulation of the body's immune response to its own tissues—the so-called auto-immune diseases. (v) For many diseases characterized by a chronic inflammatory pathological process the underlying cause remains unknown. A good example is Crohn's disease of the intestine.

Examples of chronic inflammatory diseases include tuberculosis, chronic cholecystitis, bronchiectasis, rheumatoid arthritis, Hashimoto's thyroiditis, inflammatory bowel disease (ulcerative colitis and Crohn's disease), silicosis and other pneumoconiosis, and implanted foreign body in a wound.

Activation of innate immunity and promotion of inflammation are common responses to replication incompetent adenoviruses (Ad) now being developed as vectors for gene therapy (Jooss, K. (2003) Gene Ther. 10:955-963; Zaiss, A. K. (2002) J. Virol. 76:4580-4590, Rux et al. (2000) Mol Ther 1:18-30; Rux et al. (2003) J. Virol. 77:9553-9556). This is a major obstacle to the use of adenovirus as a vector for gene therapy. Needed in the art are vectors with modified or chimeric hexons to evade the immune response to native hexon. The hexon is a structural protein of the Ad capsid; there are a total of 240 trimeric hexon proteins in each Ad capsid. There are seven hypervariable regions (HVRs) of the Ad hexon for each subunit of the trimer, the HVRs contain serotype-specific residues. Insertion of a specific residue in the HVR region results in 240×3, or 720 total inserts per Ad vector.

The complement system is central to both innate immunity and inflammation (Walport, M. J. (2001) N Eng J Med 344:1058-1066 and 1140-1144). Because it is comprised of multiple membrane-bound and blood factors, the complement system is of particular relevance in delivery of vectors administered intravenously. In fact, Cichon et al. showed complement was activated in a majority of human plasma samples when challenged with different adenoviral serotypes; complement activation was completely dependent on anti-Ad antibody (Cichon (2001) Gene Ther 8:1794-1800).

The complement mediated inactivation is a multistep enzymatic cascade which finally results in formation of a membrane attack complex (MAC) mediating the perforation of membranes and subsequent lysis of the invading organism. It is either initiated by antigen-antibody complexes (classical pathway) or via an antibody independent pathway which is activated by certain particular polysaccharides, viruses and bacteria (alternative pathway).

Human organs and cells themselves are protected to complement mediated lysis. This protection is achieved by expression of complement inactivation factors. So far, five human factors are known. CD35 (CR1) is released from the cells and acts mainly extrinsically. In contrast, CD59, CD46 (MCP), CD55 (DAF) and HRF are integrated into the cellular membrane. CD46 (MCP) is a classical transmembrane protein while HRF, CD59 and CD55 are GPI-anchored. These factors can interrupt the complement cascade at two different stages: DAF, CR1 and MCP act at an early stage of both the alternative and the classical pathway. In contrast, CD59 and HRF inhibit the assembly of the membrane attack complex, which is the final step of both pathways resulting in channel formation and lysis.

What is needed in the art is a method of monitoring inflammation in vivo. Also needed is a method of utilizing the complement system to enhance inflammation monitoring or to reduce inflammation in subjects.

### II. SUMMARY OF THE INVENTION

In accordance with the purpose(s) of this invention, as embodied and broadly described herein, this invention, in one aspect, relates to a method of detecting inflammation in a subject by in vivo monitoring. More specifically, the method comprises administering to a subject a vector, the vector comprising a reporter nucleic acid operably linked to a promoter nucleic acid, wherein said reporter nucleic acid is expressed under conditions of inflammation, and detecting expression of said reporter nucleic acid by in vivo monitoring.

In another aspect, the invention relates to a method of detecting inflammation in a transplant recipient. More specifically the method comprises administering to cells of the transplant, prior to transplantation, a vector, said vector comprising a reporter nucleic acid and a promoter nucleic acid, wherein expression of the reporter nucleic acid is detectable under conditions of inflammation; performing the transplant; and detecting expression of the reporter nucleic acid by in vivo monitoring.

The invention also relates to a method of monitoring inflammation in a subject with an inflammatory or autoimmune disease.

In yet another aspect, the invention relates to a method of identifying a vector capable of detecting inflammation. Specifically, the method comprises administering a vector to a cell culture, wherein the vector comprises a promoter nucleic acid and a reporter nucleic acid; inducing an inflammatory response in said cell culture; and monitoring expression of the reporter nucleic acid, expression indicating a vector capable of detecting inflammation.

The invention also relates to a method of treating a subject with an inflammatory disease. Specifically, the method comprises administering to a subject a vector, the vector comprising a reporter nucleic acid operably linked to a promoter nucleic acid, wherein the reporter nucleic acid is expressed under conditions of inflammation; detecting expression of said reporter nucleic acid by in vivo monitoring; and modifying treatment of the subject when expression of said reporter nucleic acid is detected.

In another aspect, the invention relates to a method of reducing inflammation in a subject, comprising delivering to the subject a complement modulator.

In yet another aspect, the invention relates to a composition comprising a transgenic animal, wherein the animal comprises a reporter nucleic acid operably linked to a promoter nucleic acid, wherein the reporter nucleic acid is expressed under conditions of inflammation.

In another aspect, the invention relates to a composition comprising a cell line that comprises a vector, the vector comprising a reporter nucleic acid operably linked to a promoter nucleic acid, wherein the reporter nucleic acid is expressed under conditions of inflammation.

The invention offers distinct advantages over the prior art because disclosed are methods for in vivo monitoring of inflammation, as well as methods of reducing inflammation (including reducing cellular and humoral immune responses) in a subject, and transgenic animals and cell lines. Additional advantages of the invention will be set forth in part in the description which follows or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention as claimed.

### III. BRIEF DESCRIPTION OF THE DRAWINGS

The accompanying drawings, which are incorporated in and constitute a part of this specification, illustrate embodiments of the invention and together with the description, serve to explain the principles of the invention.

FIG. 1 shows overlays of mice images with pseudocolor images; in which the different colors represent the intensity of light emission from the mouse. The relative photons emitted in an area of the mouse were determined by region of interest analyses. For all mice, the luciferase expression in liver was extremely low, essentially undetectable by 3 days after dosing with Ad-cox2L-Luc (FIG. 1A). At 10 minutes after injection of 2 micrograms of LPS, no induction of luciferase expression was detectable. However, by 4 hr after injection of LPS the induced luciferase expression was detected by imaging approximately 12-fold over background signal (FIG. 1C-D). The expression was transient, and was reduced to nearly background signal by 24 h after LPS injection (FIG. 1E-F).

FIG. 2 shows that luciferase expression in lung was detected at 10 days after intratracheal (i.t.) delivery of an Ad encoding luciferase (driven by CMV, 5×10⁸ pfu). Two views are shown in FIG. 2, demonstrating the capacity of the bioluminescence system for detection of lung luciferase expression.

FIG. 3 shows the emission SPECT images accurately fused with the anatomic CT image. In this example the mouse was i.v. injected with Tc-99m-labeled macroaggregated albumin (MAA, 300 microcuries) microspheres that are trapped in the capillaries of the lung. The SPECT imaging session required ˜30 minutes to acquire 64 views. As shown, the fusion images accurately reveal the expected distribution of the Tc-99m-MAA throughout the entire lung. The SPECT/CT fusion is important for accurately determining the location of Tc-99m-labeled radiotracers at 1-mm resolution in the mouse, for example, to determine the precise location of Tc-99m-labeled Ad vectors, or hSSTr2 transgene expression by imaging specific retention of the Tc-99m-labeled hSSTr2-avid peptide. SPECT imaging is commonly applied for human imaging applications.

FIG. 4 shows bioluminescence imaging of luciferase expression in living mice at 13 days after intravenous injection of 2.3×10⁹ v.p. of Ad5Luc1 in (A) wild type control mice, and (B) C3^{-/-} mice. The pseudocolor overlay represents the intensity of light emission, and thus the level of luciferase expression. A single lot of E1-deleted recombinant Ad5Luc1 containing the firefly luciferase gene under control of CMV promoter was used; all injections of the virus were intravenous. Studies with a range of Ad5Luc1 doses (2×10⁹-1×10¹⁰ v.p.) showed from 10- to 100-fold less expression of luciferase in C3^{-/-} mice (4 mice) versus matched controls (4 mice). Based on these initial studies, three additional experiments each with 2 groups of mice each (control and C3^{-/-} mice, n=3-4/group) were conducted to evaluate 3 different Ad5Luc1 doses (2.3×10⁹, 4.0×10⁹, and 1.3×10¹⁰ v.p.). At various times after administration of Ad5Luc1, the mice were imaged using a bioluminescence imaging system (Xenogen, Inc.) to detect luciferase expression. Images were collected on mice oriented in the same position and always 10 min after intraperitoneal injection of 2.5 mg luciferin. The mice were maintained under enflurane anesthesia at 37° C., with their ventral surfaces facing the CCD camera that was part of the imaging system. Imaging was performed several times on each mouse, beginning at 6 hr after Ad5Luc1 injection and continuing to day 34. Data acquisition times for imaging ranged from 20 sec to 10 min.

FIG. 5 shows liver light emission (luciferase expression) over time in 3 experiments. Mice were intravenously dosed with (A) 2.3×10⁹ v.p./mouse, (3) 4.0×10⁹ v.p./mouse, and (C) 1.3×10¹⁰ v.p./mouse. The numbers adjacent to the wild type data points indicate the fold greater expression for the wild type group relative to the C3^{-/-} group for that time point, with the “*” indicating statistical significance at p<0.05. Each line is representative of 4 mice, except there were only 3 control mice in (3). Male mice (A) and female mice (B-C) were used. Light emission from the liver region (relative photons/sec) was measured using software provided by Xenogen, and the intensity represents the liver luciferase activity. This relationship was validated by comparing luciferase measurements from the live animals with independent measurements obtained from tissue homogenates. These comparisons were accomplished at termination by removal of liver and spleen (mice injected with 2.3×10⁹ v.p.), followed by independent in vitro luciferase analyses as described. The validation also confirmed that the liver was responsible for >99% of the light emission that was detected in the liver region of the live mice using the Xenogen system.

FIG. 6 shows imaging uptake of a Tc-99m-labeled hSSTr2-avid peptide (P2045) in a subcutaneous xenograft by (A) planar gamma camera imaging, and (B) combined SPECT/CT imaging. The hSSTr2 expression was induced in the A-427 xenograft in the mouse by direct injection of the tumor 48 h earlier with a replication incompetent Ad vector encoding hSSTr2. The Tc-99m-P2045 was injected intravenously (1 mCi). Images were collected at 5 h after injection of Tc-99m-P2045. The SPECT/CT allowed easy visualization of tumor, and excluded activity in the intestines, kidneys, and bladder. The SPECT image was automatically collected in 34 min (64 views, 30 s each) while the CT image was collected in 5 min (256 views, 0.5 s each). The mouse was anesthetized and in the same position for both SPECT and CT imaging procedures.

FIG. 7 shows in vivo imaging of luciferase expression in A-427 subcutaneous xenograft tumors. (A) ventral image, (B) dorsal image. Mice #1, 2, and 3 were i.v. injected with a replication incompetent Ad encoding luciferase (4 d earlier). Mouse #4 did not receive an Ad injection. Black arrows indicate the luciferase expression in the tumors.

FIG. 8 shows bioluminescence imaging of luciferase expression in a subcutaneous (s.c.) prostate (PC3) tumor following i.v. injection of a replication competent Ad vector encoding luciferase (Ad5Luc3). The same mice were imaged at (A) 14 d and (B) 21 d after i.v. injection of the Ad vector. The control images on the right (A-B) were from a nude mouse bearing s.c. tumors, without an i.v. Ad injection.

FIG. 9 shows dual light-based imaging of GFP-positive intraperitoneal prostate (PC3) tumors following i.v. injection of a replication competent Ad vector encoding luciferase (Ad5Luc3). The same mice were imaged for luciferase on (A) 7 d and (B) 28 d after i.v. injection of the Ad vector; and by fluorescence imaging at 28 d with (C-D) GFP-positive tumors from mouse #1, (E) GFP-positive tumors from mouse #2, and by bright field imaging (F) showing tumors in mouse #2. There was excellent correlation between the GFP-positive tumors in mice #1 and #2 with the luciferase signal induced by i.v. injection of the Ad vector, with detection by luciferase imaging. The two control mice on the right (A-B) were nude mice bearing i.p. tumors, without an i.v. Ad injection. The black rectangles (solid and dashed) in B can be compared with the identical regions in C-E indicated by the white rectangles.

FIG. 10 shows luciferase expression in SKOV3 and OV-4 cells following infection with Ad5Luc1 and Ad5LucFF/CD40L (targeting CD40 receptor). Infection with Ad5Luc1 was blocked with Ad5 knob, but not CD40L. Infection with the Ad5LucFF/CD40L was blocked with CD40L, but not Ad5 knob. There was higher luciferase expression in cells infected with the Ad5LucFF/CD40L relative to cells infected with Ad5Luc1. The fiber-fibroin (FF) construct is designed for insertion of other targeting ligands (like CD40L), with elimination of the native tropism mediated by the wild-type Ad5 fiber.

FIG. 11 shows in vivo imaging of luciferase expression in liver of athymic nude mice (n=10) after i.v. injection of Ad encoding luciferase (normal fiber structure; A#1,A#2,A#3), Ad encoding luciferase (fiber replaced with FF chimera/6His insert=Ad5LucFF/6H; B#1,B#2,B#3), Ad encoding luciferase (fiber replaced with FF chimera/CD40Ligand insert-Ad5LucFF/CD40L, C#1,C#2,C#3,C#4). Mean liver light emission over time for the 3 groups is shown. Mice were i.v. injected with the same dose of viral particles numbers (2.5×10¹⁰ particles) on day 0. Images shown were 20 s image A#2 B#1 C#2 C#1 A#1 collected after 11 d (exposures are indicated). Replacing the fiber structure with the FF chimera (coupled to ligands) led to markedly reduced luciferase expression in the liver. Mice injected with Ad containing FF chimeras were also imaged for longer times to increase the sensitivity for analyses.

FIG. 12 shows Luciferase expression in i.p SKOV3 tumors following i.v. injection (5 d earlier) of replication incompetent (A-B) Ad5LucFF/CD40L (targeting CD40), or (C) Ad5Luc1. The 2 representative mice (A-B) i.v. injected with Ad5LucFF/CD40L showed luciferase expression in the i.p. tumors; the mice i.v. injected with Ad5Luc1 (C, one representative) had no detectable luciferase expression in the i.p. tumors.

FIG. 13 shows in vivo imaging of Tc-99m-labeled somatostatin receptor-avid peptide (P2045) binding to somatostatin receptor-positive mammary tumors induced with a carcinogen (MNU). (A) picture of a rat in position for imaging with tumors indicated by arrows, (B) planar gamma camera image at 5 h after i.v. injection of Tc-99m-P2045, (C) 5 hr image of a second rat with Tc-99m-P2045 in tumors.

FIG. 14 shows in vivo imaging of tumor binding of a Tc-99m-labeled antibody targeting rat tumor endothelium. Arrows indicate retention of the antibody at the tumor sites at 5 h after i.v. injection of Tc-99m-antibody.

FIG. 15 shows in vivo imaging of luciferase expression in liver of (A) C57B/6 control mice, (B) C3 knockout C57B/6 mice, (C) mean liver light emission over time. Mice were i.v. injected with the same dose of replication incompetent Ad5 encoding luciferase (5.0×10⁹ particles) on day 0. Images shown were collected after 10 d (1 min exposures).

FIG. 16 shows mean liver light emission (luciferase) over time in C57B/6 control mice (n=5) and C3 knockout C57B/6 mice (n=5). Mice were i.v. injected with the same dose of a replication incompetent Ad5 encoding luciferase (1.6×10¹⁰ particles) on day 0.

FIG. 17 shows imaging inflammation. (A) shows an absence of luciferase expression in liver at 4 d after Ad-cox2L-luciferase (before LPS), (B) 4 hr after LPS (2 μg), (C) 4 hr after LPS (lateral view), and (D) 24 hr after LPS. Luciferase expression in liver and spleen was increased 12-fold at 4 hr after LPS, returning to baseline by 24 h.

FIG. 18 shows imaging inflammation. (A) shows an absence of luciferase expression in liver at 4 d after Ad-cox2L-luciferase (before hepatitis-inducing Jo2), (B) 24 hr after Jo2 (3 μg), (C) Image at 4 hr after i.v. injection of an irrelevant Ad (3×10⁹ pfu), 48 h after Jo2. The Jo2 dose was very low, increasing luciferase expression in ⅔ mice. The regions of interest measure the photons of light 25 emitted in the liver area Images were 300 s each.

FIG. 19 shows increasing doses of Jo2 antibody (i.v. injected) to induce inflammation. The lowest dose (0.8 μg) (FIG. 19B) produced only mild increases in luciferase expression in ⅔ of the mice. A slightly greater response was noted for the next dose (1.6 μg) (FIGS. 23C and 23D), while the highest dose (3.2 μg) (FIGS. 19E and 19F) resulted in higher luciferase expression in liver by 6 h in ⅔ of the mice, and 24 h. The luciferase expression in liver remained an additional 24 h. One mouse did not show luciferase expression in liver. The 3.2 μg Jo2 dose is not lethal and is considered a mild stress to the liver.

FIG. 20 shows the same mice were injected with an unrelated Ad vector (3×10⁹ pfu) to simulate conditions where a gene therapy vector would be delivered to liver that was previously subjected to a mild inflammatory reaction (i.e. as simulated by Jo2). The unrelated Ad did induce luciferase expression in the liver in ⅔ mice. Of interest, persistent inflammation was detected in the male mouse in liver and testis even 5 days later. In animals not treated previously with Jo2, the unrelated Ad dose did not increase luciferase expression in liver.

FIG. 21 shows mice were injected with the Ad-cox2L-Luc first, then with an unrelated Ad vector. The unrelated Ad did not induce luciferase expression in liver, even after a second dose of unrelated Ad (FIG. 21A-D). However, a low dose of LPS (2 μg) induced luciferase expression in liver and spleen by 4 h after LPS injection (FIGS. 21E and 21F). After 24 h the liver luciferase was reduced.

FIG. 22 shows an example of a method to establish luciferase-positive PC3 cell lines. The method includes two steps. First, a low number of cells (cancer cells or otherwise) are infected with the adeno-associated virus (AAV) encoding luciferase. Next, the infected cells are diluted and transferred to 96-well plates, with the goal of obtaining 1-2 cells per well. After approximately 2 weeks the intact plate with live cells is imaged by the bioluminescence technique. As shown in the example presented in (A), the imaging allows luciferase-positive cells to be identified. The positive clone is then subjected to another round of screening, as shown in (B). In this example there were 95/96 wells that were positive, indicating the high percentage of luciferase-positive cells and efficiency of the technique.

FIG. 23 shows that two groups of mice are equal during the first ten days of dosing when both groups receive 4E10 v.p. of Ad5FF/6His. However, wild type mice eliminate the liver infected cells due to the immune response. This does not happen with the C3 knockout mice. The normal fiber structure is replaced by fibritin in the vector.

FIG. 24 shows that complement facilitates infection of the liver. Two groups of mice both receive 4E9 v.p. of Ad5FF/6His. The normal fiber structure is replaced by fibritin in the vector. The wild type mice initially display higher levels of infection which taper off, while the C3 knockout mice show steady levels of infection with no marked decrease.

FIG. 25 shows SDS-PAGE for purified viruses. The inserts in the Ad vectors resulted in proteins that were the correct size; “GL” refers to GFP and Luciferase, reporters are also included.

FIG. 26 shows liver luciferase expression for Ad5.HVR2.rH17d′ and Ad5.HVR2.6His over 30 days. Each vector was i.v. injected in 6 BL6 mice; the dose=4×10⁹ v.p viral particles (v.p.).

FIG. 27 shows liver luciferase expression for Ad5.HVR2.rH17d′ and Ad5.HVR2.6His. Each vector was i.v. injected in 6 BL/6 mice; the dose=4×10⁹ v.p. (viral particles). Values on the graph are means (+/−SD) for the 6 mice.

FIG. 28 shows liver luciferase expression for Ad5.HVR5.rH17d′ and Ad5.HVR5.6His. Each vector was i.v. injected in 5 BL/6 mice; the dose=4×10⁹ viral particles.

FIG. 29 shows liver luciferase expression for Ad5.HVR5.rH17d′ and Ad5.HVR5.6His. Each vector was i.v. injected in 5 BL/6 mice; the dose=4×10⁹ viral particles Values on the graph are means (+/−SD) for the 5 mice.

FIG. 30 shows IgG antibody levels (using an ELISA plate coated with Ad5.HVR2.rH17d′) for mice injected with Ad5.HVR2.rH17d′, Ad5.HVR2.6His, and control mice without vector injection. Each vector was i.v. injected in 6 BL/6 mice; the dose=4×10⁹ viral particles Sera was collected after 28days from all mice, sera from each group was pooled, with analyses in duplicate of serial 3-fold dilutions.

FIG. 31 shows IgG antibody levels (using an ELISA plate coated with Ad5.HVR2.6His) for mice injected with Ad5.HVR2.rH17d′, Ad5.HVR2.6His and control mice without vector injections. Each vector was i.v. injected in 6 BL/6 mice; the dose=4×10⁹ viral particles. Sera were collected after 28 days from all mice from each group, sera werepooled; and analyses were performed in duplicate of 3-fold dilutions.

FIG. 32 shows IgM antibody levels (using an ELISA plate coated with Ad5.HVR2.rH17d′) for mice injected with Ad5.HVR2.rH17d′, Ad5.HVR2.6His and control mice without vector injections. Each vector was i.v. injected in 6 BL6 mice; the dose=4×10⁹ viral particles. Sera were collected after 28 days from all mice from each group, sera were pooled; and analyses were performed in duplicate of 3-fold dilutions.

FIG. 33 shows IgM antibody levels (using an ELISA plate coated with Ad5.HVR2.6His) for mice injected with Ad5.HVR2.rH17d′, Ad5.HVR2.6His and control mice without vector injections. Each vector was i.v. injected in 6 BL/6 mice; the dose=4×10⁹ viral particles. Sera were collected after 28 days from all mice from each group, sera were pooled; and analyses were performed in duplicate of 3-fold dilutions.

FIG. 34 shows IgG antibody levels (using an ELISA plate coated with Ad5.HVR5.rH17d′) for mice injected with Ad5.HVR5.rH17d′, Ad5.HVR5.6His and control mice without vector injections. Each vector was i.v. injected in 6 BL/6 mice; the dose=4×10⁹ viral particles. Sera were collected after 14 days from all mice from each group, sera were pooled; and analyses were performed in duplicate of 3-fold dilutions.

FIG. 35 shows IgG antibody levels (using an ELISA plate coated with Ad5.HVR5.6His) for mice injected with Ad5.HVR5.rH17d′, Ad5.HVR5.6His and control mice without vector injections. Each vector was i.v. injected in 6 BL/6 mice; the dose=4×10⁹ viral particles. Sera were collected after 14 days from all mice from each group, sera were pooled; and analyses were performed in duplicate of 3-fold dilutions. FIG. 36 shows IgM antibody levels (using an ELISA plate coated with Ad5.HVR5.rH17d′) for mice injected with Ad5.HVR5.rH17d′, Ad5.HVR5.6His and control mice without vector injections. Each vector was i.v. injected in 6 BL/6 mice; the dose=4×10⁹ viral particles. Sera were collected after 14 days from all mice from each group, sera werepooled; and analyses were performed in duplicate of 3-fold dilutions.

FIG. 37 shows IgM antibody levels (using an ELISA plate coated with Ad5.HVR5.6His) for mice injected with Ad5.HVR5.rH17d′, Ad5.HVR5.6His and control mice without vector injections. Each vector was i.v. injected in 6 BL/6 mice; the dose=4×10⁹ viral particles. Sera were collected after 14 days from all mice from each group, sera werepooled; and analyses were performed in duplicate of 3-fold dilutions.

FIG. 38 shows Luc expression in A427 tumors using Ad5.HVR2.rH17d′ and Ad5.HVR2.6His Each vector was injected directly in the s.c. tumor growing in nude mice; Dose1.25×10⁹ v.p.; values on the graph are means (+/−SD) for the seven tumors.

FIG. 39 shows Luc expression in A427 tumors using Ad5.HVR5.rH17d′ and Ad5.HVR5.6His Each vector was injected directly in the s.c. tumor growing in nude mice; Dose=1.25×10⁹ v.p.; values on the graph are means (+/−SD) for the seven tumors.

FIG. 40 shows whole-body images of firefly luciferase expression, including in lungs of Mouse 1, 2, and 3, after Ad-mediated transfer via controlled intratracheal delivery of an Ad5 encoding firefly luciferase and hSSTr2. The Ad5 dose for Mouse 4 was not delivered in the lung, rather the esophagus.

FIG. 41 shows Mice 3-4, with different scaling of the images that depict firefly luciferase expression. Mouse 3 showed high luciferase expression in lung after Ad-mediated transfer via controlled i.t. delivery, while Mouse 4 was negative in lung since it was dosed via the esophagus.

FIG. 42 shows SPECT/CT imaging from the same Mouse 3 (as FIG. 40-41) and another control mouse without Ad5 administration in lung. Mouse 3 showed a high level of hSSTr2 expression in lung as indicated by retention of the hSSTr2-avid Tc-99m-P2045 peptide in the lung region at 5 hours after i.v. delivery of the radiotracer., There was no detectable hSSTr2 lung expression in the control mouse as indicated by no retention of the Tc-99m-P2045 in lung.

FIG. 43 shows sequence alignment data between HVR2-rH17d′, rH17d′-6His, and consensus sequence.

FIG. 44 shows sequence alignment data between HVR5-rH17d′, rH17d′-6His, and consensus sequence

### IV. DETAILED DESCRIPTION OF THE INVENTION

The present invention may be understood more readily by reference to the following detailed description of preferred embodiments of the invention and the Examples included therein and to the Figures and their previous and following description.

A. Definitions

As used in the specification and the appended claims, the singular forms “a,” “an” and “the” include plural referents unless the context clearly dictates otherwise. Thus, for example, reference to “a small molecule” includes mixtures of one or more small molecules, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The terms “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control. For example, basal levels are normal in vivo levels prior to, or in the absence of, inflammation or the addition of an agent which causes inflammation.

“Inflammation” or “inflammatory” is defined as the reaction of living tissues to injury, infection, or irritation. Anything that stimulates an inflammatory response is said to be inflammatory.

“Inflammatory disease” is defined as any disease state associated with inflammation. The inflammation can be associated with an inflammatory disease. Examples of inflammatory disease include, but are not limited to, asthma, systemic lupus erythematosus, rheumatoid arthritis, reactive arthritis, spondyarthritis, systemic vasculitis, insulin dependent diabetes mellitus, multiple sclerosis, experimental allergic encephalomyelitis, Sjögren's syndrome, graft versus host disease, inflammatory bowel disease including Crohn's disease, ulcerative colitis, and scleroderma. Inflammatory diseases also includes autoimmune diseases such as myasthenia gravis, Guillain-Barré disease, primary biliary cirrhosis, hepatitis, hemolytic anemia, uveitis, Grave's disease, pernicious anemia, thrombocytopenia, Hashimoto's thyroiditis, oophoritis, orchitis, adrenal gland diseases, anti-phospholipid syndrome, Wegener's granulomatosis, Behcet's disease, polymyositis, dermatomyositis, multiple sclerosis, vitiligo, ankylosing spondylitis, Pemphigus vulgaris, psoriasis, and dermatitis herpetiformis.

The term “complement” refers to a complex group of proteins in body fluids that, working together with antibodies or other factors, play a role as mediators of immune, allergic, immunochemical and/or immunopathological reactions.

“Complement modulator” refers to any substance that has the ability to modulate the activity of complement. The complement modulator can include, but is not limited to, a complement inhibitor.

“Complement inhibitor” refers to any substance that has the ability to inhibit the activity of complement. The percentage of complement activity that is inhibited by the complement inhibitor can be less than 1%, less than 5%, less than 10%, less than 20%, less than 30%, less than 40%, less than 50%, less than 60%, less than 70%, less than 80%, less than 90%, or less than or equal to 100% inhibition of the complement.

“Infectious process” is defined as the process by which one organism is invaded by any type of foreign material or another organism. The results of an infection can include growth of the foreign organism, the production of toxins, and damage to the host organism.

“Liver toxicity” is defined as an abnormal accumulation of toxic substances in the liver. A number of criteria can be used to assess the clinical significance of toxicity data: (a) type/severity of injury, (b) reversibility, (c) mechanism of toxicity, (d) interspecies differences, (e) availability of sensitive biomarkers of toxicity, (e) safety margin (non toxic dose/pharmacologically active dose), and (f) therapeutic potential.

“Cancer therapy” is defined as any treatment or therapy useful in preventing, treating, or ameliorating the symptoms associated with cancer. Cancer therapy can include, but is not limited to, apoptosis induction, radiation therapy, and chemotherapy.

“Transplant” is defined as the transplantation of an organ or body part from one organism to another.

“Transplant rejection” is defined as an immune response triggered by the presence of foreign blood or tissue in the body of a subject. In one example of transplant rejection, antibodies are formed against foreign antigens on the transplanted material.

“Detecting inflammation” is defined as the process whereby inflammation is detected. Inflammation can be detected by a number of methods described herein, and can be in vivo, ex vivo, or in vitro.

By “isolated nucleic acid” is meant a nucleic acid, the structure of which is not identical to that of the naturally occurring nucleic acid or to that of any fragment of the naturally occurring genomic nucleic acid spanning more than three separate genes. The term therefore covers, for example, (a) a DNA which has the sequence of part of the naturally occurring genomic DNA molecules but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as cDNA, a genomic fragment, a fragment produced by polymerase chain reaction, or a restriction fragment; and (d) a recombinant nucleotide sequence that is part of a hybrid gene, i.e., a gene encoding a fusion protein.

By “label” is meant any detectable tag that can be attached directly (e.g., a fluorescent molecule integrated into a polypeptide or nucleic acid) or indirectly (e.g., by way of activation or binding to an expressed genetic reporter, including activatable substrates, peptides, receptor fusion proteins, primary antibody, or a secondary antibody with an integrated tag) to the molecule of interest. A “label” is any tag that can be visualized with imaging methods. The detectable tag can be a radio-opaque substance, radiolabel, a fluorescent label, a light emitting protein, a magnetic label, or microbubbles (air filled bubbles of uniform size that remain in the circulatory system and are detectable by ultrasonography, as described in Ellega et al. Circulation, 108:336-341, 2003, which is herein incorporated in its entirety). The detectable tag can be selected from the group consisting of gamma-emitters, beta-emitters, and alpha-emitters, positron-emitters, X-ray-emitters, ultrasound reflectors (microbubbles), and fluorescence-emitters suitable for localization. Suitable fluorescent compounds include fluorescein sodium, fluorescein isothiocyanate, phycoerythrin, Green Fluorescent Protein (GFP), Red Fluorescent Protein (RFP), Texas Red sulfonyl chloride (de Belder & Wik, Carbohydr. Res.44(2):251-57 (1975)), as well as compounds that are fluorescent in the near infrared such as Cy5.5, Cy7, and others. Also included are genetic reporters detectable following administration of radiotracers such as hSSTr2, thymidine kinase (from herpes virus, human mitochondria, or other) and NIS (iodide symporter). Light emitting proteins include various types of luciferase. Those skilled in the art will know, or will be able to ascertain with no more than routine experimentation, other fluorescent compounds that are suitable for labeling the molecule.

“Operably linked” is defined as the expression of a nucleic acid under the control of a given promoter sequence; i.e., the promoter controls the expression of a given nucleic acid. The given nucleic acid can be, but is not limited to, a reporter nucleic acid.

The term “promoter” is defined as a DNA regulatory region capable of binding RNA polymerase in a cell and initiating transcription of a downstream (3′ direction) coding sequence.

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and, more preferably, a human.

B. Methods of Using

Herein are disclosed methods of monitoring inflammation by imaging. The imaging can monitor in vivo, ex vivo, or in vitro systems. The genetic method involves using a “sensing” promoter (e.g., cox2L, cox2M, or others) to control one or more reporter genes such as luciferase, GFP, RFP, hSSTr2, TK, or other fluorescent, bioluminescent, or other reporters that can be imaged. This genetic construct is delivered to cells or tissues, and expression of the reporter is detected by in vivo imaging; the intensity of imaging signal being related to inflammation. Normal tissues without inflammation have low signal. The method also includes linking multiple reporter genes by IRES or other control elements, with control afforded by the inflammation inducible promoter element.

For certain approaches, delivery of the vector encoding the genetic construct is by intravenous route for delivery to liver/spleen, by aerosol/tracheal route for lung delivery, by intraperitoneal route for peritoneal delivery, by intramuscular route for muscle, and direct joint injection for synovial targeting, for example. Other approaches deliver the gene to cells before the cells are delivered to a subject, or to tissues prior to implantation. Still further approaches include transgenic animals with the promoter-reporter specifically targeted to particular cell types and tissues, as well as cell lines with the promoter-reporters described herein.

This technology can be applied in combination with other imaging or diagnostic technologies. For example, tumor mass can be assessed using tumor cells positive for CMV-luciferase. In addition, two luciferase enzymes can be imaged at the same time, for example, using CMV-luciferase (from firefly) and cox2L-luciferase (from Renilla). Furthermore, other reporters (e.g. GFP, RFP, hSSTr2, or other fluorescent reporters) are also useful with the methods described herein and can be used in any combination. For example, constructs with cox2L-hSSTr2 are able to detect inflammatory changes in liver using radiolabeled hSSRTr2-avid ligands or hSSTr2-avid ligands labeled with fluorophores (including near infrared fluorescent probes). Secreted embryonic alkaline phosphatase (SEAP) can also be controlled in the system. In this manner a blood-based reporter can be detected with a simple blood test to determine the activation status of the promoter, with subsequently identification of the location by imaging.

The present invention includes a method of detecting inflammation in a subject comprising administering to said subject a vector, said vector comprising a reporter nucleic acid operably linked to a promoter nucleic acid, wherein said reporter nucleic acid is expressed under conditions of inflammation; and detecting expression of said reporter nucleic acid by in vivo monitoring.

The present invention also contemplates a method of detecting inflammation in a transplant recipient comprising administering to cells of the transplant, prior to transplantation, a vector, said vector comprising a reporter nucleic acid and a promoter nucleic acid, wherein expression of said reporter nucleic acid is detectable under conditions of inflammation; performing the transplant; and detecting expression of said reporter nucleic acid by in vivo monitoring.

The present invention also includes a method of monitoring inflammation in a subject with an inflammatory or autoimmune disease, comprising administering to said subject a vector, said vector comprising a reporter nucleic acid operably linked to a promoter nucleic acid, wherein expression of said reporter nucleic acid is detectable under conditions of inflammation; and detecting expression of said reporter nucleic acid by in vivo monitoring.

The present invention also includes a method of treating a subject with an inflammatory disease comprising administering to said subject a vector, said vector comprising a reporter nucleic acid operably linked to a promoter nucleic acid, wherein said reporter nucleic acid is expressed under conditions of inflammation; detecting expression of said reporter nucleic acid by in vivo monitoring; and modifying treatment of the subject when expression of said reporter nucleic acid is detected.

Also contemplated by the present invention is a method of identifying a vector capable of detecting inflammation, comprising administering a vector to a cell culture, wherein the vector comprises a promoter nucleic acid and a reporter nucleic acid; inducing an inflammatory response in said cell culture; and monitoring expression of the reporter nucleic acid, expression indicating a vector capable of detecting inflammation.

The present invention also includes a method of monitoring inflammation in a subject with an inflammatory or autoimmune disease. The method comprises administering to the subject a vector, the vector comprising a reporter nucleic acid operably linked to a promoter nucleic acid, wherein expression of said reporter nucleic acid is detectable under conditions of inflammation; and detecting expression of said reporter nucleic acid by in vivo monitoring.

The present invention also relates to a method of identifying vectors that are capable of detecting inflammation. The method comprises administering a vector to a cell culture, wherein the vector comprises a promoter nucleic acid and a reporter nucleic acid; inducing an inflammatory response in the cell culture; and monitoring expression of the reporter nucleic acid, expression indicating a vector capable of detecting inflammation.

The following embodiments are applicable to any of the methods described above.

The vector can be any vector capable of delivering a nucleic acid to a subject. Optimally, the vector is a viral vector. For example, the viral vector can be a recombinant adenovirus vector, an adeno-associated viral vector, a lentiviral vector, a pseudotyped retroviral vector, a vaccinia vector, an alphavirus vector, as described above, or any other viral vector known in the art. Various vectors and their uses are described throughout.

A specific example of an adenoviral vector is adenovirus subtype 5. Adenovirus subtype 5 is a non-enveloped DNA virus. The structure of the vector is an icosahedral capsid (˜900 in diameter) that includes 12 vertices, from which extend trimeric fiber proteins that end with trimeric knobs. The Ad vector can be replication incompetent, due to deletions in the viral genome (E1 and E3) to allow insertion of the reporter cassettes. The Ad vector can also be replication competent so that the vector can conditionally replicate within the subject.

The genetic code of the Ad can be modified to change the natural tropism of the vector. Infection of mammalian cells is mediated (in part) by the knob structure of the Ad virion that recognizes and binds to the coxsackie adenoviral receptor (CAR) on the cell surface (Bergelson et al., Science 275: 1320-1323, 1997) and facilitates interaction with tissue integrins as part of internalization. The knob structures can be genetically modified to ablate binding to CAR (Einfeld et al., J. Virol. 75: 11284-91, 2001). The structure in Ad responsible for binding to tissue integrins can also be ablated. Vectors that lack CAR and integrin binding are termed “double ablated” vectors. The KKTK peptide motif structure (SEQ ID NO: 1) in the Ad5 fiber shaft has been identified as mediating binding to heparin sulfate proteoglycans expressed in the liver (Smith et al., Mol. Ther. 5:S149, 2000.) This sequence can be altered genetically to change the natural tropism of the Ad to reduce liver accumulation. Genetic manipulation of Ad can also add new targeting motifs. For example, new sequences for targeting can be included in the loop structure of the knob. (Krasnykh et al., J. Virol. 70:6839-46, 1996; Krasnykh et al., J. Virol. 72:1844-52, 1998; Krasnykh et al., Cancer Res., 60:6784-7, 2000; Hemminki et al., Proc. Of ASCO 21a:82, 2000.) One example of the loop insertion is the tripeptide “RGD” sequence (SEQ ID NO: 2). “RGD” Ad vectors show increased binding to integrins that are expressed at a higher level on cancer cells and tumor vasculature, with which inflammation is associated. This binding is accompanied by increased infection of the cancer cells, even in the presence of neutralizing antibody against the Ad vector (Biermann et al., Hum. Gene Ther. 12:1757-69, 2001, Hemminki et al., J. Nat'l. Cancer Inst. 94: 74 1-9, 2002). Another example of genetic manipulation that led to addition of a targeting motif is fiber-fibritin (EF) chimeras (Krasnykh et al., J. Virol. 75:4176-83, 2001). In this regard, FF-containing Ad vectors are unique in that they do not contain either the fiber knob or the KKTK (SEQ ID NO: 1) tetrapeptide in the shaft and therefore allow for bypassing the natural mechanism of the vector's sequestration in vivo. Therefore, the present invention also contemplates FF-containing Ad vectors, with tumor-targeting motifs and strategies to reduce the immune response to the vector.

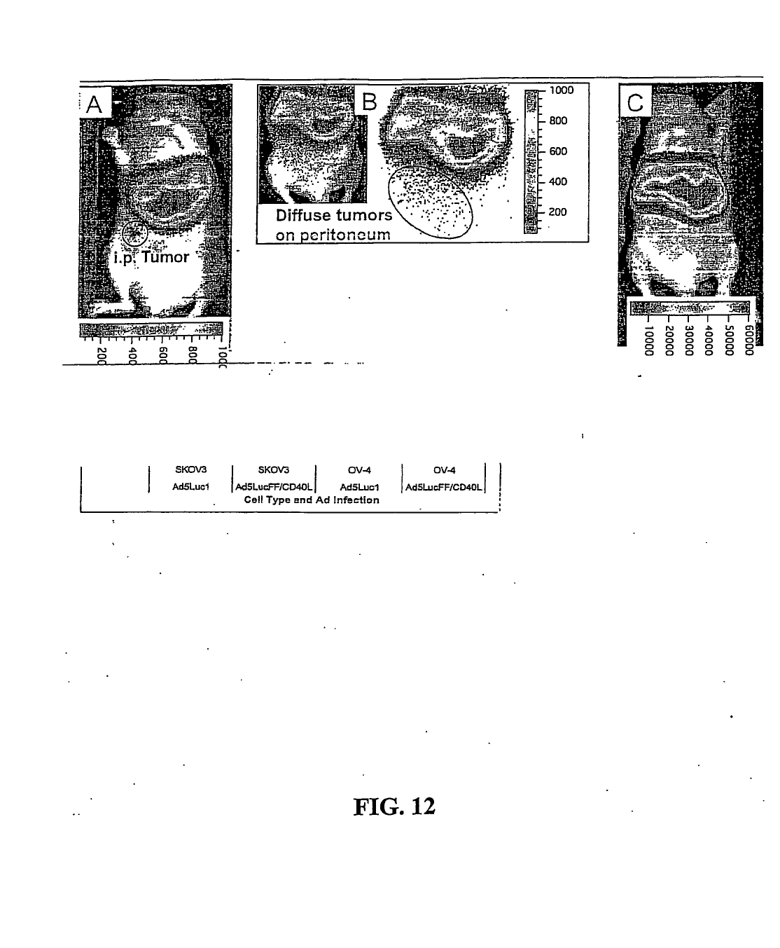

Certain areas within a subject can be targeted by directing the vector to the appropriate receptor. For example, the targeting of tumor endothelium enhances delivery of reporter and therapeutic genes to tumors. Receptors with high expression on the tumor endothelium can be targeted. This allows for access of the Ad vector to receptors expressed on tumor endothelium, an additional mechanism for infectivity besides leaky or compromised tumor vasculature. i.v. Targeting of CD40-positive ovarian xenografts, using an Ad with a FF chimera incorporating human CD40L (Ad5LucFF/CD40L) is disclosed in Example 7. CD40 is strongly expressed on many carcinomas (e.g., breast, ovarian, and lung) and melanomas (Tong et al., Cancer Gene Ther. 10:1-13, 2003; Thomas et al., Int. J. Cancer 68:795-801, 1996; Kluth et al., Cancer Res. 9:619-24, 2003) and shows high levels of expression on the tumor vasculature (Kluth et al., Cancer Res. 57:891-9, 1997; Tong et al., Clin. Cancer Res. 7:691-703, 2001). CD40-based treatment strategies use cross linking and immune activation strategies. Expression of CD40 on normal human endothelium was low or absent (Pammer et al., Am. J. Pathol. 148:1387-96, 1996; Pammer et al., Histopathology, 29:517-24, 1996; Maisch et al., J. Virol. 76:12803-12, 2002), further supported by lack of systemic toxicity associated with CD40L therapy in a phase I trial (Vonderheide et al., J. Clin. Oncol. 19:3280-7, 2001). i.v. Ad targeting (FIG. 12) is validated with the FF replacement strategy using CD40L. Preferably, the CD40L is of human origin and does not bind to mouse CD40 that is expressed on the tumor endothelium in mice.

One example of Ad-mediated targeting of inflammation associated with tumor endothelium is E-selectin. E-selectin activation can be imaged in an inflammatory model using Tc-99m-labeled peptides with high affinity for E-selection. The peptide sequences show high-affinity binding to mouse, rat, and human E-selectin, thereby providing an ideal situation for testing Ad targeting constructs (for example, FF chimeras with incorporated E-selectin targeting peptides). E-selectin is not expressed on normal endothelium, rather only in inflammatory process such as rheumatoid arritis or in the tumor endothelium Langley et al., Am. J. Physiol. 277:H1156-66, 1999; Nguyen et al., Am. J. Pathol. 150:1307-14, 1997; Staal-van den Brekel et al., Vrichows Arch. 428:21-7, 1996), and is an important receptor for angiogenesis (Aoki et al., Tumour Biol. 22:23946, 2001; Kraling et al Am. J. Pathol. 148:1181-91, 1996) and metastasis (Uotani et al., J. Surg. Res. 96:197-203, 2001; Kobayashi et al., Cancer Res. 60:3978-84, 2000; Matsumoto et al., Br. J. Cancer 86:161-7, 2002).

The somatostatin receptor (subtypes 2 and 5) can also be a target. Many tumors are positive for these receptors, and tumor vasculature also showed high expression (Cascini et al., Minerva Endocrinol. 26:129-33, 2001; Koh et al., Clin. Nucl. Med. 26:870-1, 2001; Cuntz et al., Ann. Surg. Oncol. 6:367-72, 1999; Watson et al., Br. J. Cancer 85:266-72, 2001). Rat adenocarcinoma mammary tumors induced with the carcinogen N-nitroso-N-methylurea (MNU) have high expression of the somatostatin receptors, as indicated by retention of a Tc-99m-labeled, SSTR-avid peptide (P2045). This is the peptide used for imaging hSSTr2 expression. The same peptide binds with high affinity to mouse, rat, and human SST receptors (subtypes 2 and 5).

The promoter can be any promoter which is capable of directing expression in the presence of inflammation. Examples of suitable promoters include, but are not limited to cyclooxygenase promoters. Cyclooxygenase is the rate-limiting step in the conversion of arachidonic acid to prostaglandins. There are two known genes of cyclooxygenase, Cox1 and Cox2. Cox1 is constitutively expressed at low levels in many cell types. Specifically, Cox1 is known to be essential for maintaining the integrity of the gastrointestinal epithelium. Cox2 expression is stimulated by growth factors, cytolines, and endotoxins. The cyclooxygenase 2 isoform (Cox2) is not expressed in most tissues (e.g., liver) under physiological conditions but is highly upregulated in inflammatory processes and cancer, for example. Up-regulation of Cox2 is responsible for the increased formation of prostaglandins associated with inflammation.

Examples of Cox2 promoters include, but are not limited to, cox2L promoters and cox2M promoters. The cox2L promoter element is not active in normal liver in the absence of inflammation. The cox2L promoter refers to the entire 5′ regulatory region that controls expression of the cyclooxygenase 2 enzyme as previously reported. (Inoue H, Yokoyama C, and Tanabe T, Structure and expression of an inducible prostaglandin endoperoxide synthase gene. Tanpakushitsu Kakusan Koso 40:399-408, 1995.; Inoue H, Yokoyama C, Hara S, Tone Y, and Tanabe T, Transcriptional regulation of human prostaglandin-endoperoxide synthase-2 gene by lipopolysaccharide and phorbol ester in vascular endothelial cells. Involvement of both nuclear factor for interleulin-6 expression site and cAMP response element. J Biol Chem 270:24965-71, 1995; Inoue H, Kosaka T, Miyata A, Hara S, Yokoyama C, Nanayama T, and Tanabe T, Structure and expression of the human prostaglandin endoperoxide synthase 2 gene. Adv Prostaglandin Thromboxane Leukot Res 23:109-11, 1995.)

Another example of a promoter that can be used with the above methods is the constitutive cytomegalovirus (CMV) promoter. An additional tissue specific promoter is flt-1, a promoter that is active in endothelial cells.

The vector can also comprise a reporter nucleic acid. The reporter nucleic acid can be any nucleic acid that encodes a molecule that allows for detection. It is understood that the reporter nucleic acid can be linked to the promoter nucleic acid. For example, the reporter nucleic acid can encode any chemiluminescent or bioluminescent molecule, but they could also be phosphorescent or radioactive, for example. Those of skill in the art will recognize that there are various reporter molecules and will know how to integrate them for use with the present compositions and methods. Specifically, the reporter nucleic acid can encode a fluorescent protein. Examples of such reporters include, but are not limited to green fluorescent protein (GFP), red fluorescent protein (RFP), human type 2 somatostatin receptor (hSSTr2), thymidine kinase (TK), cytosine deaminase (CD) and luciferase.

In another embodiment, expression of the reporter nucleic acid is detected by a labeled ligand for a polypeptide encoded by the reporter nucleic acid. Examples of labeled ligand include, but are not limited to, labeled somatostatin or unlabeled somatostatin that is bound by a labeled somatostatin, labeled somatostatin analogues, labeled FIAU, FAU, or related ligands specific for thymidine kinase, labeled iodide specific for the iodide symporter reporter, and labeled whole antibody or antibody fragments targeting CEA.

Another embodiment of the invention comprises a complement modulator. Inhibition of complement can be used to reduce redirection of the vector, thereby allowing its concentration in a desired location. Inhibition of complement can also be used as a method of treatment to reduce inflammation. Specifically, the complement modulator can inhibit complement activation. Complement is a complex system containing more than 30 various glycoproteins present in serum in the form of components, factors, or other regulators and/or on the surface of different cells in the form of receptors. These are present in the blood serum in an inactive state and are activated by immune complexes (the classical pathway), by carbohydrates (the lectin pathway), or by other substances, mainly of bacterial origin (the alternative pathway).

The components of the classical pathway are numbered 1 to 9 and prefixed by the letter C, e.g. C1, C2 . . . C9. C1 is composed of three subcomponents C1q, C1r, and C1s. The early components of the alternative pathway are known as factors, and each molecule is named by a letter, for example factor B, D, P. The lectin pathway is the same as the classical pathway, only C1q is omitted. All these pathways use in the later stages of activation the same terminal components C5-C9 that form membrane attack complex (MAC). C3 also participates in all pathways.

Activation of each of the components results from a proteolytic cleavage event in a cascade mechanism which fragments the native molecule into two fragments. The fragment which participates further in the complement cascade is designated the ‘b’ fragment (e.g. C3b) and is usually larger than the ‘a’ fragment (e.g. C5a) which possesses other biological activities.

Complement activation is a complex and redundant series of enzymatic reactions that converts pre-existing protein substrates into biologically active end products. For example, in a process called opsonization, the deposition of C3 fragments onto pathogens promotes the removal of the pathogens by the reticuloendothelial system. In gene therapy applications, redirection of the vector in this manner can lead to toxicity. Toxicity and accumulation in organs like the liver can occur even upon localized administration of a vector. Thus, even intramuscular or subcutaneous administration can result in leakage into the vascular system, resulting in toxicity and liver accumulation. The vectors disclosed herein can decrease these effects for gene therapy vectors.

Equally important, when vector is removed by liver, less vector remains available for transfecting the desired target cell population. Consistent with this view, Wilson et al. reported greater reporter expression in mouse hepatocytes following systemic Ad administration with high vector doses that saturated Kupffer cells (Tao (2001) Mol. Ther. 3:28-35). This was true even with doses that included a different Ad without the reporter construct.

The liver is the predominant site of reporter gene expression following intravenous injection of wild-type Ad5 vectors (Einfeld (2001) J Virol 75:11284-11291). As mentioned above, there is also an accumulation of reporter gene expression in the liver following subcutaneous injection of unmodified vectors, due to release from the local injection to the systemic circulation. Coxsackie and adenovirus receptor (CAR), integrins, and heparin sulfate proteoglycans have all been shown to be important for liver transfection (Kirby (2000) J Virol 74:2804-2813; Kirby (1999) J Virol 73: 9508-9514; Santis (1999) J Gen Virol 80 (Pt 6):1519-1527; Nakamura (2003) J Virol 77: 2512-2521.) Ad vectors with CAR binding site mutations and ablation of integrin-binding showed less luciferase expression in liver following systemic administration. Similarly, ablation of CAR-binding via short fiber replacements also lead to reduced liver tropism. Furthermore, blood coagulation factor IX is also involved in liver transduction (Shayakhmetov (2003) Mol. Ther. 7:S165). The humoral immune response also influences liver transgene expression, especially when the host is repeatedly exposed to the vector, because neutralizing antibody can diminish liver transfection.

Complement plays an important role in the transduction of mouse liver by Ad. To directly address the role of complement in liver transduction, experiments were performed using wild type mice versus mutant mice unable to make complement component 3 (C3) (Circolo (1999) Immunopharmacology, 42:135-49). By repeated bioluminescence imaging of living mice, liver luciferase expression was assessed following intravenous delivery of the Ad vector. At low Ad doses, C3 deficient mice (C3^{-/-}) showed up to 99-fold less luciferase expression in the liver compared to wild type controls, indicating a facilitator role for the complement pathway in liver transduction. (Example 1). C3^{-/-} mice were used with the C57BL/6 background, together with littermate controls matched for sex and age.

Innate and systemic immunity can be considered in the design of the vector. The goal to achieve vector targeting will not be realized if the vector is opsonized by the innate immune system. Therefore, complement plays a role in the removal of vectors following systemic administration, especially with respect to complement-mediated transduction of the liver. The role of complement is important in systemic viral targeting of cancer and inflammation (Ikeda et al., J. Virol. 74:4765-75, 2000, Cichon et al., Gene Ther. 8:1794-800, 2001) and in regard to the immune response to virus (Suresh et al., J. Immunol. 170:788-94, 2003). As shown in Example 1, C3 knockout mice showed significantly lower levels of reporter gene expression, and required longer times to detect the expression compared to non-C3 knockout mice.

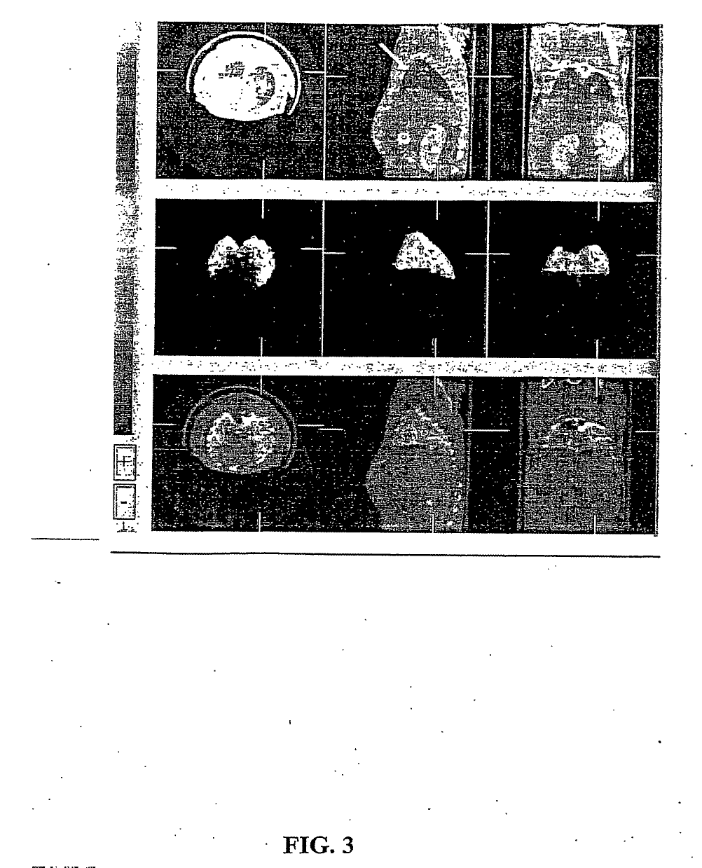

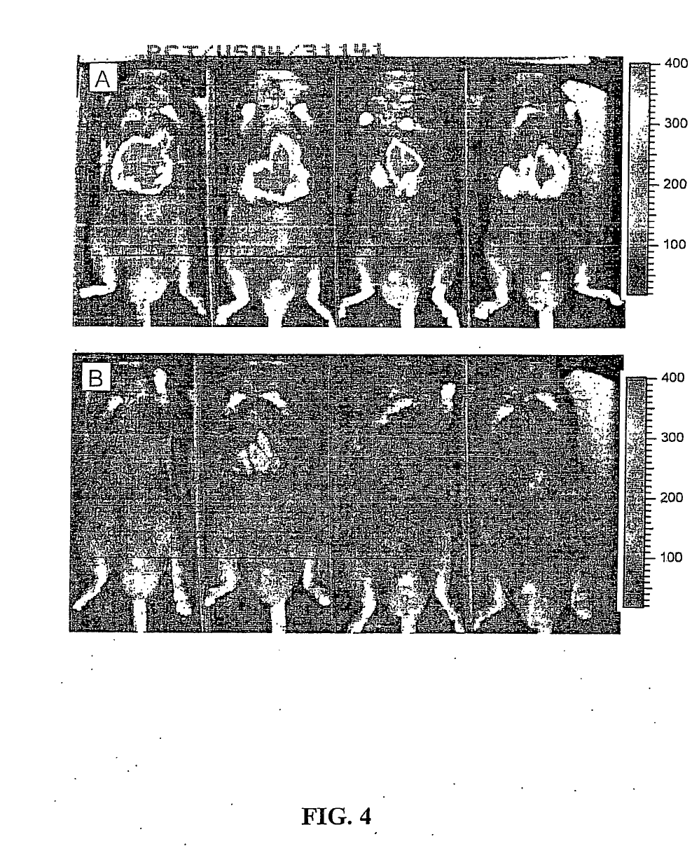

Two general strategies exist for the reduction of immune activation that accompany viral vector delivery. In the first strategy, the vector is modified to include the genetic code for amino acid sequences that are displayed on the surface of the vector and thereby bind factors that are either negative regulators of human complement or result in functional inhibition of the human complement cascade. An example of the functional inhibition is demonstrated by the ed1 region of the Sh-TOR protein of the Schistosoma parasite; the ed1 region is a N-terminal peptide of 26 amino acids that binds human complement component 2 (Inal et al, FEBS Letters 470: 131-134, 2000, FIG. 4, herein incorporated by reference in its entirety for the sequence and variations thereof). The last 11 amino acids (SEQ ID NO: 10) of the Sh-TOR is similar to that used in SEQ ID NO: 9, but it was used in duplicate and spacer amino acids were added. The binding inhibits the classical complement-activation pathway by interfering with the formation of the C3-convertase complex and allows survival of the organism in the blood (Oh et al., Immunology 110:73-79, 2003, FIG. 3, herein incorporated by reference in its entirety for its teaching regarding rH17d′ (SEQ ID NO: 11).

Herein disclosed is an amino acid sequence comprising at least two repeats of ED1, optionally with linker peptides before, after, or between the repeats and a His-tag before, after, or between the repeats. For example, disclosed herein is SEQ ID NO: 9 (LGS-HEVKIKHFSPY-HEVKIKHFSPY-GS-HHHHHH-LGS) and nucleic acids that encode it, wherein HEVKIKHFSPY is ED1, wherein LGS and GS are linker peptides, and wherein the His-tag is a 6 His-tag. The polypeptide regulates complement, and can be inserted into adenovirus hypervariable regions, AAV surface proteins, or generally in surface proteins of a wide range of gene therapy vectors. The polypeptide is encoded, for example, by the nucleic acid sequence designated as SEQ ID NO: 8. In particular, HVR2 and HVR5 can be used as sites of insertion (Example 16). With respect to adenovirus, other hypervariable regions (besides 2 and 5) are likely to be equally suited for these insertions, and insertions in multiple HVR regions are desirable. Further, other Ad coat proteins, including pIX, are also applicable for this genetic insertion and resulting down-modulation of complement. Various linker sequences are applicable, including GG, GGG, GGGG, or longer G inserts, SS, SSS, SSS, or longer S inserts, GGS, GGSS, various G and S combinations, as well as other amino acids with minimal sidechains that do not disrupt the 3-D structure of the ED1 insert. A trimeric ED1 insert can also be used. Rux et al. (J. Virol. 77:9553-9566, 2003, and Mol. Ther. 1:18-30, 2000, herein incorporated by reference in their entirety for their teaching regarding HVR) show the HVR regions and sequence variation of these regions.

Examples of negative regulators of complement are complement regulator Factor H or C4b, human complement regulators that bind to protein molecules (for example, M-proteins, Bac or Beta, or PspC) that are located on the surface of group A streptococcus, group B streptococcus, and pneumococcus. Binding of the human complement regulators by the pathogens are a primary mechanism to evade the human immune response (for review, see Jarva et al, Molecular Immunology 40:95-107, 2003). An example of a surface site for incorporation of the amino acid sequences in the Ad vector includes PIX, a site demonstrated to allow for genetic addition of proteins. A linker site (poly GGGGS) (SEQ ID NO: 3) between the FF chimera and retargeting ligands is a second site that can be utilized. A third site is the hexon structural protein, especially the hypervariable regions, as described in the previous section. In all of these examples, negative regulators of complement activation will bind the surfaces of the Ad vector (or other gene therapy vectors), and thereby reduce complement activation. This strategy is used by certain microorganisms to bypass innate immunity.

The second strategy is to encode the negative regulators of complement directly within the genome of the vector so the negative regulators become displayed on the viral surface. In this manner, no binding of a blood factor is necessary, as the factor is displayed by the vector when the vector is assembled. An example of a negative regulatory protein displayed in this manner is the Crry protein, a complement inhibitor protein that has worked for this purpose in several model systems (Caragine et al., Cancer Res. 62:1110-5, 2002, Caragine et al., Blood 100:3304-10, 2002, Quigg et al., J. Immunol. 155:1481-8, 1995). Expression of Crry on human MCF7 cancer cells inhibited complement activation (C3) and increased the tumorigenicity of the MCF-7 cells in a rat breast cancer model (Caragine et al., Cancer Res. 62:1110-5, 2002). A second example is the carboxy-terminal domain of complement factor H, the structural element responsible for inhibition of the alternate pathway of complement (Hellwage et al, J Immunol 169: 6935-6944, 2002).

The significance of complement to humoral immune response is found in Ochsenbein et al (J Exp Med 190:1165-74, 1999). It was shown that activation of complement was necessary for an efficient immune response to vesicular stomatitis virus, poliomyelitits virus, and recombinant vaccinia.

Inflammation can be associated with a number of different diseases and disorders. Examples of inflammation include, but are not limited to, inflammation associated with hepatitis, inflammation associated with the lungs, and inflammation associated with an infectious process. Inflammation can also be associated with liver toxicity, which can be associated in turn with cancer therapy, such as apoptosis induction or chemotherapy, or a combination of the two, for example.

When the inflammation is associated with an infectious process, the infectious process can be associated with a viral infection. Examples of viral infections include, but are not limited to, Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B. Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, and Human Immunodeficiency virus type-2.

The infectious process can also be associated with a bacterial infection. Examples of bacterial infections include, but are not limited to, M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

The infectious process can also be associated with a parasitic infection. Examples of parasitic infections include, but are not limited to, Toxoplasma gondii, Plasmodium species such as Plasmodium falciparum, Plasmodium vivax, Plasmodium malariae, and other Plasmodium species, Trypanosoma brucei, Trypanosoma cruzi, Leishmania species such as Leishmania major, Schistosoma such as Schistosoma mansoni and other Shistosoma species, and Entamoeba histolytica.

The infectious process can also be associated with a fungal infection. Examples of fungal infections include, but are not limited to, Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternata.

The inflammation can be associated with an inflammatory disease. Examples of inflammatory disease include, but are not limited to, asthma, systemic lupus erythematosus, rheumatoid arthritis, reactive arthritis, spondyarthritis, systemic vasculitis, insulin dependent diabetes mellitus, multiple sclerosis, experimental allergic encephalomyelitis, Sjögren's syndrome, graft versus host disease, inflammatory bowel disease including Crohn's disease, ulcerative colitis, and scleroderma. Inflammatory diseases also includes autoimmune diseases such as myasthenia gravis, Guillain-Barr\acute{e} disease, primary biliary cirrhosis, hepatitis, hemolytic anemia, uveitis, Grave's disease, pernicious anemia, thrombocytopenia, Hashimoto's thyroiditis, oophoritis, orchitis, adrenal gland diseases, anti-phospholipid syndrome, Wegener's granulomatosis, Behcet's disease, polymyositis, dermatomyositis, multiple sclerosis, vitiligo, ankylosing spondylitis, Pemphigus vulgaris, psoriasis, and dermatitis herpetiformis.

The inflammation can be associated with cancer. Examples of types of cancer include, but are not limited to, lymphoma (Hodgkins and non-Hodgkins) B-cell lymphoma, T-cell lymphoma, leukemia such as myeloid leukemia and other types of leukemia, mycosis fungoide, carcinoma, adenocarcinoma, sarcoma, glioma, blastoma, neuroblastoma, plasmacytoma, histiocytoma, melanoma, adenoma, hypoxic tumour, myeloma, AIDS-related lymphoma or AIDS-related sarcoma, metastatic cancer, bladder cancer, brain cancer, nervous system cancer, squamous cell carcinoma of the head and neck, neuroblastoma, glioblastoma, ovarian cancer, skin cancer, liver cancer, squamous cell carcinomas of the mouth, throat, larynx, and lung, colon cancer, cervical cancer, breast cancer, cervical carcinoma, epithelial cancer, renal cancer, genitourinary cancer, pulmonary cancer, esophageal carcinoma, head and neck carcinoma, hematopoietic cancer, testicular cancer, colo-rectal cancer, prostatic cancer, and pancreatic cancer.

Activated cells can also be identified at the site of inflammation. “Activated cells” are defined as cells that participate in the inflammatory response. Examples of such cells include, but are not limited to, T-cells and B-cells, macrophages, NK cells, mast cells, eosinophils, neutrophils, Kupffer cells, antigen presenting cells, as well as vascular endothelial cells.

Inflammation can also be associated with transplant rejection in a transplant recipient. As disclosed above, “transplant rejection” is defined as an immune response triggered by the presence of foreign blood or tissue in the body of a subject. In one example of transplant rejection, antibodies are formed against foreign antigens on the transplanted material. The tratransplantation can be, for example, organ transplantation, such as liver, kidney, skin, eyes, heart, or any other transplantable organ of the body or part thereof.

Transplantation immunology refers to an extensive sequence of events that occurs after an allograft or a xenograft is removed from a donor and then transplanted into a recipient. Tissue is damaged at both the graft and the transplantation sites. An inflammatory reaction follows immediately, as does activation of biochemical cascades. A series of specific and nonspecific cellular responses ensues as antigens are recognized. Antigen-independent causes of tissue damage (i.e., ischemia, hypothermia, reperfusion injury) are the result of mechanical trauma as well as disruption of the blood supply as the graft is harvested. In contrast, antigen-dependent causes of tissue damage involve immune-mediated damage.

Macrophages release cytokines (e.g., tumor necrosis factor, interleukin-1), which heighten the intensity of inflammation by stimulating inflammatory endothelial responses; these endothelial changes help recruit large numbers of T cells to the transplantation site.

Damaged tissues release pro-inflammatory mediators (e.g., Hageman factor (factor XII) that trigger several biochemical cascades. The clotting cascade induces fibrin and several related fibrinopeptides, which promote local vascular permeability and attract neutrophils and macrophages. The kinin cascade principally produces bradykinin, which promotes vasodilation, smooth muscle contraction, and increased vascular permeability.

Rejection is the consequence of the recipient's alloimmune response to the nonself antigens expressed by donor tissues. In hyperacute rejection, transplant subjects are serologically presensitized to alloantigens (i.e., graft antigens are recognized as nonself). Histologically, numerous polymorphonuclear leukocytes (PMNs) exist within the graft vasculature and are associated with widespread microthrombin formation and platelet accumulation. Little or no leukocyte infiltration occurs. Hyperacute rejection manifests within minutes to hours of graft implantation. Hyperacute rejection has become relatively rare since the introduction of routine pretransplantation screening of graft recipients for antidonor antibodies.

In acute rejection, graft antigens are recognized by T cells; the resulting cytokine release eventually leads to tissue distortion, vascular insufficiency, and cell destruction. Histologically, leukocytes are present, dominated by equivalent numbers of macrophages and T cells within the interstitium. These processes can occur within 24 hours of transplantation and occur over a period of days to weeks.

In chronic rejection, pathologic tissue remodeling results from peritransplant and posttransplant trauma. Cytokines and tissue growth factor induce smooth muscle cells to proliferate, to migrate, and to produce new matrix material. Interstitial fibroblasts are also induced to produce collagen. Histologically, progressive neointimal formation occurs within large and medium arteries and, to a lesser extent, within veins of the graft. Leukocyte infiltration usually is mild or even absent. All these result in reduced blood flow, with subsequent regional tissue ischemia, fibrosis, and cell death. (Prescilla et al. http://www.emedicine.com, Immunology of Transplant Rejection, updated Jun. 20, 2003).

Transplant rejection may occur within 1-10 minutes of transplantation, or within 10 minutes to 1 hour of transplantation, or within 1 hour to 10 hours of transplantation, or within 10 hours to 24 hours of transplantation, within 24 hours to 48 hours of transplantation, within 48 hours to 1 month of transplantation, within 1 month to 1 year of transplantation, within 1 year to 5 years of transplantation, or even longer after transplantation.

As disclosed above, inflammation can be monitored using in vivo monitoring. Sensitive detection devices can be employed to visualize and quantify light or other forms of emission by detecting photons or other signals that are transmitted through mammalian tissue from internal sources. Weak visible light sources can be imaged using charged coupled device (CCD) cameras, for example, and can include microchannel plate intensifiers, Peltier or liquid nitrogen cooling of the detector, and a combination where the intensifier, and not the CCD detector, is cooled. The goal of these technologies is to enhance signal to noise ratios by either reducing background (cooled) or increasing signal (intensified).

In vivo monitoring can be carried out using, for example, bioluminescence imaging, planar gamma camera imaging, SPECT imaging, light-based imaging, magnetic resonance imaging and spectroscopy, fluorescence imaging (especially in the near infrared), diffuse optical tomography, ultrasonography (including untargeted microbubble contrast, and targeted microbubble contrast), PET imaging, fluorescence correlation spectroscopy, in vivo two-photon microscopy, optical coherence tomography, speckle microscopy, small molecule reporters, nanocrystal labeling and second harmonic imaging Using the aforementioned imaging technologies, reporter genes under control of various inflammation specific promoters are detected following specific induction. Specifically, the type 2 somatostatin receptor (hSSTr2) is detected by gamma camera and SPECT imaging, fluorescence imaging, and PET. Microbubble contrast specifically targeted to hSSTr2 by various means allows ultrasonography to be applied for detection of the hSSTr2 as well.

Imaging can be carried out using single photon three-dimensional (3-D) emission computed tomography (SPECT). SPECT provides a qualitative and quantitative look at the volume distribution of biologically significant radiotracers after injection into the human body. Three-dimensional SPECT, a process involving rotation of up to three photon-sensitive cameras (Gamma cameras) around a subject, results in a 3-D image of the distribution of an injected radiotracer which is usually targeted for a particular organ, for example the liver. The 3-D image thus obtained is the result of reconstructing a series of 2-D projection sets, then “stacking” these one on top of the next to create the third dimension.

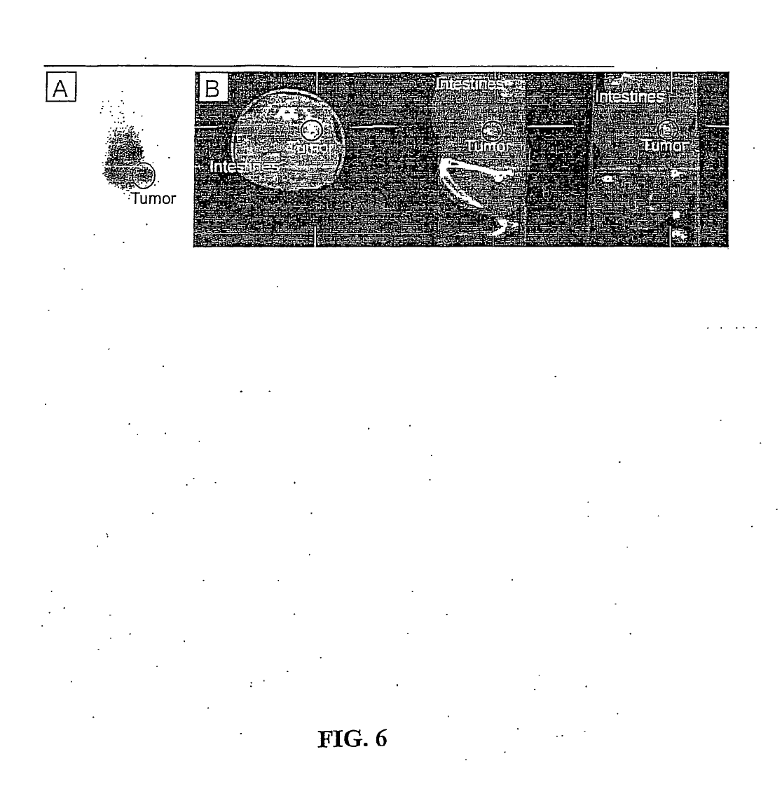

There is a capability to image at ˜0.8 mm 3-dimensional resolution for the SPECT component. The emission SPECT images are accurately fused with the anatomic CT image, as shown in the example (FIG. 6). In this example the mouse was i.v. injected with Tc-99m-labeled macroaggregated albumin (MAA, 300 microcuries) microspheres that are trapped in the capillaries of the lung. The SPECT imaging session required ˜30 minutes to acquire 64 views. As shown, the fusion images accurately reveal the expected distribution of the Tc-99m-MAA throughout the entire lung. The SPECT/CT fusion is important for accurately determining the location of Tc-99m-labeled radiotracers at 1-mm resolution in the mouse, for example, to determine the precise location of Tc-99m-labeled Ad vectors, or hSSTr2 transgene expression by imaging specific retention of the Tc-99m-labeled hSSTr2-avid peptide.

Imaging can also be carried out using Positron Emission Topography (PET). PET is a technique in which radioisotopes that emit positrons are used in conjunction with a promoter in a subject. The collision of a positron and an electron in the subject results in the emission of gamma rays, which can be detected and used to note the location of various processes, including inflammation.

These technologies can be applied in combination with other imaging technologies. For example, tumor mass monitoring can be accomplished using tumor cells positive for CMV-luciferase. In addition, two luciferase enzymes can be imaged at the same time, for example, using CMV-luciferase (from firefly) and cox2L-luciferase (from Renilla). Other reporters and promoters can be used in conjunction with these examples, some examples of which are disclosed above.

Also contemplated in the present invention is that the vector can further comprise a nucleic acid that encodes a detectable secreted protein. An example of a detectable secreted protein includes, but is not limited to, secreted embryonic alkaline phosphatase (SEAP). Expression of the reporter nucleic acid can be assessed by detecting the secreted protein. Using a secreted protein allows for a blood test to determine the activation status of the promoter, with subsequent identification of the location by imaging. Using a detectable secreted protein can allow for blood-based screening in conjunction with the use of another reporter useful in in vivo monitoring. An example of using a dual reporter system is described by Chaudhuri et al. (Tech in Cancer Res and Treat, 2(2):1-9, 2003).

C. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that, while specific reference of each various individual and collective combinations and permutation of these compounds may not be explicitly disclosed, each is specifically contemplated and described herein. For example, if a particular vector is disclosed and discussed and a number of modifications that can be made to a number of places within the vector can be made, including the portion encoding the reporter or the promoter, as well as the portion encoding the secreted protein, are discussed, specifically contemplated is each and every combination and permutation of the promoter, the reporter and/or the secreted protein, and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Disclosed are isolated nucleotide sequences representing the vectors of the invention. For example, the invention provides a vector comprising a reporter nucleic acid operably linked to a promoter nucleic acid. Also disclosed are recombinant host cells comprising the vector comprising a reporter and a promoter as disclosed herein. Also provided are expression vectors, wherein the expression vector is operable in prokaryotic or eukaryotic cells. Further provided are nucleic acid sequences that selectively hybridize under stringent conditions with the nucleic acids that encode the vectors of the invention.

In one embodiment, the invention provides a composition comprising the vector and an auxiliary protein that is required to enter the appropriate environment.

1. Sequence Similarities

Disclosed herein are vectors, promoters, reporters, and secreted proteins with nucleic acid or amino acid sequences that are similar to the sequences disclosed herein. It is understood that, as discussed herein, the use of the terms “homology” and “identity” are used interchangeably with “similarity” with regard to amino acid or nucleic acid sequences. Homology is further used to refer to similarities in secondary and tertiary structures. In general, it is understood that one way to define any known variants and derivatives or those that might arise, of the disclosed genes and proteins herein, is through defining the variants and derivatives in terms of similarity to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent similarity to the stated sequence or the native sequence. For example, SEQ ID NO: 5 sets forth a particular nucleic acid sequence for the vector Ad5LucI. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent similarity to the stated sequence. Those of skill in the art readily understand how to determine the similarity of two nucleic acids, such as genes. For example, the similarity can be calculated after aligning the two sequences so that the similarity is at its highest level.

Another way of calculating similarity can be performed by published algorithms. Optimal alignment of sequences for comparison may be conducted by the algorithm of Smith and Waterman Adv. Appl. Math. 2: 482 (1981), by the alignment algorithm of Needleman and Wunsch, J. Mol Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of similarity can be obtained for nucleic acids by for example the algorithms disclosed in Zuker, M. Science 244:48-52, 1989, Jaeger, Proc. Natl. Acad. Sci. USA 86:7706-7710, 1989, Jaeger, Methods Enzymol. 183:281-306, 1989, which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent similarity to another sequence refers to sequences that have the recited homology as calculated by any one or more of the calculation methods described above. For example, a first sequence has 80 percent similarity, as defined herein, to a second sequence if the first sequence is calculated to have 80 percent similarity to the second sequence using the Zuker calculation method even if the first sequence does not have 80 percent similarity to the second sequence as calculated by any of the other calculation methods. As another example, a first sequence has 80 percent similarity, as defined herein, to a second sequence if the first sequence is calculated to have 80 percent similarity to the second sequence using both the Zuker calculation method and the Pearson and Lipman calculation method even if the first sequence does not have 80 percent similarity to the second sequence as calculated by the Smith and Waterman calculation method, the Needleman and Wunsch calculation method, the Jaeger calculation methods, or any of the other calculation methods. As yet another example, a first sequence has 80 percent similarity, as defined herein, to a second sequence if the first sequence is calculated to have 80 percent similarity to the second sequence using each of calculation methods (although, in practice, the different calculation methods will often result in different calculated similarity percentages).

Other structural similarities, aside from sequence similarity are also disclosed. For example, homology, as noted by similar secondary and tertiary structure can be analyzed, as taught herein. Homologous proteins may have minimal sequence similarity but have a homologous catalytic domain. Thus, vectors as used herein may be structurally similar based on the structure of the reporter or promoter but have lower than 70% sequence similarity.

2. Hybridization/selective Hybridization

The term “hybridization” typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 5-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook, Molecular Cloning: A Laboratory Manual, 2nd Ed., Cold Spring Harbor Laboratory, Cold Spring Harbor, N.Y., 1989; Kunkel, Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their kd, or where only one of the nucleic acid molecules is 10 fold or 100 fold or 1000 fold or where one or both nucleic acid molecules are above their kd.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with similarity, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

3. Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, secreted proteins for detection, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with, for example, the promoter, such as a Cox2L or a Cox2M promoter, a reporter, or any other disclosed molecule. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAse H mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (kD) less than 10−6. It is more preferred that antisense molecules bind with a kD less than 10−8. It is also more preferred that the antisense molecules bind the target molecule with a kD less than 10−10. It is also preferred that the antisense molecules bind the target molecule with a kD less than

A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with kDs from the target molecule of less than 10−12 M. It is preferred that the aptamers bind the target molecule with a kD less than 10−6. It is more preferred that the aptamers bind the target molecule with a kD less than 10−8. It is also more preferred that the aptamers bind the target molecule with a kD less than 10−10. It is also preferred that the aptamers bind the target molecule with a kD less than 10−12. Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a kD with the target molecule at least 10 fold lower than the kD with a background binding molecule. It is more preferred that the aptamer have a kD with the target molecule at least 100 fold lower than the kD with a background binding molecule. It is more preferred that the aptamer have a kD with the target molecule at least 1000 fold lower than the kD with a background binding molecule. It is preferred that the aptamer have a kD with the target molecule at least 10000 fold lower than the kD with a background binding molecule. It is preferred when doing the comparison for a polypeptide for example, that the background molecule be a different polypeptide. For example, when determining the specificity of a promoter for detecting the Cox protein, or any other disclosed molecule aptamers, the background protein could be serum albumin. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a kD less than 10−6. It is more preferred that the triplex forming molecules bind with a kD less than 10−8. It is also more preferred that the triplex forming molecules bind the target molecule with a kD less than 10−10. It is also preferred that the triplex forming molecules bind the target molecule with a kD less than 10−12. Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target an RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan, Proc. Natl. Acad. Sci. USA 89:8006-8010 (1992); WO 93/22434 by Yale; WO 95/24489 by Yale; Yuan and Altman, EMBO J 14:159-168 (1995), and Carrara, Proc. Natl. Acad. Sci. (USA) 92:2627-2631 (1995)). Representative examples of how to make and use EGS molecules to facilitate cleavage of a variety of different target molecules can be found in the following non-limiting list of U.S. Pat. Nos. 5,168,053, 5,624,824, 5,683,873, 5,728,521, 5,869,248, and 5,877,162.

4. Delivery of the Vectors to Cells

The disclosed vectors can be delivered to the target cells in a variety of ways. The vector can be administered directly to cells in culture or injected systemically or locally into the body, whereupon the vector transduces through the cell membrane and into the cell's interior. Alternatively, the vectors can be delivered through electroporation, or through lipofection, or through calcium phosphate precipitation. The delivery mechanism chosen will depend in part on the type of cell targeted and whether the delivery is occurring for example in vivo or in vitro.

5. Nucleic Acids

There are a variety of molecules disclosed herein that are nucleic acid based, including for example the vectors described herein, as well as various functional nucleic acids, such as the vector comprising SEQ ID NO: 8, for example. The disclosed nucleic acids are made up of for example, nucleotides, nucleotide analogs, or nucleotide substitutes. Non-limiting examples of these and other molecules are discussed herein. It is understood that for example, when a vector is expressed in a cell that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenine-9-yl (A), cytosine-1-yl (C), guanine-9-yl (G), uracil-1-yl (U), and thymine-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide that contains some type of modification to either the base, sugar, or phosphate moieties. Modifications to the base moiety would include natural and synthetic modifications of A, C, G, and T/U as well as different purine or pyrimidine bases, such as uracil-5-yl, hypoxanthine-9-yl (I), and 2-aminoadenine-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and 0-6 substituted purines, including 2-aminopropyladenine, 5-propynyluracil and 5-propynylcytosine. 5-methylcytosine can increase the stability of duplex formation. Often time base modifications can be combined with for example a sugar modification, such as 2′-O-methoxyethyl, to achieve unique properties such as increased duplex stability. There are numerous such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyallcylamino, substituted silyl, an RNA cleaving group, a reporter group, an intercalator, a group for improving the pharmacolinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only contain a single modification but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen, Science, 1991, 254, 1497-1500).

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger, Proc. Natl. Acad. Sci. USA, 1989, 86, 6553-6556), cholic acid (Manoharan, Bioorg. Med. Chem. Let., 1994, 4, 1053-1060), a thioether, e.g., hexyl-S-tritylthiol (Manoharan, Ann. N. Y. Acad. Sci., 1992, 660, 306-309; Manoharan, Bioorg. Med. Chem. Let., 1993, 3, 2765-2770), a thiocholesterol (Oberhauser, Nucl. Acids Res., 1992, 20, 533-538), an aliphatic chain, e.g., dodecandiol or undecyl residues (Saison-Behmoaras, EMBO J., 1991, 10, 1111-1118; Kabanov, FEBS Lett., 1990, 259, 327-330; Svinarchuk, Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan, Tetrahedron Lett., 1995, 36, 3651-3654; Shea., Nucl. Acids Res., 1990, 18, 3777-3783), a polyamine or a polyethylene glycol chain (Manoharan, Nucleosides & Nucleotides, 1995, 14, 969-973), or adamantane acetic acid (Manoharan, Tetrahedron Lett., 1995, 36, 3651-3654), a palmityl moiety (Mishra, Biochim. Biophys. Acta, 1995, 1264, 229-237), or an octadecylamine or hexylamino-carbonyl-oxycholesterol moiety (Crooke, J. Pharmacol. Exp. Ther., 1996, 277, 923-937. Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences for the vectors. It is understood that the description related to these sequences is applicable to any sequence related thereto unless specifically indicated otherwise. Those of skill in the art understand how to resolve sequence discrepancies and differences and to adjust the compositions and methods relating to a particular sequence to other related sequences. Primers and/or probes can be designed for any sequence given the information disclosed herein and known in the art.

6. Antibodies

a) Antibodies Generally

The invention further provides antibodies to the reporter protein or reporter protein ligands for use in imaging. As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “antibody or fragments thereof” encompasses antibodies and hybrid antibodies, with dual or multiple antigen or epitope specificities, and fragments, such as scFv, sFv, F(ab′)2, Fab′, Fab and the like, including hybrid fragments. Thus, fragments of the antibodies that retain the ability to bind their specific antigens are provided. For example, fragments of antibodies which maintain binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane, Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits, Proc. Natl. Acad. Sci. USA, 90:2551-255 (1993); Jakobovits, Nature, 362:255-258 (1993); Bruggemann, Year in Immuno., 7:33 (1993)). Human antibodies can also be produced in phage display libraries (Hoogenboom, J. Mol. Biol., 227:381 (1991); Marks, J. Mol. Biol., 222:581 (1991)). The techniques of Cole and Boerner are also available for the preparation of human monoclonal antibodies (Cole, Monoclonal Antibodies and Cancer Therapy, Alan R. Liss, p. 77 (1985); Boerner, J. Immunol., 147(1):86-95 (1991)).

The human antibodies of the invention can be prepared using any technique. Examples of techniques for human monoclonal antibody production include those described by Cole (Monoclonal Antibodies and Cancer Therapy, Alan R. Liss, p. 77, 1985) and by Boerner (J. Immunol., 147(1):86-95, 1991). Human antibodies of the invention (and fragments thereof) can also be produced using phage display libraries (Hoogenboom, J. Mol. Biol., 227:381, 1991; Marks, J. Mol. Biol., 222:581, 1991).

The human antibodies of the invention can also be obtained from transgenic animals. For example, transgenic, mutant mice that are capable of producing a full repertoire of human antibodies, in response to immunization, have been described (see, e.g., Jakobovits, Proc. Natl. Acad. Sci. USA, 90:2551-255 (1993); Jakobovits, Nature, 362:255-258 (1993); Bruggermann, Year in Immunol. 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fc, Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones, Nature, 321:522-525 (1986), Reichmann, Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones, Nature, 321:522-525 (1986), Riechmann, Nature, 332:323-327 (1988), Verhoeyen, Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly), U.S. Pat. No. 5,565,332 (Hoogenboom), U.S. Pat. No. 5,721,367 (Kay), U.S. Pat. No. 5,837,243 (Deo), U.S. Pat. No. 5, 939,598 (Kucherlapati), U.S. Pat. No. 6,130,364 (Jakobovits), and U.S. Pat. No. 6,180,377 (Morgan).

b) Administration of Antibodies

Antibodies of the invention are preferably administered to a subject in a pharmaceutically acceptable carrier. Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of antibody being administered.

The antibodies can be administered to the subject, patient, or cell by injection (e.g., intravenous, intraperitoneal, subcutaneous, intramuscular), or by other methods such as infusion that ensure its delivery to the bloodstream in an effective form. Local or intravenous injection is preferred. Furthermore, ex vivo administration can be used wherein cells or tissues are isolated, treated, and returned to the subject to be treated.

Effective dosages and schedules for administering the antibodies may be determined empirically, and making such determinations is within the skill in the art. Those skilled in the art will understand that the dosage of antibodies that must be administered will vary depending on, for example, the subject that will receive the antibody, the route of administration, the particular type of antibody used and other drugs being administered. Guidance in selecting appropriate doses for antibodies is found in the literature on therapeutic uses of antibodies, e.g., Handbook of Monoclonal Antibodies, Ferrone, eds., Noges Publications, Park Ridge, N.J., (1985) ch. 22 and pp. 303-357; Smith, Antibodies in Human Diagnosis and Therapy, Haber, eds., Raven Press, New York (1977) pp. 365-389. A typical daily dosage of the antibody used alone might range from about 1 μ/g/kg to up to 100 mg/kg of body weight or more per day, depending on the factors mentioned above.

Antibodies disclosed herein can also be used to detect various compounds of the invention. Such antibodies can be used for research and clinical purposes.

7. Pharmaceutical Carriers/delivery of Pharmaceutical Products

a) Administration

The compositions, including the vectors, of the invention, can be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions maybe administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials maybe in solution or suspension (for example, incorporated into microparticles, liposomes, or cells). These may be targeted to a particular cell type via antibodies, receptors, or receptor ligands. The following references are examples of the use of this technology to target specific proteins to tumor tissue (Senter, Bioconjugate Chem., 2:447-451, (1991); Bagshawe, K. D., Br. J. Cancer, 60:275-281, (1989); Bagshawe, Br. J. Cancer, 58:700-703, (1988); Senter, Bioconjugate Chem., 4:3-9, (1993); Battelli, Cancer Immunol. Immunother., 35:421-425, (1992); Pietersz and McKenzie, Immunolog. Reviews, 129:57-80, (1992); and Roffler, Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue Hughes, Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

b) Liposomes

Liposomes are vesicles comprised of one or more concentrically ordered lipid bilayers which encapsulate an aqueous phase. They are normally not leaky, but can become leaky if a hole or pore occurs in the membrane, if the membrane is dissolved or degrades, or if the membrane temperature is increased to the phase transition temperature. Current methods of drug delivery via liposomes require that the liposome carrier ultimately become permeable and release the encapsulated drug at the target site. This can be accomplished, for example, in a passive manner wherein the liposome bilayer degrades over time through the action of various agents in the body. Every liposome composition will have a characteristic half-life in the circulation or at other sites in the body and, thus, by controlling the half-life of the liposome composition, the rate at which the bilayer degrades can be somewhat regulated.

In contrast to passive drug release, active drug release involves using an agent to induce a permeability change in the liposome vesicle. Liposome membranes can be constructed so that they become destabilized when the environment becomes acidic near the liposome membrane (see, e.g., Proc. Natl. Acad. Sci. USA 84:7851 (1987); Biochemistry 28:908 (1989), which is hereby incorporated by reference in its entirety). When liposomes are endocytosed by a target cell, for example, they can be routed to acidic endosomes which will destabilize the liposome and result in drug release.

Alternatively, the liposome membrane can be chemically modified such that an enzyme is placed as a coating on the membrane which slowly destabilizes the liposome. Since control of drug release depends on the concentration of enzyme initially placed in the membrane, there is no real effective way to modulate or alter drug release to achieve “on demand” drug delivery. The same problem exists for pH-sensitive liposomes in that as soon as the liposome vesicle comes into contact with a target cell, it will be engulfed and a drop in pH will lead to drug release. This liposome delivery system can also be made to target B cells by incorporating into the liposome structure a ligand having an affinity for B cell-specific receptors.

Compositions including the liposomes in a pharmaceutically acceptable carrier are also contemplated.

c) Transdermal Delivery Devices

Transdermal delivery devices have been employed for delivery of low molecular weight proteins by using lipid-based compositions (i.e., in the form of a patch) in combination with sonophoresis. However, as reported in U.S. Pat. No. 6,041,253 to Ellinwood, Jr. et al., which is hereby incorporated by reference in its entirety, transdermal delivery can be further enhanced by the application of an electric field, for example, by ionophoresis or electroporation. Using low frequency ultrasound which induces cavitation of the lipid layers of the stratum corneum, higher transdermal fluxes, rapid control of transdermal fluxes, and drug delivery at lower ultrasound intensities can be achieved. Still further enhancement can be obtained using a combination of chemical enhancers and/or magnetic field along with the electric field and ultrasound.

d) Vectors

The nucleic acid can also be a viral vector comprising a nucleic acid encoding a reporter, as described herein. One skilled in the art will appreciate that the viral vector utilized can comprise any viral vector amenable to delivery to an area of inflammation, such as the lungs, the kidneys, the liver, or to the site of a tumor. For example, the viral vector can be a recombinant adenovirus vector, an adeno-associated viral vector, a lentiviral vector, a pseudotyped retroviral vector, a vaccinia vector, an alphavirus vector, or any other viral vector known in the art or described throughout.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

As noted above, the viral vector of this invention can be a retrovirus. A retrovirus is an animal virus belonging to the virus family of Retroviridae, including 10 any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference. The retrovirus of this invention can be in the Oncovirinae subfamily of retroviruses, such as HTLV-I or HTLV-II (human T-cell leukemia virus type I and type II, respectively).

Additionally, the retrovirus can be in the Lentivirinae subfamily of retroviruses, such as HIV-1, HIV-II, SIV, FIV, EIAV and CAEV (human immunodeficiency virus type I, human immunodeficiency virus type II, simian immunodeficiency virus, feline immunodeficiency virus, equine infectious anemia virus, and caprine arthritis-encephalitis virus, respectfully).

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

Adenovirus vectors are disclosed throughout. In an embodiment where the viral vector is an adenovirus, the nucleic acid can comprise an entire wild-type adenoviral genome or a mutant thereof, or a construct wherein the only adenoviral sequences present are those which enable the nucleic acid to be packaged into an adenovirus particle, or any variation thereof. The term “adenovirus” refers to replication incompetent vectors as well as replication competent and conditionally replication competent. Packageable lengths of nucleic acids are known in the art. The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

Disclosed herein are adenoviral vectors and AAV vectors containing an insertion in a hypervariable region. For example, the insertion is placed into the HVR2 or HVR5 region of the adenoviral vector. Insertions into other hypervariable regions are contemplated.

Optionally, the insertion comprises a nucleotide sequence encoding one or more repeats of a nucleic acid that encodes ED. The nucleic acid may further encode a His tag, including, for example, a 3-, 4-, 5-, 6-, 7-, 8-, 9-, 10-His tag. The His tag can be before, after, or between the repeated ED1 encoding regions. The invention also provides the nucleotide sequence comprising the insert. The insert can comprise a nucleotide sequence that encodes the amino acid sequence of SEQ ID NO:9. Thus, provided herein is the nucleotide sequence of SEQ ID NO:8. Also provided are nucleotide sequences with at least about 80%, 85%, 90%, 95% identity, or any identity in between those values, as compared to SEQ ID NO:8. Also provided is a nucleic acid that selectively hybridizes to the nucleotide sequence of SEQ ID NO:8 at stringent conditions. Also provided is a vector comprising the nucleic acid insert operably linked to an expression control sequence and a cell comprising the vector.

This adenoviral genome can be coupled with any desired nucleic acid encoding a reporter, as described herein, as well as a promoter, such that the adenoviral genome, when packaged into an adenovirus particle, also packages the nucleic acid insert. One skilled in the art will appreciate that the nucleic acid insert combined with the adenoviral nucleic acid will be of a total nucleic acid length that will allow the total nucleic acid to be packaged into an adenovirus particle.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (s) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The vectors of the present invention thus provide DNA molecules which are capable of integration into a mammalian chromosome without substantial toxicity.

Molecular genetic experiments with large human herpes viruses have provided a means whereby large heterologous DNA fragments can be cloned, propagated and established in cells permissive for infection with herpes viruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable. The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

Either administration of the vector in vivo or administration of in vitro cells can be utilized to monitor inflammation in accordance with the presently claimed invention.

e) Protein Depots

Implantable or injectable protein depot compositions can also be employed, providing long-term delivery of, e.g., the reporter and promoter nucleic acids. For example, U.S. Pat. No. 6,331,311 to Brodbeck, which is hereby incorporated by reference in its entirety, reports an injectable depot gel composition which includes a biocompatible polymer, a solvent that dissolves the polymer and forms a viscous gel, and an emulsifying agent in the form of a dispersed droplet phase in the viscous gel. Upon injection, such a gel composition can provide a relatively continuous rate of dispersion of the agent to be delivered, thereby avoiding an initial burst of the agent to be delivered.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

f) Pharmaceutically Acceptable Carriers

Disclosed are compositions comprising the vector and a pharmaceutical carrier. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including opthamalically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

g) Diagnostic Uses

The dosage ranges for the administration of the compositions are those large enough to produce the desired effect of inflammation monitoring. The dosage should not be so large as to cause adverse side effects, such as unwanted cross-reactions, anaphylactic reactions, and the like. Generally, the dosage will vary with the age, condition, sex and extent of the inflammation in the patient and can be determined by one of skill in the art. The dosage can be adjusted by the individual physician in the event of any contraindications. Dosage can vary, and can be administered in one or more dose administrations daily, for one or several days. While individual needs vary, determination of optimal ranges of effective amounts of the vector is within the skill of the art. Typical dosages comprise about 0.01 to about 100 mg/kg·body wt. The preferred dosages comprise about 0.1 to about 100 mg/kg·body wt. The most preferred dosages comprise about 1 to about 100 mg/kg·body wt.

Other vectors which do not have a specific inflammation monitoring function, but which may be used for tracking changes within cellular chromosomes or for the delivery of diagnostic tools for example can be delivered in ways similar to those described for the pharmaceutical products.

8. Kits

Disclosed herein are kits that comprise vectors that can be used in practicing the methods disclosed herein. For example, a kit can comprise a vector for monitoring inflammation, including a reporter and a promoter. The kit can further comprise instructions, and in vivo or in vitro monitoring equipment or supplies. The kits can include any reagent or combination of reagent discussed herein or that would be understood to be required or beneficial in the practice of the disclosed methods.

9. Compositions with Similar Functions

It is understood that the compositions disclosed herein have certain functions, for example, the reporter nucleic acid allows for imaging of inflammation. Disclosed herein are certain structural requirements for performing the disclosed functions, and it is understood that there are a variety of structures which can perform the same function which are related to the disclosed structures, and that these structures will ultimately achieve the same result, for example, monitoring inflammation as previously described.

10. Genetically Modified Animals

Disclosed are animals that comprise the vector of the invention. Also provided are animals such as the mouse that are transgenic, wherein the animal comprises a reporter nucleic acid operably linked to a promoter nucleic acid, wherein said reporter nucleic acid is expressed under conditions of inflammation. These transgenic animals can be made in many ways, by for example, the method of Yull (J Histochem. & Cytochem, 51(6):741-749). For example, the mice can be engineered to carry a promoter, such as the Cox-2L promoter, driving expression of a reporter such as luciferase.

The disclosed animals can be used in a variety of ways. For example, they can be used as tools to study inflammation in vivo. The animal can be exposed to in vivo monitoring as described herein to monitor inflammation. The disclosed animals can be used for drug discovery and for drug validation. Substances that are known or suspected of causing inflammation can be administered to the animal, and the affects thereof monitored. Alternatively, substances known or suspected of treating or ameliorating the symptoms of inflammation can be administered to the animal, and the affects thereof monitored. Combinations of the above can also be used to monitor the cause and effect relationship of various drug candidates. The organs of the animal can also be used ex vivo to monitor inflammation and the response to drugs and/or various treatments. The disclosed animals can also be used to as reagents to produce other beneficial transgenic animals, by for example, breeding the disclosed transgenic animals with other transgenic animals, producing double or even multiple transgenics. These animals are useful as model systems for drug discovery and validation.

11. Cell Lines

Disclosed are cell lines comprising a vector, said vector comprising a reporter nucleic acid operably linked to a promoter nucleic acid, wherein said reporter nucleic acid is expressed under conditions of inflammation. For example, the cell line can contain a promoter, such as a Cox-2 promoter, and a reporter, such as luciferase.

The disclosed cell lines can be used in a variety of ways. For example, they can be used as tools to study inflammation in vitro. The cell line can be exposed to in vitro monitoring as described herein to monitor inflammation. Alternatively, cells of the cell line can be administered to a test animal and in vivo monitoring can be used as described herein. The cell line can be used for drug discovery and for drug validation. Substances that are known or suspected of causing inflammation can be administered to the cell line, and the affects thereof monitored. Alternatively, substances known or suspected of treating or ameliorating the symptoms of inflammation can be administered to the cell line, and the affects thereof monitored. Combinations of the above can also be used to monitor the cause and effect relationship of various drug candidates. The disclosed cell lines can also be used as reagents to produce other beneficial cell lines, by for example, allowing the cell lines to multiply. These cell lines are useful as model systems for drug discovery and validation.

The present invention is more particularly described in the following examples, which are intended as illustrative only since numerous modifications and variations therein will be apparent to those skilled in the art.

Although the present process has been described with reference to specific details of certain embodiments thereof, it is not intended that such details should be regarded as limitations upon the scope of the invention except as and to the extent that they are included in the accompanying claims.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### D. EXAMPLES

### 1. Example 1

### The role of Complement in Liver Transduction

To directly address the role of complement in liver transduction, studies were performed using wild type mice versus mutant mice unable to make complement component C3. By repeated bioluminescence imaging of living mice, liver luciferase expression was assessed following intravenous delivery of the Ad vector.

In FIG. 4, representative images are presented that are captured from mice that received the lowest dose (2.3×10⁹ v.p.) of Ad5Luc1. Each image (1-min acquisition) was collected on day 13 after Ad5Luc1 delivery; the pseudocolor overlay represents the intensity of light emission, and thus the level of luciferase expression. Overall, wild type mice showed 12.7-fold greater liver luciferase expression than C3^{-/-} mice at this time point, and the absolute difference was statistically significant (p<0.05, ANOVA).

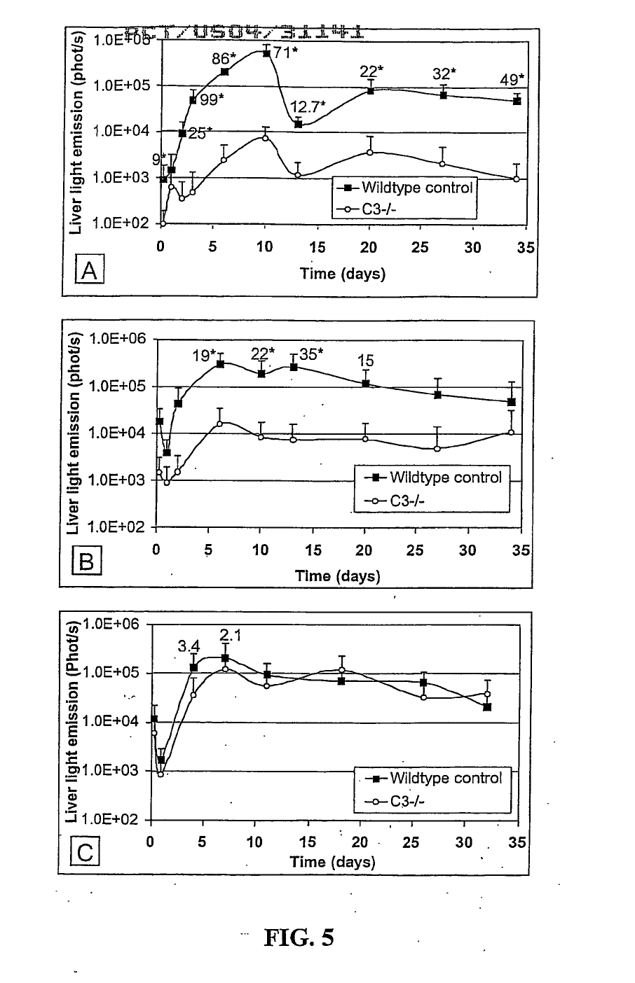

With all 3 doses of Ad5Luc1, peak liver luciferase expression in both kinds of mice was detected on day 6-10 (FIG. 5). Maximal luciferase expression ranged from 10- to 100-times greater than that observed 1-2 d after vector administration. Wild type mice always showed higher liver luciferase expression, but the absolute difference between wild type and C3^{-/-} mice was diminished as the dose of Ad5Luc1 was increased. For example, liver luciferase expression 3 days after injection of 2.3×10⁹ v.p. was 99-fold higher in wild type mice compared to C3^{-/-} mice (FIG. 5A). For mice injected with 4.0×10⁹ v.p. (FIG. 5B), wild type mice showed 35-fold higher liver luciferase expression compared to C3^{-/-} mice. For the highest Ad5Luc1 dose (1.3×10¹⁰ v.p.), the maximal difference between the two groups was 3.4-fold (FIG. 5C). For the C3^{-/-} mice in isolation, significantly greater luciferase expression in the liver was observed with increasing Ad5Luc1 vector dose. In contrast, the control mice with an intact complement system did not show greater luciferase expression with increasing Ad5Luc1 dose.

If complement activation leads to opsonization of the vector and results in Ad5Luc1 clearance via the reticuloendothelial system, then it would be expected that C3^{-/-} mice have higher levels of luciferase expression in the liver compared to wild type mice, but this was not observed at any of the doses tested. Rather, complement appeared to facilitate liver transduction, i.e. C3^{-/-} mice showed lower luciferase expression than wild type mice. The facilitation effect was overcome if high numbers of the vector were injected, thus C3^{-/-} and wild type mice showed similar liver luciferase expression after administration of 1.3×10¹⁰ Ad5Luc1. Importantly, none of the mice used in the present study were previously exposed to Ad vectors, so the complement-dependent effect was likely antibody-independent.

The finding that absence of C3 was associated with lower transduction of the liver points to an important role of complement in the transduction process (Fernie-King B, Ann Rheum Dis 2002; 61 Suppl 2: ii8-12; Fernie-King B A, Infect Immun 2002; 70: 4908-4916; Lachmann P J. Proc Natl Acad Sci U S A 2002; 99: 8461-8462; Duthy T G Infect Immun 2002; 70: 5604-5611; Meri T Infect Immun 2002; 70: 5185-5192; Meri T J Infect Dis 2002; 185: 1786-1793; Stoiber et al. Vaccine 2003, 21 Supp 2: S77-82).

It was found that inhibition of complement can be a valid approach to overcome the liver's propensity to remove systemically administered Ad designed to target other organs and tissues. Complement depletion has also been found to improve systemic delivery of replication-conditional Herpes vectors to brain tumors (Ikeda Nat Med 1999, 5:881-7). Inhibition of complement activation has the added benefit of decreasing the humoral and cell mediated immune response to virus (Suresh M. J Immunol 2003 170:788-94). Ad vectors can display complement regulatory proteins on their surface, or other surface proteins capable of binding negative regulators of complement activation in host blood. Sites of incorporation of these proteins in the Ad include the Ad fiber or knob, pIX, a site demonstrated for genetic addition of peptides (Dmitriev I P, J Virol 2002, 76:6893-6899). A linker site (poly GGGGS, SEQ ID NO: 11) between the FF chimera and retargeting ligands is a second potential site (Krasnykh J Virol 2001, 75:4176-4183). A third site for incorporation is the hexon protein, a major structural protein for the Ad vector. In this manner, negative regulators of complement activation are present on the surfaces of the Ad vector. Complement activation is thereby reduced, minimizing undesired toxicities and/or improving targeting outcomes.

### 2. Example 2

### Bioluminescence Imaging in the Liver and Lungs

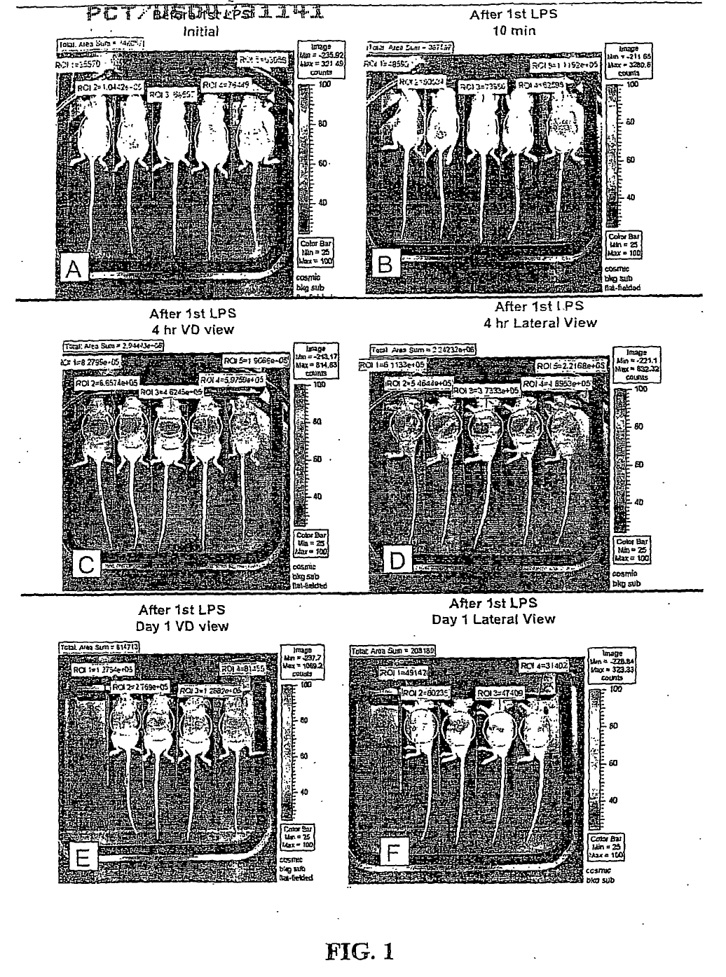

Bioluminescence imaging was applied to assess luciferase expression in liver; low doses of LPS induces luciferase expression. Five mice were i.v. injected with a replication incompetent serotype 5 Ad encoding luciferase (2×10⁸ pfu), under control of the cox2L promoter. Luciferase expression was detected by measuring light emission from the mice using a CCD camera (Xenogen IVIS system) at 15 min after injection of 2 mg of luciferin i.p. FIG. 1 presents overlays of mice images with pseudocolor images in which the different colors represent the intensity of light emission from the mouse. The relative photons emitted in an area of the mouse was determined by region of interest analyses. For all,mice, the luciferase expression in liver was extremely low, essentially undetectable by 3 days after dosing with Ad-cox2L-Luc (FIG. 1A). At 10 minutes after injection of 2 micrograms of LPS, no induction of luciferase expression was detectable. However, by 4 hr after injection of LPS the induced luciferase expression was detected by imaging approximately 12-fold over background signal (FIG. 1C-D). The expression was transient, and was reduced to nearly background signal by 24 h after LPS injection (FIG. 1E-F).

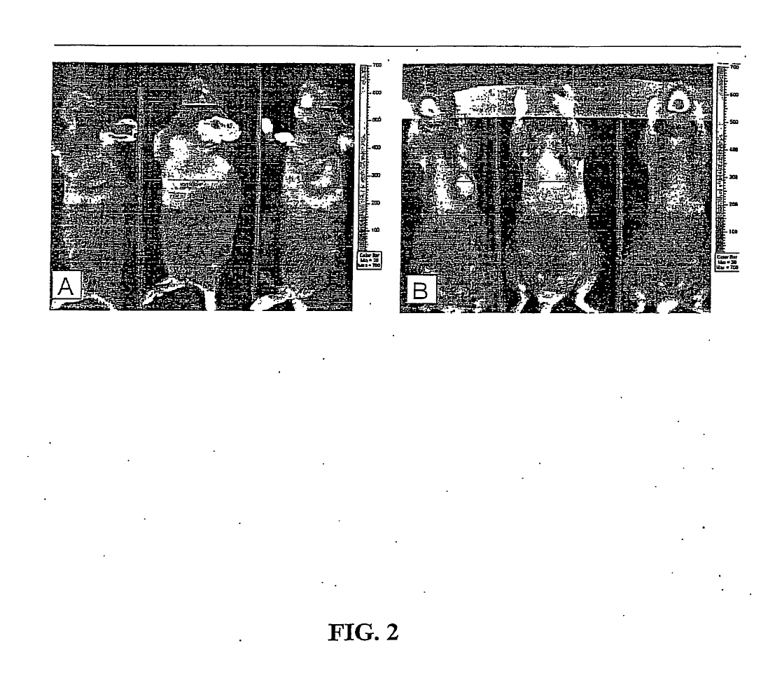

Bioluminescence imaging detects luciferase expression in lung. As shown in FIG. 2, luciferase expression in lung was detected at 10 days after intratracheal (i.t.) delivery of an Ad encoding luciferase (driven by CMV, 5×10⁸ pfu). Two views are shown in FIG. 2, demonstrating the capacity of the bioluminescence system for detection of lung luciferase expression.

### 3. Example 3

### Sensitivity and Specificity of the cox2L-luciferase Reporter Construct and in vivo Monitoring in Mouse Lung

Already-prepared plasmids and Ad vectors encoding the firefly luciferase gene under control of the CMV promoter, or the cox2L promoter are used. Initial experiments are conducted with two groups of immune competent BL/6 mice (5/group) using two intratracheal doses (1×10⁸ and 1×10⁹ pfu) of the Ad vectors encoding luciferase. These experiments establish basal luciferase expression levels for both CMV and cox2L constructs. Subsequent experiments use LPS (for example, 3 mg I.P.) to induce lung luciferase expression driven by cox2L. LPS for the purpose of inducing lung inflammation and pulmonary administration of plasmid based detection vectors, is tested as described previously (Wright, M. Amer J Res Crit Car Med 165:A41, 2002). Bioluminescence imaging is used to follow the changes in luciferase expression over time following LPS administration. Repeated dosing of LPS is conducted as needed. Anti-inflammation drugs (corticosteroids and NSAIDs) can additionally be evaluated to reduce the levels of luciferase expression. For example, dexamethasone can be administered as a standard anti-inflammatory regimen in mice. All experiments include a control group in which lung luciferase expression is driven by CMV, and are therefore unlikely to be altered by drug treatment.

A Xenogen IVIS system was used for bioluminescence imaging. This system has high sensitivity, and can image 5 mice at the same time. Rodents are injected with 2 mg of luciferin, 15 min prior to imaging. Light-based imaging signal can be quantified. Appropriate calibration standards are imaged in established positions to insure a constant detection signal under well defined conditions. For bioluminescence, a stable calibration light-source provided by Xenogen is used. For both light-based methods the intensity of signal per pixel is determined using region of interest analyses. Tissues that are imaged in vivo are removed and measured independently. A Victor2 plate reader is used for in vitro luciferase measurements of these removed tissues.

### 4. Example 4

### Cox2L-luciferase to Monitor Inflammation in a Mouse Model of Cystic Fibrosis

The CFTR knockout mouse model of cystic fibrosis on a congenic (C57B1/6) background is used. This CF strain exhibits patchy pathologic changes consistent with small airway disease that includes accumulation of inflammatory cells (Kent G, J Clin Invest 100:3060-9, 1997). The pathology intensifies with age, and interstitial wall thickening and loss of alveolar architecture occurs. Initially, 10 weanling mice are dosed intratracheally with reporter constructs. The mice are imaged 3× per week (bioluminescence) to monitor inflammation. A comparison is made with non CF littermates (matched for age) and with non-CF mice of the same genetic background matched for weight. If inflammation is detected, half of the mice are treated with either corticosteroids or NSAIDs (see above). All mice are monitored for several weeks. Bioluminescence studies of luciferase under cox2L regulation are conducted as described above. A nutrient liquid diet is used to prolong survival in the CF animals. As needed, X-ray CT studies and lung perfusion studies using Tc-99m-MAA (macroaggregated albumin) and SPECT are conducted.

### 5. Example 5

### Evaluation of a Genetic Reporter Construct (cox2-hSSTr2) for Monitoring of Inflammation

Tc-99m labeled Ad encoding hSSTr2 (driven by CMV or cox2L) according to previously described methods (Zinn K R Radiology 223:417-25, 2002; Zinn K R J Ntur 129:181-7, 1999; Zinn K R Arthritis Rheum 42:641-9, 1999) are instilled in the lung of CFTR deficient mice. SPECT imaging studies are conducted to verify the location of Ad delivery. Subsequently, the mice are imaged for hSSTr2 expression using the hSSTr2-avid peptide. X-ray CT studies are conducted simultaneously for anatomical localization, and to determine potential association between anatomic changes that occur over time in relationship to cox2L-driven hSSTr2 expression.

Regarding gamma camera imaging, a SPECT/CT system for 3-dimensional rodent imaging at 1 mm spatial resolution is used. The SPECT/CT system requires approx. 30 minutes to collect images at ˜1 mm resolution. The tomographic images are reconstructed using an algebraic reconstruction technique algorithm. Coronal tomographic slices (1 mm each) are displayed and images evaluated by manual region of interest (ROI) analyses. The total number of voxels within the ROI in some cases can be used to determine the volume of a particular target tissue, since each voxel corresponds to 1 mm³. Pulmonary perfusion is evaluated by Tc-99m-MAA (macroaggregated albumin) while X-ray CT is applied to evaluate changes in lung anatomy. These changes include bronchiectasis, atelectasis, mucous plugging, and bronchial wall thickening (Oikonomou Eur Radiol 12:2229-35, 2002; Tiddens H A Pediatr Pulmonol 34:228-31, 2002) (FIG. 6).

### 6. Example 6

### Intravenous Injection of Ad5 Vectors Delivering Reporter Genes to Tumors

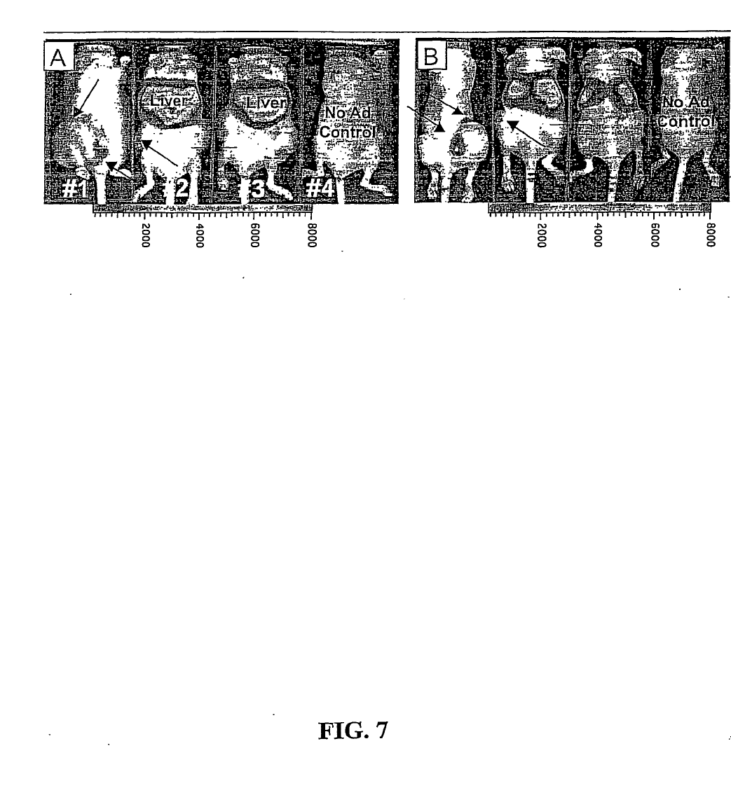

Tumor luciferase expression was detected within 4 d following intravenous injection of a replication incompetent Ad encoding luciferase (Ad5Luc1). FIG. 7 presents images showing tumor targeting from a representative experiment with a replication incompetent Ad5 vector (normal fiber structure) that was i.v. injected (1×10⁹ pfu/mouse) in nude mice bearing A-427 s.c. tumors. The bioluminescence images are pseudocolor images, with color representing levels of light emission. The induced luciferase expression in the tumors was consistent but the level varied, as did the level of luciferase expression in liver. This particular Ad vector was not specifically targeted to tumor, and had the normal CAR and integrin binding motifs.

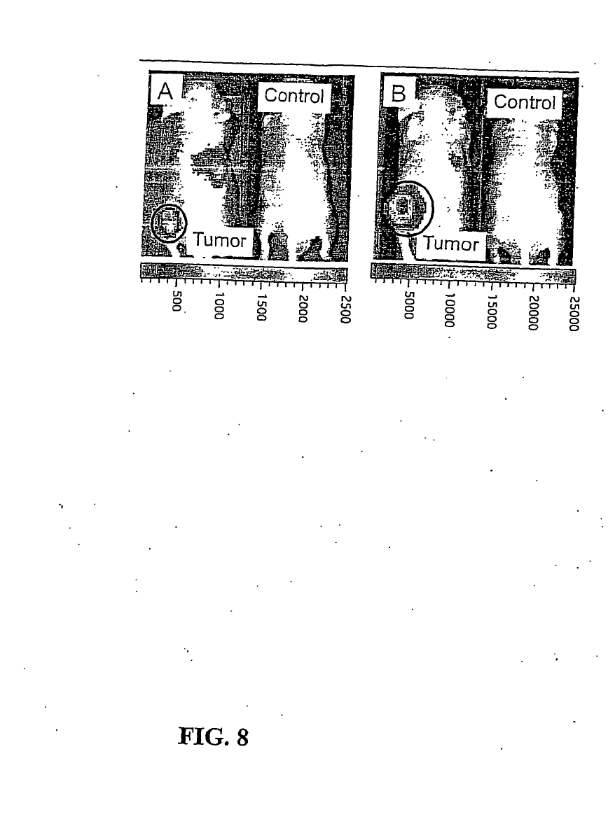

Increasing levels of luciferase expression were detected in subcutaneous breast tumors following intravenous injection of a replication competent Ad5 vector encoding luciferase (Ad5Luc3). FIG. 8 presents representative images of experiments conducted with s.c. human xenograft breast tumors (MB468) in nude mice. After establishing the s.c. tumors (10 weeks after 4×10⁶ cells/s.c. site), half of the mice were i.v. injected with Ad5Luc3 (2×10¹⁰ particles/mouse); the other half did not receive Ad vector. Initial images showed primarily liver expression in Ad-injected mice, but over time the liver expression was decreased while tumor expression increased. As shown in FIG. 8A, tumor expression of luciferase (left side) was detected by 14 d; liver luciferase expression was minimal at this time. The level of luciferase expression increased in the same tumors at 21 d (FIG. 8B). Note the difference in scale for FIGS. 8A and 8B; both images were collected for 5 min using the Xenogen system. As expected, no luciferase expression was detected in mice that did not receive the Ad vector.

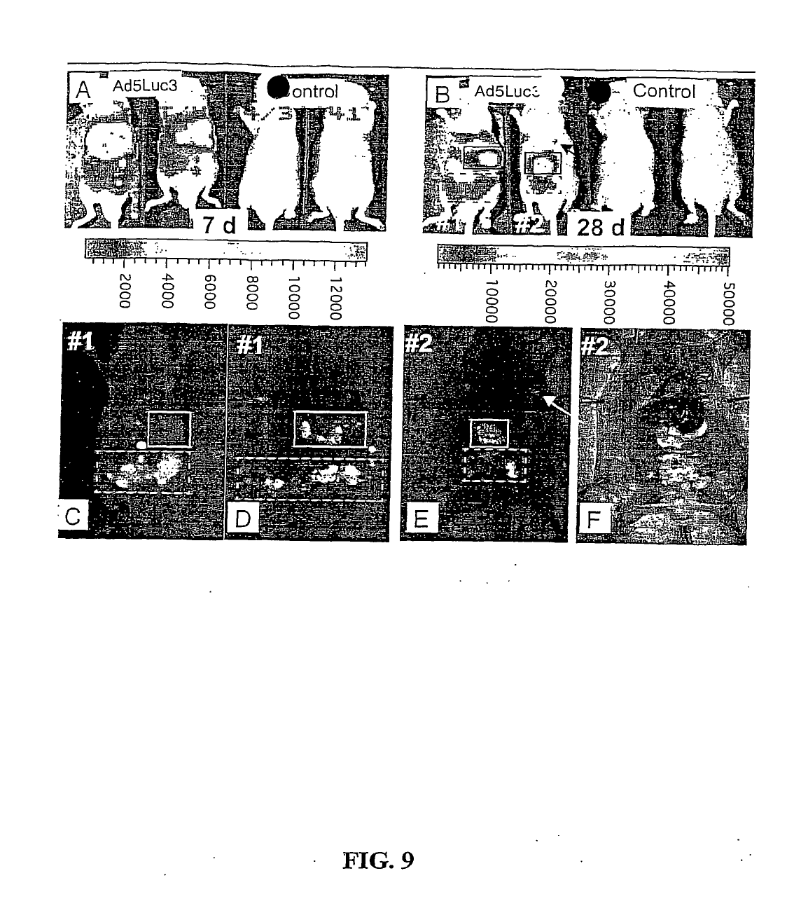

High levels of luciferase expression were detected in GFP-positive intraperitoneal xenograft prostate tumors following intravenous injection of a replication competent Ad5Luc3. GFP-positive PC3 cells (4×10⁶ cells/mouse) were implanted in the peritoneal cavity of nude mice (n=4). After 24 h, the replication competent Ad5Luc3 was i.v. injected in 2 mice (1×10¹⁰ particles/mouse). As shown in FIG. 9A, the i.v. injection of the Ad5Luc3 resulted in expression of luciferase in the peritoneal xenografts after 7 d. By 28 d the luciferase signal in tumor was significantly higher, while liver signal decreased. Mice #1 and #2 (from 9B) were imaged with the fluorescent stereomicroscope to correlate the in vivo location of luciferase expression with the location of the intrinsically GFP-positive tumors. As shown from the images in C-F, the correlation was excellent. One strong area of luciferase signal in mouse #1 (FIG. 9B, black rectangle-solid line) was not detected by the GFP imaging of the intact animal (FIG. 9C, white rectangle-solid line) due to the fact the GFP-positive tumor was located in a deeper abdominal area. However, when the mouse was opened it was readily apparent that the GFP-positive tumor was present at that location (FIG. 9D, white rectangle-solid line). This difference is explained by the fact that light emission from the luciferase enzymatic reaction is more penetrating through tissue due to its broader wavelength emission (including near infrared wavelengths) as compared with the wavelength of GFP emission (510+15 nm), and the further requirement that fluorescence imaging requires excitation light (485 nm) to reach the tumor, while this is not required for luciferase imaging.

These series of experiments validated that CAR and integrin mediated infection of human tumors could be achieved following i.v. delivery of the Ad vector to tumors. It was also established that viral replication increased the luciferase imaging signal, and thereby demonstrated the potential for conditionally-replicative vectors for i.v. injection.

### 7. Example 7

### Ad5 Vectors Containing Fiber-fibritin (FF) Chimeras with Fused Targeting Ligands Showed Desirable Characteristics for Tumor Targeting

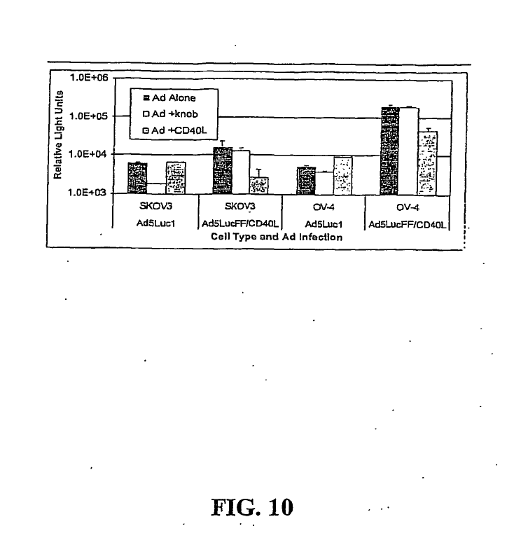

CAR deficient cell lines positive for CD40 showed increased transduction with Ad5LucFF/CD40L as compared with Ad5Luc1. A number of human cancers are positive for CD40 expression, making this receptor a viable candidate for in vivo targeting. As shown in FIG. 10, higher levels of luciferase were observed with infection of SKOV3 and OV-4 cells with Ad5LucFF/CD40L, as compared with Ad5Luc1. These data are supportive of using the fiber-fibritin replacement platform in combination with ligands (CD40L) that target CD40-positive tumor cells, and CD40-positive tumor endothelium.

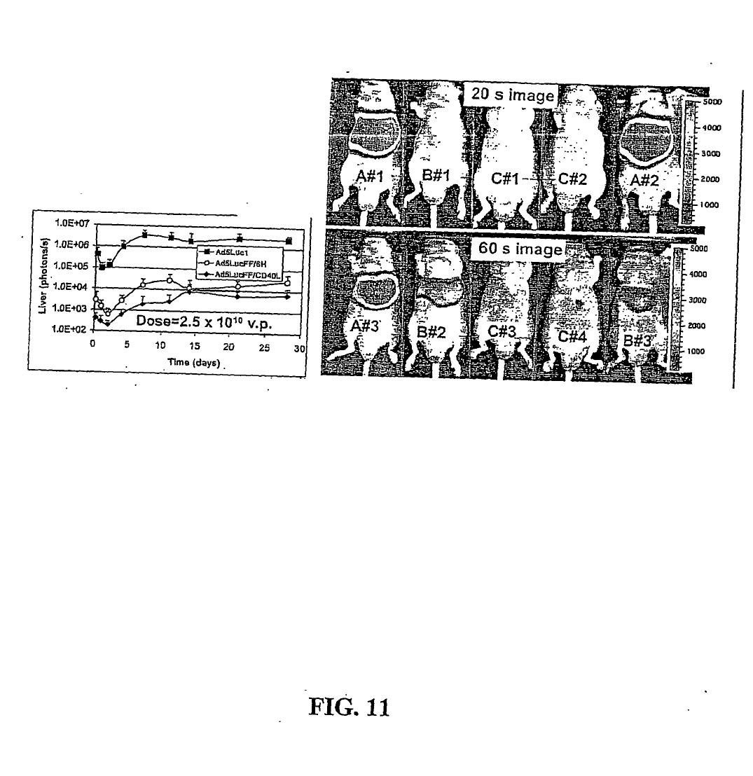

Liver luciferase expression was greater than 100-fold reduced with FF-containing Ad vectors as compared with Ad5luc1 (normal fiber). These results are presented in FIG. 11, for three Ad vectors that were i.v. injected (2.5×10¹⁰ particles/dose). Both FF-containing Ad vectors that were evaluated showed greater than 100-fold reduction in luciferase expression in liver, as compared with Ad5Luc1 (normal fiber). Longer exposure times were necessary to detect the liver luciferase expression in mice infected with the FF-containing Ad vectors. Note the first imaging time point on the graph was 6 h after Ad injection.

Intravenous injection of a Ad5LucFF/CD40L resulted in specific infection of CD40-positive ovarian tumor xenografts. As shown in FIG. 12, the i.v. injection of replication incompetent Ad5LucFF/CD40L (9×10¹⁰ particles/dose) resulted in luciferase expression in i.p. SKOV3 ovarian tumors at 5 d after dosing (600 s images). A similar dose of untargeted, replication incompetent Ad5Luc1 did not result in luciferase expression in the i.p. ovarian tumors.

These data support the use of Ad with FF chimeras as retargeting platforms. There was 100-fold lower liver transduction compared with Ad containing normal fibers. In addition, i.v. injection of Ad5LucFF/CD40L resulted in luciferase expression in CD40-positive SKOV3 tumor xenografts, while injection with Ad5Luc1 under similar conditions did not.

Summary of findings with respect to i.v. injection of Ad vectors: based on ˜10 experiments with i.v. injected Ad vectors, the following general findings are reported. (i) The time required to accomplish the i.v. injection ultimately influenced the level of liver transgene expression. Short, nearly immediate i.v. injections showed lower liver luciferase expression, while more prolonged i.v. injections resulted in higher liver luciferase expression. (ii) Images collected at 6 h after i.v. injections of the vectors showed higher levels of liver luciferase expression, as compared with images at 24-48 hours. This can indicate infection of a particular liver cell type (e.g. Kupffer cells) that is cleared.

### 8. Example 8

### Specific Tumor Targets in the Immune Competent Rat Model

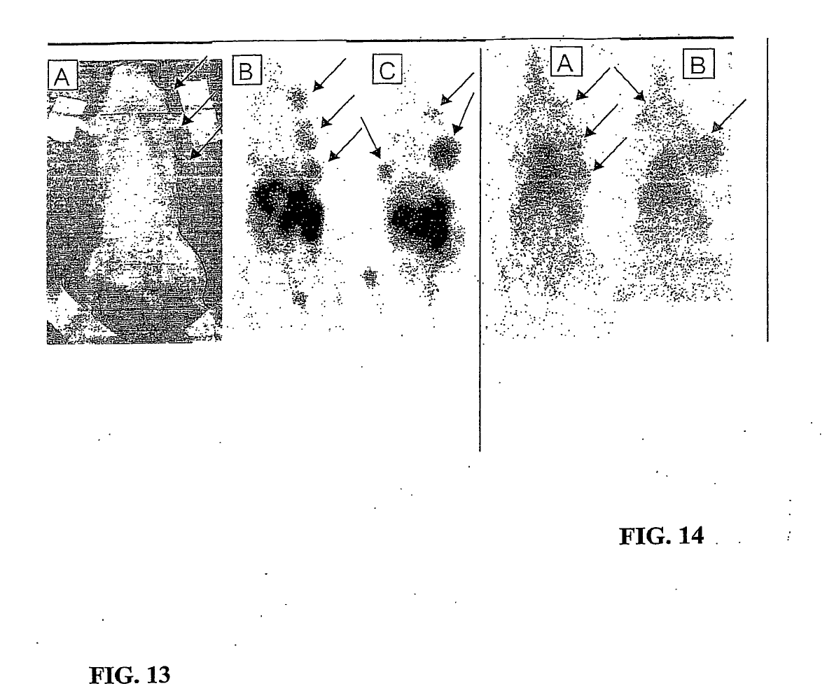

Specific tumor targets were identified in the immune competent rat model with MNU-induced mammary adenocarcinoma. MNU-induced mammary tumors in immune competent rats are positive for somatostatin receptor. As shown in FIG. 13, high uptake of Tc-99m-P2045 has been demonstrated, a somatostatin receptor binding peptide at 5 h after injection. It was known this peptide binds to both mouse and rat somatostatin receptors, besides its affinity for the human receptor. Many human tumors are also positive for somatostatin receptors. Therefore, this receptor is used for targeting in the rat model, using a somatostatin-avid peptide incorporated in the fiber-fibritin structure.

MNU-induced mammary tumors showed binding of an antibody specific for the tumor endothelium. Tumor targeting of an antibody that binds rat tumor endothelium specifically was shown (FIG. 14). Up to 25% dose/g in a rat lung tumor model were measured, within 30 min following i.v. injection of the antibody.

### 9. Example 9

### Innate and Systemic Immune Response to the Ad5 Vector

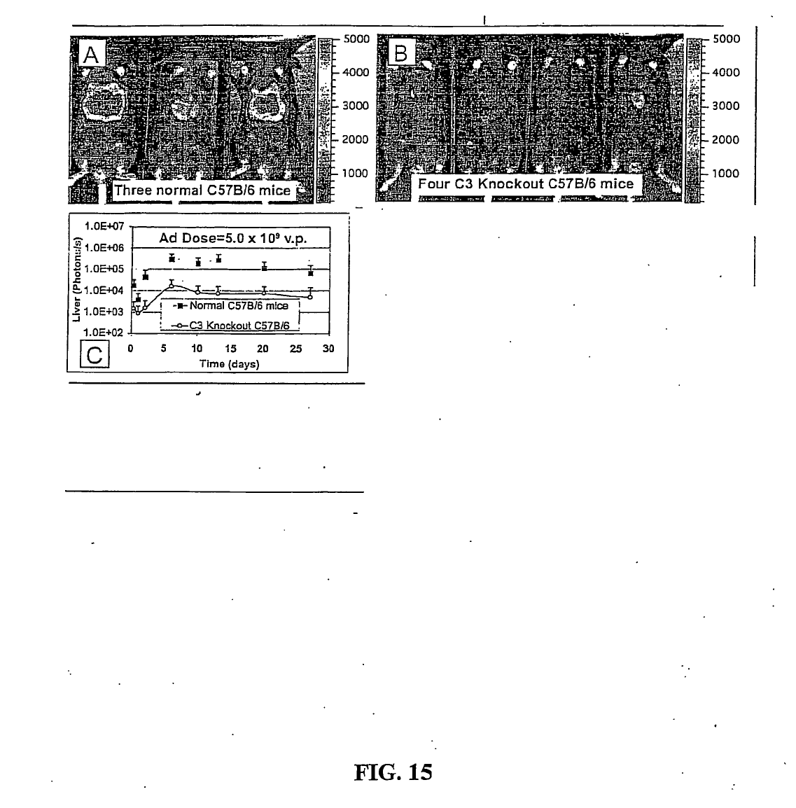

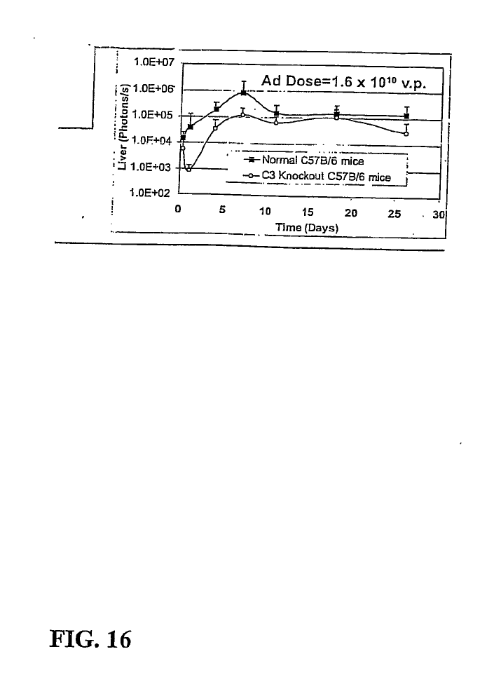

A new imaging method was developed to rapidly assess inflammatory response in vivo. Complement knockout (C3) C57B/6 mice showed from 10-30 fold lower luciferase expression in liver following intravenous injection of Ad5Luc1. The potential role of complement in liver clearance of Ad vectors was investigated. A role of complement in liver transduction by the Ad5Luc1 (normal Ad5 fiber structure, replication deficient) was found. As shown in FIGS. 15-16, the C3 knockout mice showed significantly lower levels of luciferase expression, and required longer exposure times to detect the liver luciferase expression. C3 is the primary effector molecule of complement activation. The C3 knockout mice strain were exactly matched to the C57B/6 control strain that was used. A sufficient number of animals were used in each group (n=4-5), and a second experiment showed a similar result using a higher i.v. dose of the Ad5Luc1 vector.

The Ad vector induces systemic and mucosal antibody responses. A replication incompetent Ad5 vector encoding lacZ was administered by intratracheal dosing in CD-1 mice or by conventional intranasal and intraperitoneal routes of injection. Kinetics of serum IgG, IgA, and IgM antibody responses to the Ad5 vector and to β-galactosidase (β-gal) were evaluated. Two or three adenoviral vector doses given by all 3 routes resulted in serum IgG titers in excess of 1:200,000, whereas serum IgM and IgA were moderately induced. Analysis of the predominant murine IgG subclass was determined to be IgG2a and IgG2b. To determine the localization of this antibody response, the ELISPOT assay was employed. Briefly, cells were isolated from the lung, the lower respiratory lymph nodes (LRLN), the nasal passages (NPL), and the spleen. For mucosally-administered Ad5, the highest IgA antibody-forming cell (AFC) response to Ad5 and β-gal was in the NPL and in the lung. Both the lung and the LRLN showed elevated numbers of IgG AFCs (4- to 12-fold greater than splenic IgG AFC responses) for both Ad5 and β-gal. It appears that the lung and associated lymphoid tissues were a main source of serum antibodies. Further analysis of serum antibodies showed that the intraperitoneal- and intratracheal-administered groups yielded the greatest neutralization titers to Ad5, showing reduced effectiveness of repetitive gene transfer was in part due to neutralizing antibodies in the circulation. Thus, repetitive intratracheal instillation stimulates a localized and systemic antibody response to this vector class.

Decreased efficacy of lacZ transduction correlated with increased immune reactivity to both Ad5 and β-gal. The reduction in lacZ expression with repeated viral administration was assessed to determine if it was due to an immune response against the β-gal or viral antigens. Efficiency and duration of transgene expression with antibody immune responses that are elicited by the viral vector are shown. Serum antibody levels were monitored in three groups of CD-1 mice that received one, two, or three doses of Ad virus at 2-week intervals. Serum IgG anti-Ad5 antibody levels peaked 30 days following the initial delivery with a titer of 1:16,000. Up to a 20-fold enhancement in serum IgG anti-Ad5 activity (titer between 1:200,000 and 1:300,000) was detected after two intratracheal instillations peaking between 15 and 30 days following the second adenoviral vector delivery. On the other hand, serum IgG anti-Ad5 levels increased only by a factor of two- to three-fold after 3 intratracheal doses. During the course of these intratracheal instillations, total serum IgG was enhanced by only three-fold. As anticipated, the adenoviral vector was immunogenic for CD-1 mice, and subsequent induction of an anti-adenoviral response reduced the efficiency of successive gene transfer in the lung. In addition, a serum IgG antibody response to β-gal was also detected in the same CD-1 mice. After subsequent Ad5-lacZ administrations, the titers to both Ad5 and β-gal increased in excess of 1:100,000. Thus, this evidence showed that CD-1 mice produced immune responses against both the adenoviral proteins and the transgene product (Dong J-Y Human Gene Therapy 7:319-331, 1996; Yuasa K, Gene Therapy 9:1576-1588, 2002; Thomas C E Human Gene Therapy 12:839-846, 2001; Moffatt S, Virology 272:159-167, 2000; Ruiz F E, Human Gene Therapy 12:751-761, 2001; van Ginkel F W, Hum Gene Ther 6:895-903, 1995; van Ginkel F W J. Immunol. 159:685-693, 1997).

Separate T Helper Cell subset responded to the Ad vector and expressed transgene. The induction of anti-viral immune responses can be divided into cell-mediated and antibody-mediated immune responses. The CD4⁺ T helper (Th) cells involved in these two pathways are of Th1-type for cell-mediated immunity (CMI), which likely contribute to clearance of Ad and other virally infected cells and CD4⁺ Th2-type which are involved in antibody-mediated immunity, contributing to immune exclusion and neutralization of viral vectors, for example, at mucosal surfaces. The role of these two major Th cell subsets for induction of specific immunity is in large part determined by the cytokines produced, where Th1 cells secrete IL-2, IFN-γ and LT-α, while Th2 cells secrete IL-4, IL-5, IL-6, IL-10 and IL-13. The induction of secretory IgA (S-IgA) antibodies at mucosal surfaces are often attributed to CD4⁺ T cells of the Th2-type and to their derived cytokines. For example, IL-4 plays an important role in the induction of Th2-mediated immune responses. IFN-γ and IL-12 are considered important immunoregulatory cytokines for the induction of a Th1-mediated CMI responses. In addition to regulating the types of Th cell responses, IL-4 and IFN-γ both have profound effects on the induced antibody responses. IFN-γ preferentially supports IgG2a while IL-4 provides help for IgG1 and IgE antibody responses in mice.

Pulmonary-associated CD4⁺ T cell subsets induced by Ad5-lacZ delivery to the murine respiratory tract were examined in order to assess their potential contribution to the immune responses which result from Ad-transgene delivery. These studies have important implications in the design of Ad and other viral vectors for gene therapy in which the vector is intended to circumvent the host's immune system and attenuate strong Th1-type responses (and subsequent inflammation). The immune events which occur in the lower respiratory tract following transfection of viral vectors can be described in terms of duration of transgene expression and induction of cytotoxic T lymphocyte (CTL) responses. Relatively less emphasis has been given in the past to understanding the contribution of CD4⁺ T cells in response to gene transfer vectors at mucosal sites. It has been found that both CD4⁺ and CD8⁺ T cells were found to migrate into the lung following sequential intratracheal Ad5-transgene administration. Isolated T lymphocytes from the lung and lower respiratory lymph nodes (LRLN) were more of Th2-type, and after cell sorting, the IL-4 producing T cells were largely CD4⁺, while IFN-γ activity was mainly associated with CD8⁺ T cells. Antibody responses to the Ad5 vector and to the expressed transgene β-galactosidase (β gal) revealed elevated bronchial and serum IgA and IgG antibodies with low neutralization titers. Analysis of IgG subclasses showed IgG1 and IgG2b with slightly lower IgG2a antibody responses to Ad vector and IgG2a- and IgG2b-responses to β gal. Cytokine analysis provided evidence for a mixed CD4⁺ Th1 and Th2-type response to Ad with preferential Th1-type responses to β gal (data not shown). Thus, Ad5-specific CD4⁺ T cells produced IL-4 with less IFN-γ, while β-gal-specific CD4⁺ T cells secreted IFN-β. This example provides direct evidence for the concomitant induction of Th2-type with lower Th1-type responses in both the systemic and mucosal immune compartments to the Ad5 vector and a Th1-dominant response to the transgene.

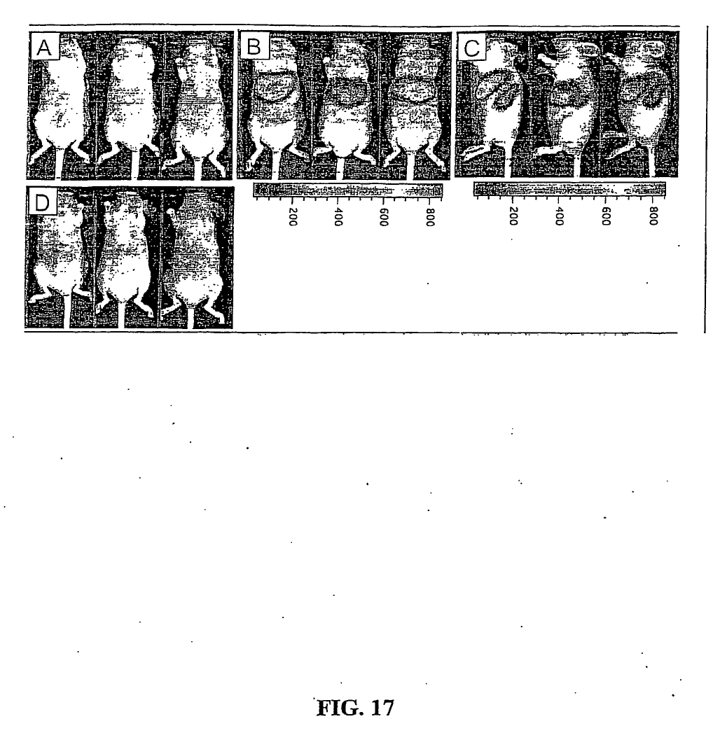

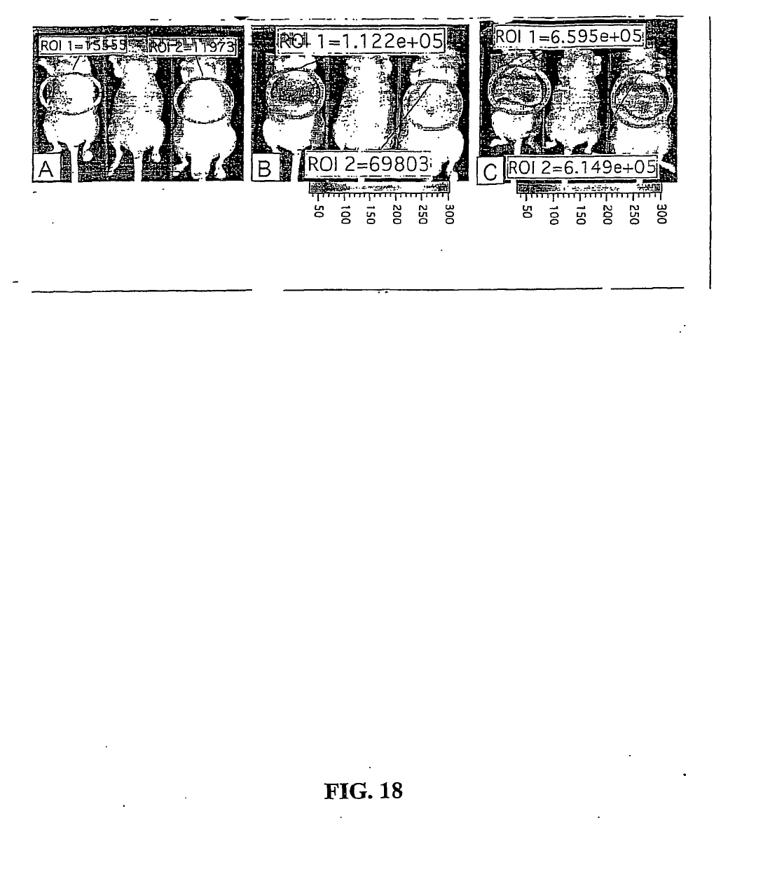

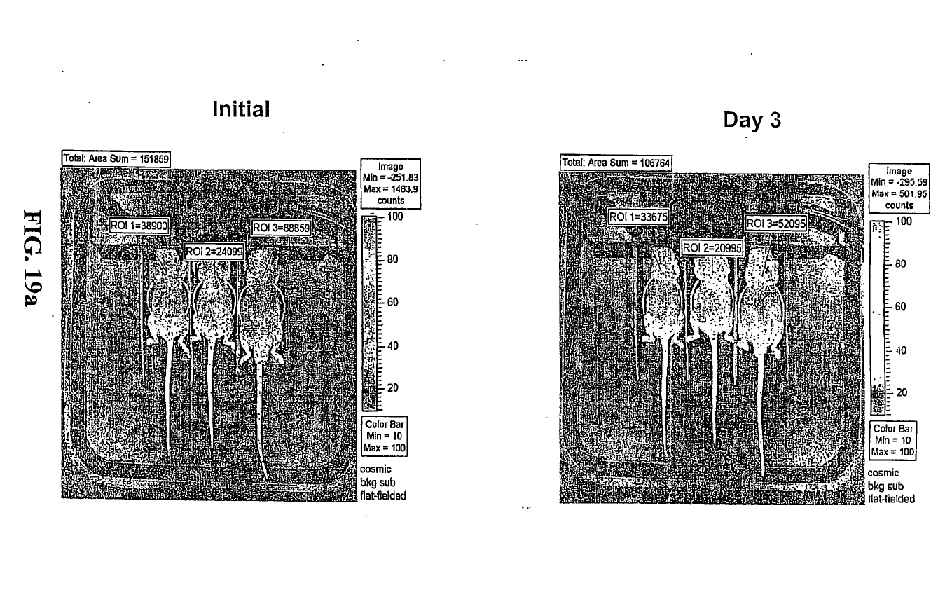

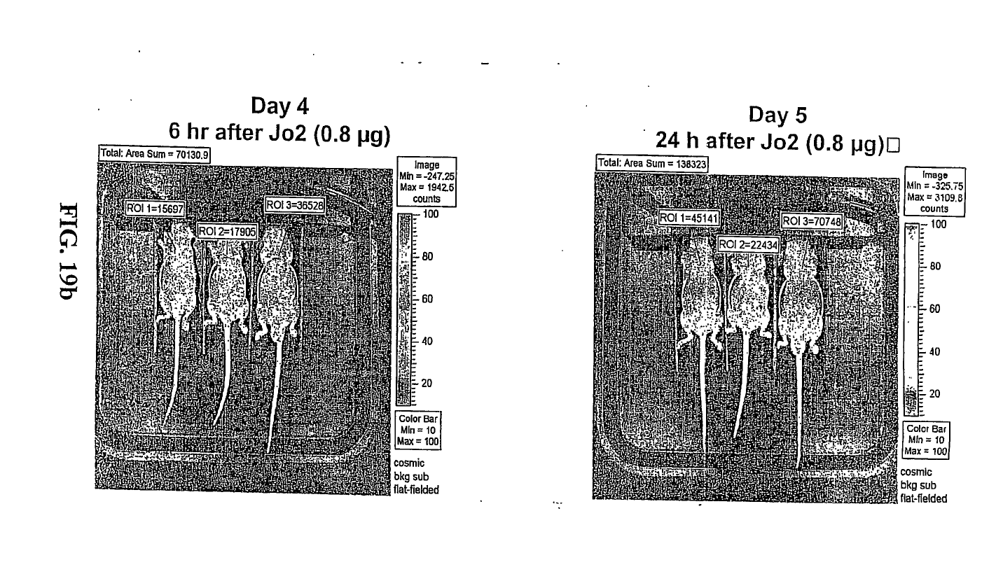

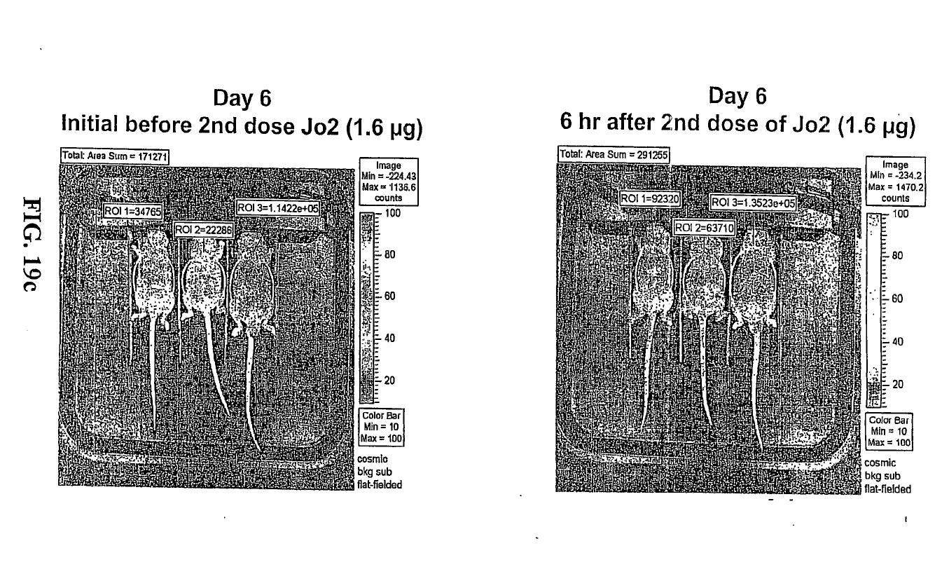

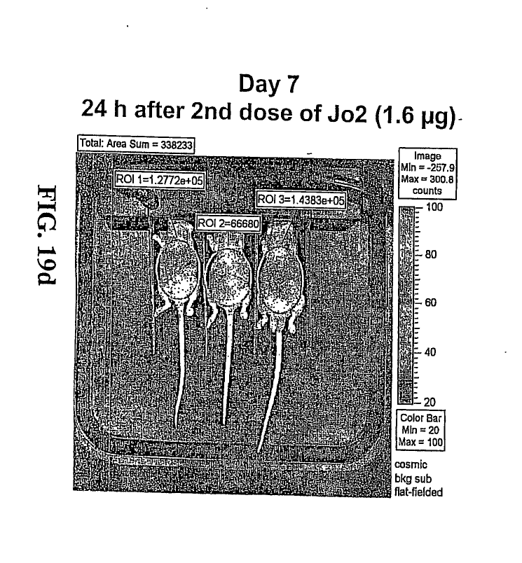

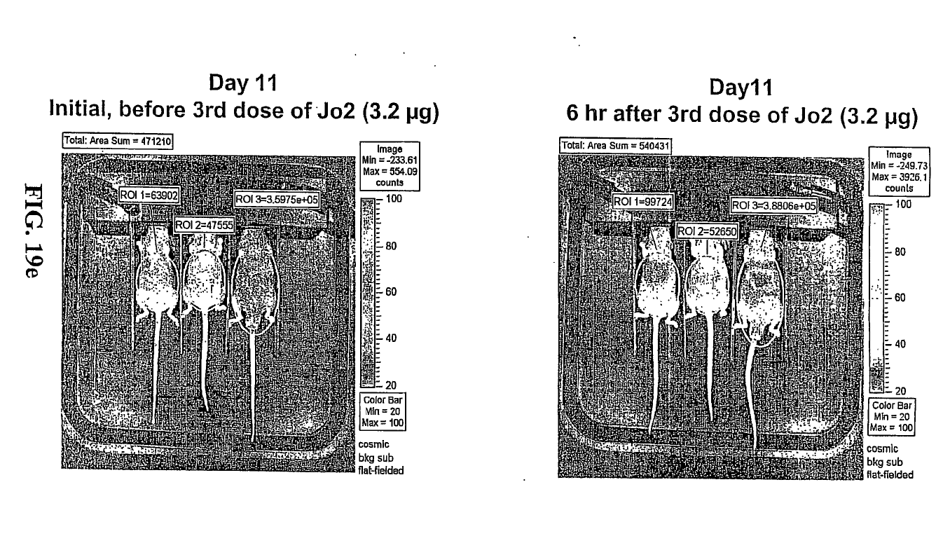

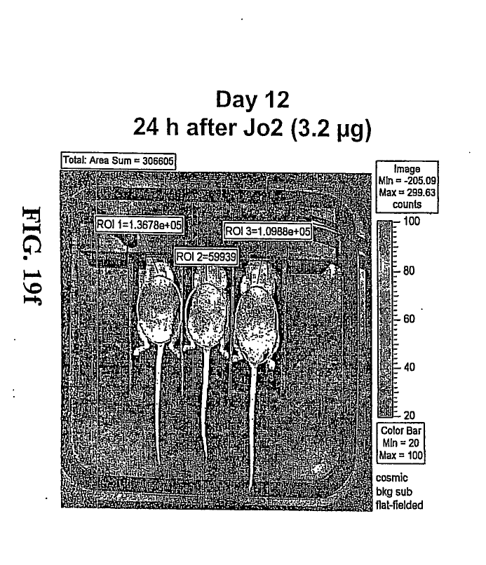

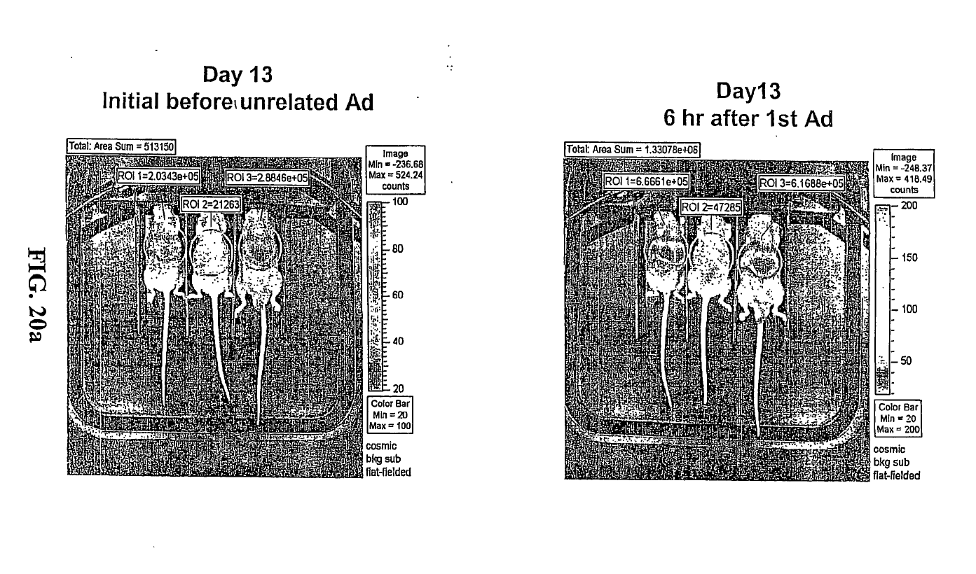

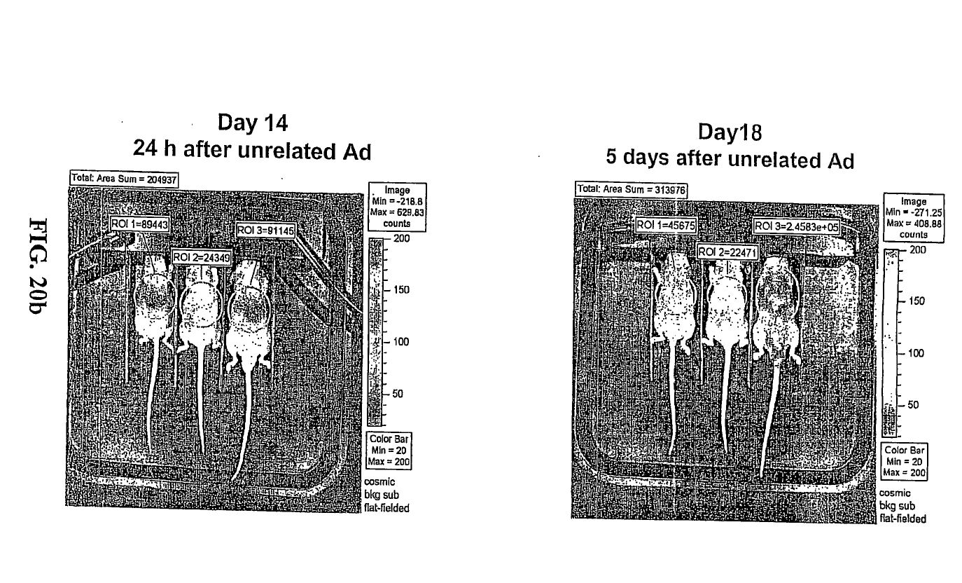

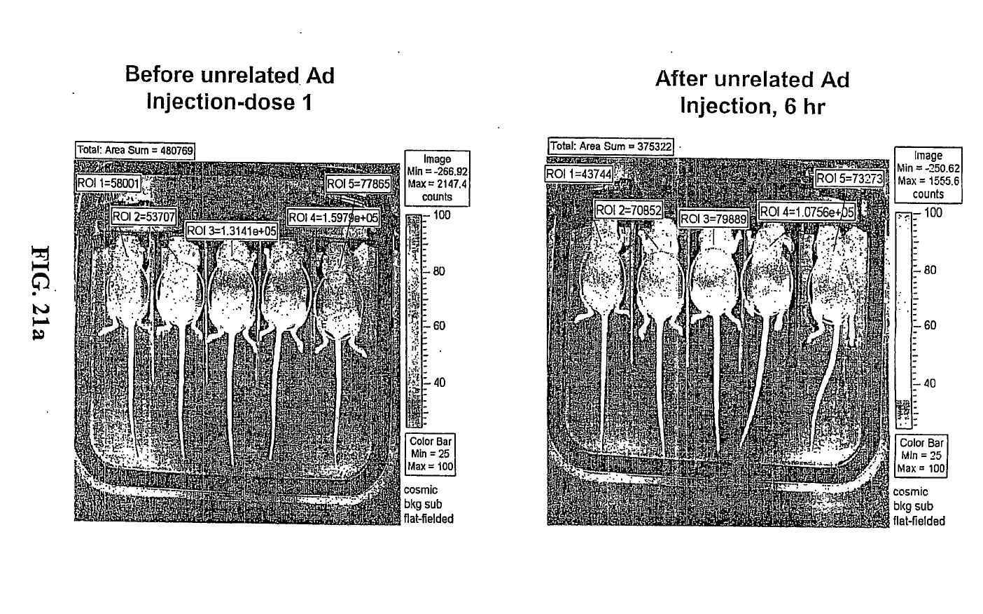

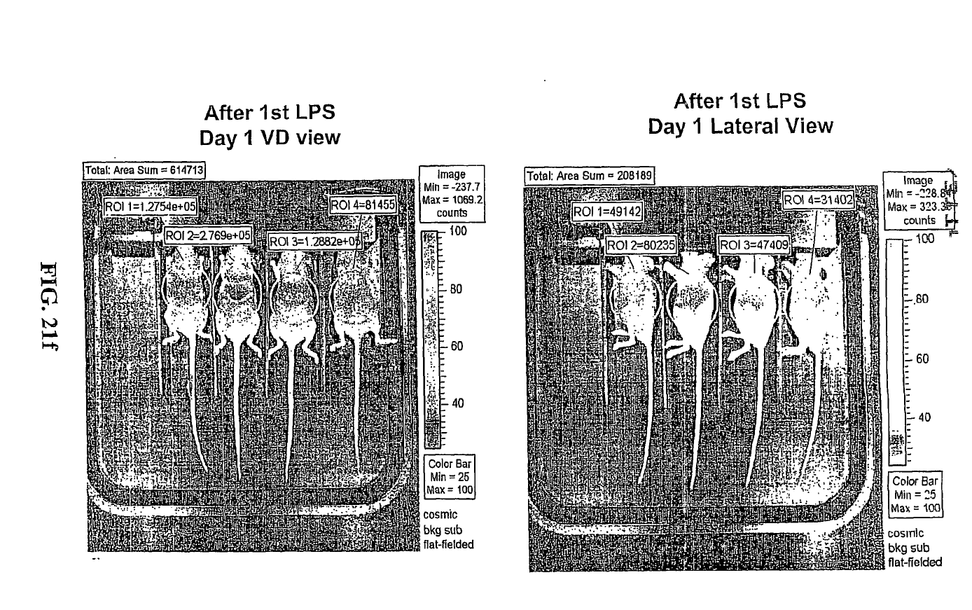

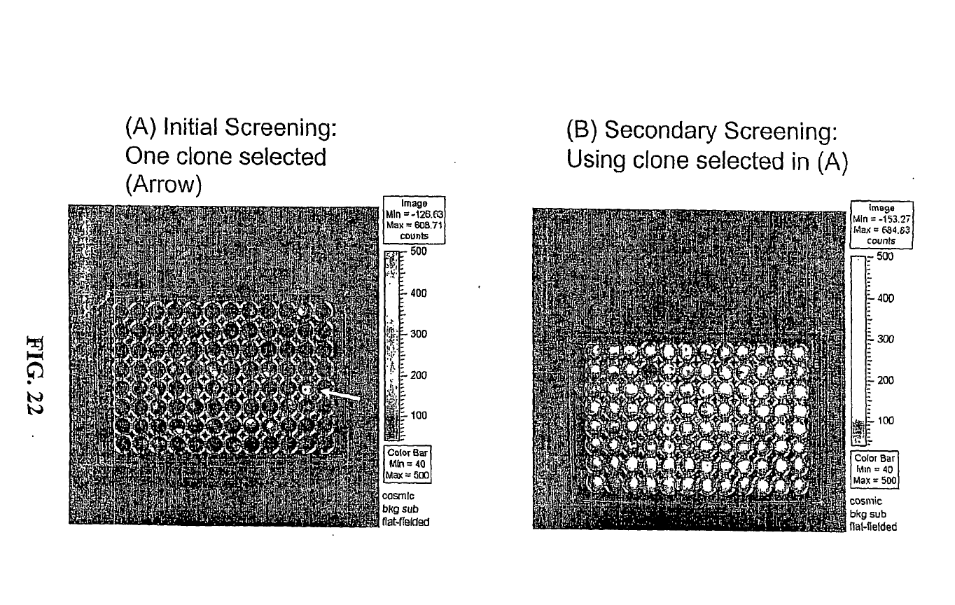

An imaging method was developed to assess inflammatory response in vivo. As shown in FIG. 17, a method has been developed to image liver inflammation. One experiment described here included five nude mice that were i.v. injected (2×10⁸ pfu) with a replication incompetent Ad vector encoding luciferase under control of the cox2L promoter. The promoter is not active in the normal liver, as shown with the 3 representative mice in FIG. 17A. All 4 images in FIG. 17 are on the same scale. Even at maximum sensitivity the liver luciferase expression could not be detected in FIG. 17A. However, at 4 hr after injection of a low dose of LPS (2 μg) the luciferase expression was induced by 12-fold over background signal (FIG. 17B-C). The expression was transient, and returned to absence of signal by 24 h after LPS injection. This system can also be used to non-invasively assess the inflammatory response to Ad vectors. Replacement of the LPS with a low dose of an irrelevant Ad vector in nude mice did not result in increased luciferase expression in liver. However, a significant response to the Ad vector was found with another condition. As shown in FIG. 22, 3 mice were injected first with Ad-cox2L-Luc, and 4 d later with a very low dose of an antibody that binds to FAS in liver, and induces hepatitis. The Jo2 dose was low, so only a mild, transient induction of liver luciferase expression was observed. However, when a subsequent low dose of an irrelevant Ad vector was i.v. injected, an inflammatory response in liver was observed, as detected by imaging the expression of luciferase after activation of cox2L. The capability to image inflammatory response in liver is important to assess Ad vectors that are delivered by i.v. routes. All images in FIG. 22 are on the same scale. The cox2L promoter is not active in the normal liver but is highly active under inflammatory conditions.

### 10. Example 10

### Ad Vector Development

Ad with fiber-fibritin (FF) chimeras are used as the platform for targeting, using genetic additions to the FF as the basis for targeting. The genetic FF additions include peptide sequences targeting E-selectin, the somatostatin receptor, and a rat tumor endothelium marker. The new Ad vectors are Tc-99m-labeled and tested in vitro for adherence to appropriate cells lines expressing the requisite receptors. The expression of the genetic reporters encoded in the Ad are imaged. Similarly, the Tc-99m-labeled Ad vectors (using methods previously described (Zinn K R Eur J Nucl Med 28(8):1027, 2001; Zinn K R, Eur J Nucl Med Mol Imaging 29:S107, 2002)) are injected in mice and the distribution determined by imaging. The same mice are imaged later for expression of the induced genetic reporters like luciferase.

A tumor-targeted Ad5 vector is prepared encoding luciferase with the FF-chimera fused to a peptide (DGDITWDQLWDLMK) (SEQ ID NO: 4) for targeting the E-selectin receptor (Zinn K R Arthritis Rheum 42:641-9, 1999). This sequence is known to have high affinity binding to mice, rat, and human E-selectin. Preliminary data validating the potential of FF platform to display ligands and achieve tumor-specific targeting was provided (both in vitro and in vivo), and showed that the liver luciferase expression was 100-fold reduced for FF-containing Ad, as compared with Ad with the normal fiber structure. In the second tumor-targeted Ad, the E-selectin binding peptide sequence is replaced with a peptide sequence for targeting the somatostatin receptor. Several peptide sequences are well established, where each shows high affinity binding across species, including to mouse, rat, and human somatostatin receptor (esp. subtypes 2 and 5) (Hoyer D, Trends Pharmacol Sci 16:86-8, 1995; Yamada Y., Proc Natl Acad Sci U S A 89:251-5, 1992; Feuerbach D Neuropharmacology 39:1451-62, 2000; Reubi J C, Eur J Nucl Med 27:273-82, 2000). The Tc-99m-labeled peptide (P2045) also binds to mouse and human somatostatin receptors (subtypes 2 and 5).

Two strategies are pursued in construction of the Ad vectors to reduce complement activation and inflammation. First, amino acid sequences known to bind negative regulators of human complement are included in construction of the Ad vector. One sequence that has potential is referred to as SCR 13-15. This sequence was recently shown to bind human complement regulator factor H on the pneumococcal surface (Duthy T G Infect Immun 70:5604-11, 2002), and thereby prevent complement activation. Potential sites of incorporation in the Ad include the Ad hexon, or pIX, a recently demonstrated site for genetic addition of proteins (Dmitriev I P, J. Virol 76:6893-9,2002). A linker site (poly GGGGS) (SEQ ID NO: 3) between the FF chimera and retargeting ligands is a second potential site (Krasnykh V J Virol 75:4176-83, 2001). Other potential sites would be available in the fiber, or the knob structure of the Ad vector. In this manner, a negative regulator of complement activation will bind the surfaces of the Ad vector, and thereby reduce complement activation. This strategy is used by certain microorganisms to by-pass innate immunity, and is supportive of the approach (Meri T Infect Immun 70:5185-92, 2002; Meri T, J Infect Dis 185:1786-93, 2002). The second strategy is to encode the negative regulators of complement directly within the genome of the Ad vector. In this manner, no binding of a blood factor is necessary, as the factor would be attached to the Ad vector when the Ad is assembled. The ideal candidate for initial evaluation of this approach is Crry protein, a complement inhibitor protein that has worked for this purpose in several model systems (Caragine T A Cancer Res 62:1110-5, 2002; Quigg R J J Immunol 155:1481-8, 1995). Expression of Crry on MCF7 cancer cells inhibited complement activation (C3) and increased the tumorigenicity of the MCF-7 cells in a rat breast cancer model.

Ad replication increases expression of the reporter signal. A replication competent Ad vector encoding luciferase (Ad5Luc3) is used, where replication is only possible in human tumors growing in the mice. The replication competent version of the Ad with FF chimera fused to CD40L is prepared.

### 11. Example 11

### Jo2 Antibody Increases Inflammation

Three experiments are included in this example. Experiments 1-2 were conducted with the same three mice. Experiment 3 used 5 different mice.

a) Methods

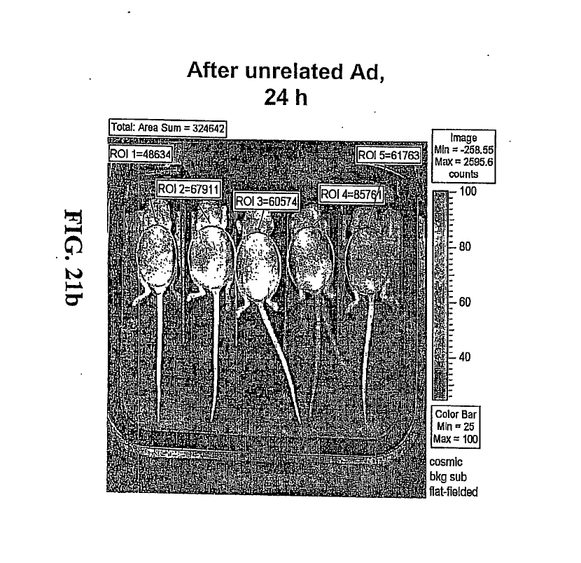

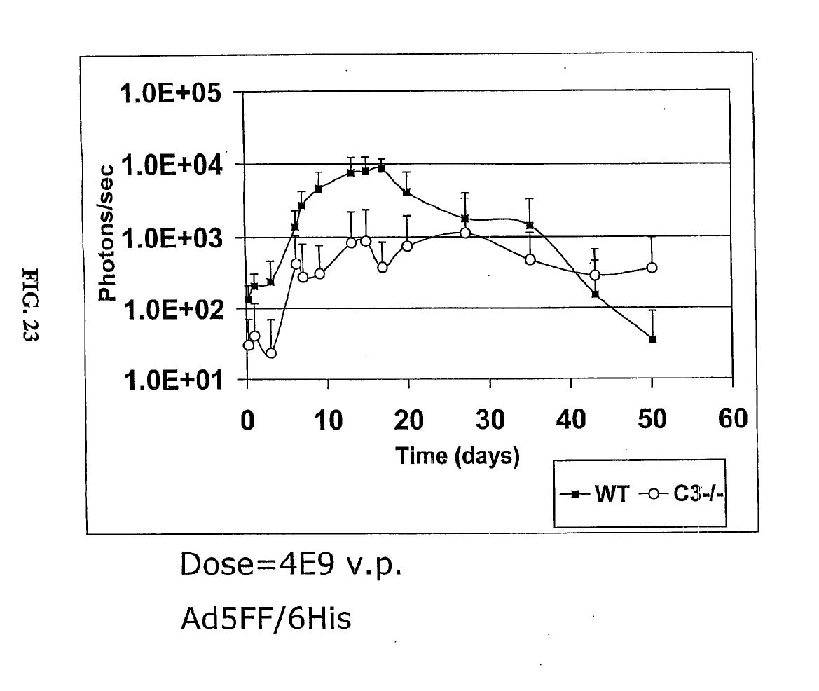

All mice were injected i.v. with a replication incompetent serotype 5 adenovirus (Ad) encoding luciferase (2×10⁸ pfu), under control of the cox2L promoter. Luciferase expression was detected by measuring light emission from the mice using a CCD camera (Xenogen IVIS system) at 15 min after injection of 2 mg of luciferin i.p. FIGS. 23-25 are overlays of mice images (black and white photographs) with pseudocolor images; the different colors represent the intensity of light emission from the mouse. The relative photons emitted in an area of the mouse was determined by region of interest analyses. For all mice, the luciferase expression in liver was extremely low, essentially undetectable by 3 days after dosing with Ad-cox2L-Luc (FIG. 19A).

b) Experiment 1

Increasing doses of Jo2 antibody (i.v. injected) to induce inflammation were evaluated. The lowest dose (0.8 μg) produced only mild increases in luciferase expression in ⅔ mice. A slightly greater response was noted for the next dose (1.6 μg), while the highest dose (3.2 μg) resulted in higher luciferase expression in liver by 6 h in ⅔ mice, and 24 h (FIG. 19). The luciferase expression in liver remained an additional 24 h. One mouse did not show luciferase expression in liver. The 3.2 μg Jo2 dose is not lethal, and is considered a mild stress to the liver.

c) Experiment 2

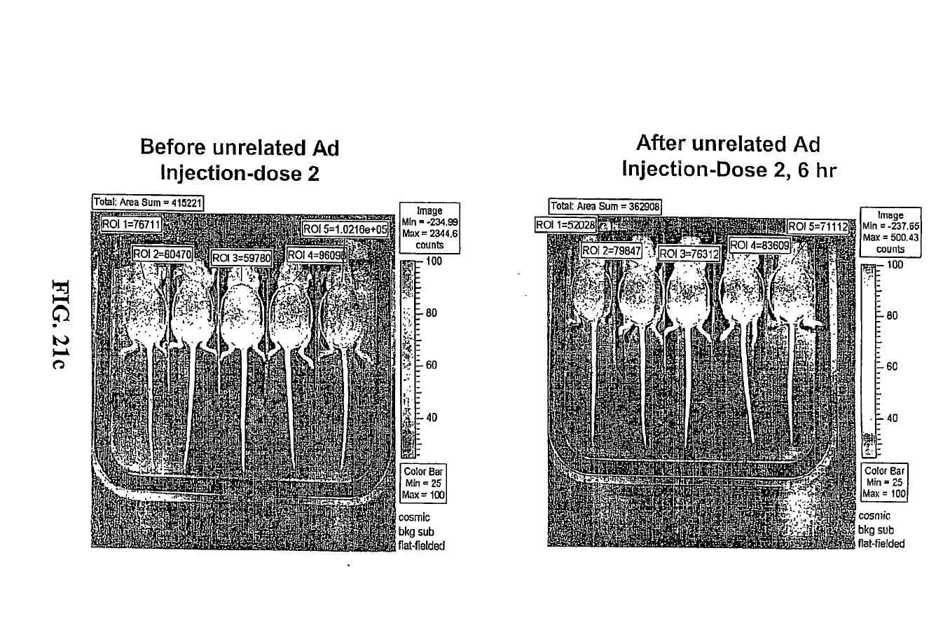

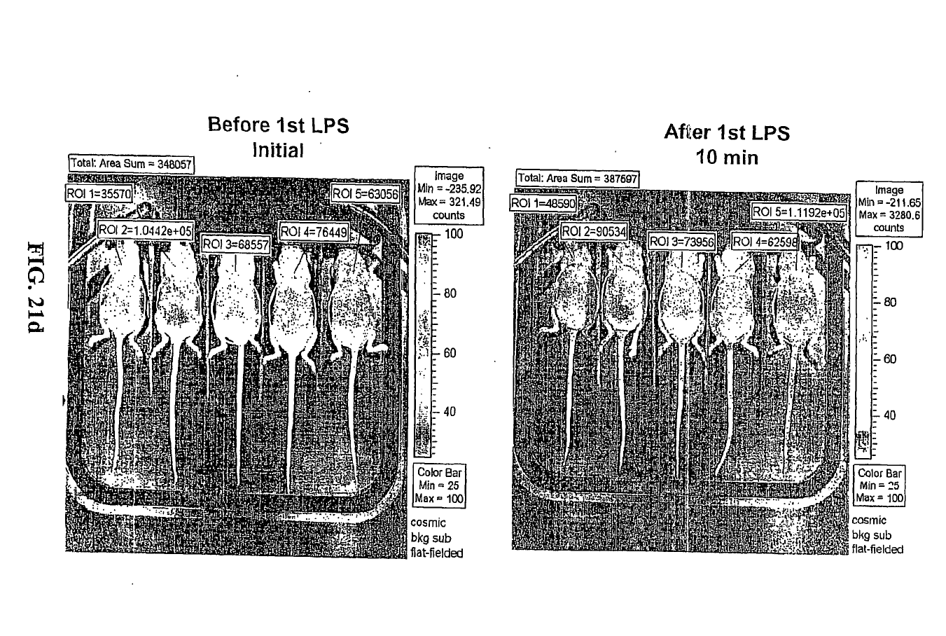

The same mice were injected with an unrelated Ad vector (3×10⁹ pfu) to simulate conditions where a gene therapy vector would be delivered to liver that was previously subjected to a mild inflammatory reaction (i.e. as simulated by Jo2). The unrelated Ad did induce luciferase expression in the liver in ⅔ mice. Of interest, persistent inflammation was detected in the male mouse in liver and testis even 5 days later (FIG. 20). In animals not treated previously with Jo2, the unrelated Ad dose did not increase luciferase expression in liver. These control studies are discussed under Experiment 3.

d) Experiment 3

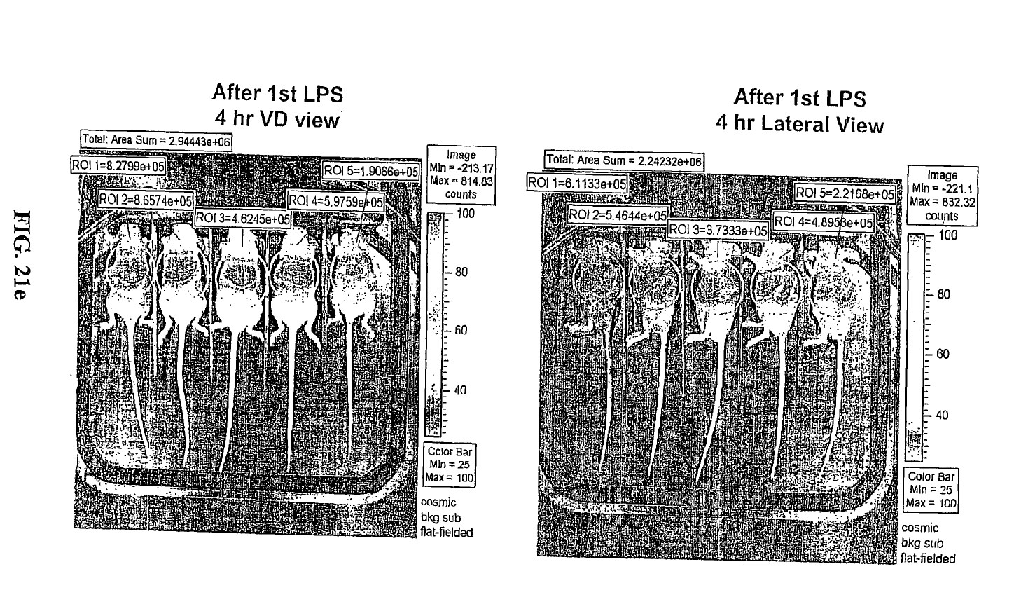

Mice were injected with the Ad-cox2L-Luc first, then with an unrelated Ad vector (same dose as Exp. 2). The unrelated Ad did not induce luciferase expression in liver, even after a second dose of unrelated Ad (FIG. 21). However, a low dose of LPS (2 μg) induced luciferase expression in liver and spleen by 4 h after LPS injection (FIGS. 21E and 21F). After 24 h the liver luciferase was reduced.

e) Conclusions

Ad-cox2L-Luc injected i.v. in mice can be used to monitor inflammatory status in the animals. This was demonstrated by the fact that low levels of Jo2 induced expression of luciferase in the liver that was detected by in vivo imaging. A mild liver injury with Jo2 resulted in changes in the liver that allowed subsequent injection of an unrelated Ad vector to produce luciferase activation, with persistence of luciferase signal (inflammation) in liver and other sites. Normal mice previously injected with Ad-cox2L-Luc, and then with the unrelated Ad twice, had no induction of luciferase expression in liver. However, low doses of LPS induced luciferase expression in liver of the same mice.

### 12. Example 12

### Method for Production of Luciferase-positive Cancer Cell Lines for Imaging

The method of this example includes two steps. First, a low number of cells (cancer cells or non-cancer cells) are infected with the adeno-associated virus (AAV) encoding luciferase. Next, the infected cells are diluted and transferred to 96-well plates, with the goal of obtaining 1-2 cells per well. After approximately 2 weeks the intact plate with live cells is imaged by the bioluminescence technique. As shown in the example presented in FIG. 22A, the imaging allows luciferase-positive cells to be identified. The positive clone is then subjected to another round of screening, as shown in FIG. 22B. In this example there were 95/96 wells that were positive, indicating the high percentage of luciferase-positive cells and efficiency of the technique. A positive clone was selected from the second round, and the process was repeated.

Numerous luciferase-positive cell lines can be established. These have applications for in vitro testing of cancer therapies as well as application for in vivo imaging. The in vivo tumor mass is related to the amount of light that is emitted from the tumors. If a therapy is working, then the tumor mass is less, and therefore less light emission. Also, the metastasis of the cancer can be detected by imaging. Luciferase-positive cells that are injected in animals can also be traced by this method. Advantages of this method include the stable integration of luciferase in the cell genome, and lack of requirement for a selectable marker.

The same method can be used to produce cell lines that are positive for GFP. Additional promoters can also be included, for example a promoter driving GFP that is controlled by CMV, while the luciferase is controlled by a second promoter that is active only in a certain cell type, or active only under certain conditions, e.g. activation of a biological process. Cell lines have been prepared that are luciferase positive by the method described herein, driven by CMV. Furthermore, these established cell lines have been used to evaluate cancer treatments.

### 13. Example 13

### Methods, Generally Applicable

a) Animal Models

(1) Nude Mice Xenograft Models

Subcutaneous xenograft tumor models are used, especially using the cell lines expressing the receptors that are targeted with the Ad vectors. Cell lines for implantation to produce xenograft tumors include A-427, SKOV3, and CRL-2116.

(2) Immune Competent Models

(a) C57BL/6 Tumor Models

C57BL/6 syngeneic tumor models are used. In one example, E-selectin knockout mice are used to validate E-selectin targeting of new Ad vectors to tumors. In another example, C57BL/6 C3 knockout mice are used to study the effect of complement on the targeting of the newly developed Ad vectors. MS1 (endothelial) and TC-1 (lung tumor) cell lines are used. Both allow evaluation of E-selectin and somatostatin receptor targeting with the new Ad vectors. In C57BL/6 mice, MS1 produces benign hemangiomas while TC-1 cells produce lung tumors at 100% frequency when 10⁴ cells are inoculated.

(b) Breast Tumor Models

Two models of breast neoplasia are included because breast cancers are accessible due to their surface locations. The tumors can be easily injected with Ad. Bioluminescence and fluorescence imaging applications can be used for detection of low signal. The CRL-2116 cell line is a breast tumor line that produces tumors in BALB/C mice. These cells are implanted s.c. to produce tumors. Expression of reporter genes in these tumors was imaged following s.c. injection of the tumors with Ad.

(c) Rat Mammary Cancer Model

The N-nitroso-N-methylurea (MNU)-induced mammary cancer model in rats is also be used. Sprague Dawley rats already injected with MNU (50 mg/kg body weight) are used. Treatment of the female rats at 50 days of age results in palpable tumors beginning at 35 days after carcinogen treatment. By 100 days after treatment there is an 80-90% incidence. As shown in FIG. 13-14, these tumors are somatostatin receptor positive, and show expression of the tumor endothelial-specific marker.

(3) Endpoints

The animals are imaged repeatedly over time (weeks to months) to measure reporter gene expression; certain experiments also measure immune response to the vector. Animals that are “cured” by therapy are maintained for 120 days to insure that tumors do not develop.

b) Specific Methods

(1) Methods Related to Ad Vectors

(a) Generation of Replication-defective Ad

The appropriate promoter is cloned into an Ad shuttle vector upstream of the gene of interest, for example the hSSTR2 gene, or modified hSSTr2 gene. Viral genomes are generated by homologous recombination using the plasmid pAdEasyl, which contains the majority of the Ad genome except for deletion of the early region 1 and 3 (E1 and E3) genes. Viruses are generated by transfection of the linearized Ad genome plasmid into the E1 transcomplementing cell line, 293. Viral DNA is isolated and assessed by restriction analysis and partial sequencing. Viral stocks are generated in 293 cells, purified by centrifugation through two cesium chloride gradients, then titered by determining OD260 and by plaque titer on 293 cells. Bicistronic vectors are constructed in which the hSSTr2 reporter is combined with the therapy gene in the same Ad.

(b) Generation of Ad with Fiber-fibritin (FF) Chimera

This Ad construct was designed to expand the repertoire of the targeting ligands and also to address the issues of the unfavorable biodistribution of Ad vectors in vivo. The gene encoding the chimera is incorporated into the genome of a luciferase-expressing Ad vector, which is propagated according to a two-step scheme developed by Von Seggern (Von Seggern D J, J Virol 74:354-62, 2000). First, the virus is rescued in 211B cells expressing wild type Ad5 fiber. At this point the virions contain the FF chimeras and wild type fiber, which allows for subsequent infection of regular 293 cells. The virions released from 293 cells exclusively incorporate FF chimeras—no wild type fiber is present. The resultant Ad virions are purified on CsCl gradients at high titer (equivalent to Ad with normal fiber). The presence of the FF in the virions is confirmed by SDS-PAGE and Western blot analyses.

Using Ad vector(s) incorporating FF chimeras in the proposed work is based on two important considerations related to the biodistribution of Ad-based vectors. First, it has become apparent that due to its interaction with broadly expressed CAR, the knob domain of the fiber protein contributes heavily to unfavorable distribution of Ad vectors in vivo. Additionally, it has been shown that the KKTK (SEQ ID NO: 1) motif within the Ad5 fiber shaft domain further complicates the issue of the vector's tropism by mediating its binding to heparin sulfate proteoglycans expressed in the liver. In this regard, FF-containing Ad vectors are unique in that they do not contain either the fiber knob or the KKTK (SEQ ID NO: 1) tetrapeptide in the shaft and therefore allow for bypassing the natural mechanism of the vector's sequestration in vivo.

(c) “Double-ablated” Ad

The double-ablated Ad vectors that lack CAR and integrin binding are used. The vectors are Tc-99m-labelled, and in vivo kinetics of clearance are determined, and image reporter gene expression is carried out in the same mice. These results are compared with FF-containing Ad vectors.

(d) Generation of Replication Competent Ad Vectors

Replication competent Ad vectors are generated as described above for replication deficient vectors, with the difference being that transgene-encoding cassettes are incorporated in place of the E3 region of the Ad genome.

(2) Human-origin Cells Lines

A-427, SKVO3, HUVEC, and 293 cell lines are used. HUVEC cells are easily induced to express E-selectin with IL1β, as previously described (Zinn K R Arthritis Rheum 42:641-9, 1999).

(3) Mouse-origin Cell Lines

A mouse origin breast tumor cell line (CRL-2116) is used: it produces tumors in immune competent BALB/C mice. Also, the MS1 (endothelial) and TC-1 (lung tumor) cell lines are purchased from the ATCC. In C57BL/6 mice, MS1 produces benign hemangiomas (high levels of E-selectin) while TC-1 cells produce lung tumors at 100% frequency when 10⁴ cells are inoculated.

(4) Radiolabeling

Proteins and peptides are easily modified with succinimidyl 6-hydrazinonicotinate (HYNIC, 3:1 molar ratio, 3 h). Dialysis overnight against phosphate buffered saline removes unreacted HYNIC. The HYNIC-modified constructs are radiolabeled with Tc-99m using tricine as the transfer ligand and purified from non-bound Tc-99m by G-25 Sephadex size exclusion chromatography. Care is taken (Scatchard Analyses, plate imaging assays) to validate that attachment of the radioisotope does not change binding kinetics. With respect to hSSTr2-avid P2045, the peptide contains an N₃S system to allow radiolabeling with either Tc-99m or Re-188. Precise conditions for radiolabeling with at least 4 hr of high stability have been established for each radionuclide. For Ad vector labeling, a preformed Tc(l) chelate is prepared per established methods (Waibel R, Nat Biotechnol 17:897-901, 1999). The preformed chelate to Tc-99m label proteins with a 6-His tag can also be used, thereby providing another tool for radiolabeling peptides and proteins. I-125 and I-131 labeled FIAU are produced as previously accomplished (Zinn K R Radiology 223:417-25, 2002).

(5) Conjugation of Cy3 and Cy5 to Proteins

Kits from Amersham are used that contain succinimidyl derivatives of the dyes. Numerous peptides/proteins have been labeled using the protocols developed for HYNIC attachment. These conjugates are imaged by confocal microscopy, or used in flow cytometry.

c) Imaging

Imaging determines the amount of Tc-99m-Ad that leaks from a directly injected s.c. tumor, and to what sites the Tc-99m-Ad becomes localized. Imaging assesses the fraction of intravenously injected Tc-99m-labeled Ad that becomes bound in the tumor. The Tc-99m-Ad targets the tumor-specific receptors. The same animals are repeatedly imaged after 24-48 hours to determine the level and persistence of reporter gene expression. Immunohistochemistry determines the distribution and the cell types of the expressed reporters within the tumor. Co-expression of hSSTr2 and TK within the same regions of Ad-hSSTr2-TK infected tumors has been validated. Simultaneous in vivo imaging for detection of the hSSTr2 and TK takes place. 3 different fluorophores can simultaneously be detected by confocal microscopy, i.e. FITC/GFP channel 1, Cy3 channel 2, and Cy5.5 channel 3.

(1) Gamma Camera Imaging

Three gamma cameras for planar imaging are used. In addition, a SPECT/CT camera (GammaMedica, Inc.) for 3-dimensional rodent imaging can also be used. The data presented in FIG. 6 showed the capability to fuse SPECT images with anatomical CT images. The software provided with this system reconstructs the images, and fuses them automatically. The images can be presented a 1-mm slices, or as volume renderings. Tumor regions are evaluated by manual region of interest (ROI) analyses. The total number of voxels within the ROI is used to determine the Ad-infected volume of the tumor, since each voxel corresponded to 1 mm³.

The rat model system allows for the evaluation of both the location of Ad delivery within tumor, and the location of expressed mutant hSSTr2 reporter that was delivered with the Ad. The tumors express somatostatin receptor, and therefore the tumor location can be imaged with In-111-octreotide. Simultaneously, the Tc-99m-labeled Ad will be imaged to determine the location within the tumor. The gamma camera can detect Tc-99m and In-111 simultaneously. After decay of the initial Tc-99m dose (2 days), the expressed mutant hSSTr2 reporter gene is imaged with a second Tc-99m-ligand that is specific for the mutant hSSTr2. In this manner, the 3-dimensional location of Ad and transgene expression is determined in the tumor.

(2) Bioluminescence Imaging

A Xenogen IVIS-100 system for bioluminescence imaging with upgraded capability for fluorescence imaging can be used.

(3) Fluorescent Stereomicroscopic Imaging

Techniques that known in the art to image live mice can be used (Chaudhuri T R, Gynec Oncol 80:330, 2001; Chaudhuri T R Cancer Biother Radiopharm 17:205-212, 2002.) The ORCA-ER CCD camera that is part of this system has sensitivity to detect light out to 1000 nm (near infrared), and the light source is capable of excitation in this range. Multiple objectives allow for a wide range of imaging, from the whole animal to monitoring individual cells. Appropriate filter modules in a turret allow quick change for different fluorophores.

(4) Quantization of Light-based Imaging Signal

Calibration standards are imaged in established positions to insure a constant detection signal under each condition. For fluorescence, fluorophore-loaded beads are used that are commercially available. Consistency of the illumination source is evaluated. For bioluminescence, a long-lived radioisotope with scintillation fluid is used. For both light-based methods the intensity of signal per pixel is determined using region of interest analyses.

Ad vectors are developed that specifically target tumor-specific receptors, and which reduce immune activation. The Ad vectors will be imaged following i.v. injection, to determine in vivo targeting. At later times, the expression of genetic reporters (luciferase, hSSTr2) is measured in the same animals. These data are compared to studies of immune activation.

d) Antibody Determinations

In general, vector-specific antibody titers and total Ig levels can be determined by ELISA assays as previously described and modified for specific vector subtypes (van Ginkel F W, J. Immunol. 163:1951-1957, 1999; Pascual D W Int. Immunol. 3:1223-1229,1991). Gene transfer vectors (for example, 1×108 Ad5 particles/well or specific transgenes such as LacZ at 0.2 μg/well; Sigma) are coated onto Nunc Maxisorp Immunoplates II microtiter plates (Fisher Scientific, Atlanta, Ga.) overnight at 4° C. in 100 μl of sterile PBS, pH 7.2. Varying dilutions of mouse sera are diluted in ELISA buffer (PBS, 0.5% BSA, 0.05% Tween 20), and incubated overnight at 4° C. Reactivity to vector or transgene is determined with horseradish peroxidase conjugates of detecting antibodies (1 μg/ml): goat anti-mouse IgG, IgM, IgA antibodies [Southern Biotechnology Associates (SBA), Birmingham, Alabama], and monoclonal antibodies specific for IgG1, IgG2a, IgG2b, and IgG3 (PharMingen). Following a 1.0 hr incubation at 37° C. and several washing steps, specific reactivity is determined by the addition of horseradish peroxidase enzyme substrate, 100 μl/well of 0.1 mg/ml of 2.2′-azino-bis(3-ethylbenzthiazoline 6-sulfonic acid) diammonium (Sigma) in 0.1 M citrate buffer pH 4.5, and 0.01% H₂O₂, and absorbance read at 415 nm on a Kinetics Reader model EL312 (Biotek Instruments, Winooski, V T). Endpoint titers are expressed as the reciprocal dilution of the last sample dilution giving an absorbance≧0.1 OD units above the OD₄₁₅ of negative controls after a 30 min incubation.

e) Lymphoid Cell Isolation

Lymphocytes are isolated from spleen, lung, lower respiratory lymph nodes (LRNL), nasal passages (NP), Peyer's patches (PP) and lamina propria (LP). Single mononuclear cell suspensions are obtained from each (except lung, PP and LP) by mechanically disrupting them followed by centrifugation over a Ficoll-Hypaque density gradient (Lymphocyte M, Accurate Chemicals, Westbury, N.Y.) and collection of the interface containing lymphocytes. Isolation of mononuclear cells from other tissues are performed as previously described (van Ginkel F W Hum Gene Ther 6:895-903, 1995; van Ginkel F W, J. Immunol. 159:685-693, 1997; Simecka J W, Infect. Immun. 59:3715-3721, 1991; Simecka J W, Reg. Immunol. 4:18-24, 1992; Nguyen H H J. Infect Dis. 183:368-376, 2001; Jones H P, J. Immunol. 167:4518-4526, 2001). For preparation of PP and LP cells, we routinely use enzymatic dissociation with the enzyme dispase® (1.5 mg/ml). These methods are as previously described. To assess mononuclear cell purity isolated from the tissues, approximately 1×10⁴ cells are applied to bovine serum albumin (BSA)-coated slides and concentrated by centrifugation using a Shandon Cytospin 3 (Astmoor, England); the cells are stained with a combination of eosin and thiazine (Hemocolor, EM Diagnostic Systems, Gibbstown, N.J.), to determine the percentage of lymphocytes in these cell fractions. In these experiments, ˜60-65% lymphocytes are routinely obtained. Greater than 98% viability is usually noted for lymphocytes isolated from each tissue as determined by trypan blue exclusion.

f) ELISPOT Assay

The enzyme-linked immunospot (ELISPOT) is one of the most sensitive tools currently available to analyze B-cell antibody (Ab) and T-cell cytokine responses as well as other secreted molecules. Further, the secretion of Abs/immunoglobulin isotypes/subclasses or cytokines can be assessed by this technique at the single cell level. An antibody ELISPOT assay is used exactly as previously described (van Ginkel F W, J. Immunol. 163:1951-1957, 1999; Pascual D W Int. Immunol. 3:1223-1229, 1991). Nitrocellulose-based microtiter plates (Millititer, Millipore Corp., Bedford, Mass.) are coated with a vector of interest (for example as Ad virus at 1×10⁸/particles/well) or with 2.0 mg/ml of transgene (for example, 0.2 μg/well of β-gal) overnight at 4° C. The plates are blocked with complete medium containing RPMI 1640 (<0.1 ng/ml endotoxin; Whittaker BioProducts, Walkersville, Md.) supplemented with 0.2 mM L-glutamine, 0.1 mM nonessential amino acids, 0.1 mM sodium pyruvate, 100 U/ml penicillin, 100 μg/ml streptomycin, 10 mM HEPES (GIBCO, Grand Island, N.Y.), and low endotoxin 10% FCS (Hyclone, Logan, Utah.). A total of 0.1 ml of cells from each organ at a concentration of 5×10⁶ and 5×10⁵ lymphocytes/ml, with the exception of the NP which are normally at a concentration of 1-2×105 lymphocytes/ml are added to the ELISPOT microtiter wells. The cells are incubated for 12 hr at 37° C., 5% CO₂ after which the cells are removed from the plates with PBS (3×) and PBS-0.1% Tween (3×). For detection, goat anti-mouse-IgM, -IgA, and -IgG (SBA) conjugated to horseradish peroxidase in PBS, 0.5% BSA, 0.1% Tween 20 will be added to microtiter plates at 1 μg/ml (100 μl/well) and incubated overnight at 4° C.

Antibody or cytokine-secreting cells are visualized by addition of the peroxidase substrate, 3-amino--ethylcarbazole. The color reaction is stopped with H₂O after 1 hr of incubation at room temperature.

g) T-cell Subset Analysis

T cell analyses are performed to determine the nature of T cell response induced by viral vectors or transgene. Initial experiments in many cases may involve isolating lymphocytes following systemic administration of viral vectors and assessment of cytokine production by cytokine-specific ELISPOT. This is followed 10 by a more detailed analysis of CD4⁺ and CD8⁺ T cells separated by flow cytometry (using a UAB core facility) to assess cytokine production by a quantitative RT-PCR. It is of importance to determine whether the microenvironment of tissues following viral gene transfer is supportive of a Th2-dominated immune response for these vector systems. Moreover, in order to determine the relative stimulation of T cells by the vector versus the expressed transgene protein, in vitro stimulation of T lymphocytes from NALT, lungs, LRLN and spleen is also performed.

h) Quantitative RT-PCR for Cytokine mRNA Analysis

The quantitative RT-PCR method uses a recombinant DNA (rcDNA) internal standard specific for murine Th1 (IL-2 and IFN-γ) and Th2 (IL-4, IL-5, IL-6, and IL-10) cytokines. This connected rcDNA internal standard for these cytokines was generated and consists of recombinant PCR primer sequence, a 5′ cytokine-specific primer, a poly d(T)₁₆, a 3′ cytokine-specific primer, inserted into a pGEM-T plasmid containing the T7 promoter. A dedicated LightCycler® (Roche, Inc.) can be used, which quantitates all cytokine-specific and other mRNAs by real time RT-PCR.

i) Complement Neutralization and Deposition Tests

Either purified complement or serum (human or rodent, tested to be free of anti-Ad antibody) is incubated with the new Ad vectors, and infectivity is determined. For deposition tests, plates are coated with Ad vector, incubated with serum, then ELISA assays conducted for activated complement factors on the Ad.

j) W-188/Re-188 Generator

This system allows for cost-effective elution of Re-188 (17-hr half-life) as needed over the life of the generator (5-6 months). The Re-188 is no-carrier-added, and in concentration form. The necessary additives to maintain stability of Re-188-labeled peptides have been determined, protecting from radiolysis effects. A further advantage of Re-188 is the fact that it can be imaged; its 155 keV gamma-ray emission is similar in energy to that of Tc-99m (140 keV).

k) Therapy Studies

The Ad vectors and radiolabeled peptides are shared from a common stock. Therapy studies include injection of 5-fluorocytosine 2-4× per week, with radiolabeled peptides 2×/week for 6 doses (dose maximum=0.5 mCi). Tumors are measured twice weekly. The studies incorporate bioluminescence imaging of luciferase-positive tumors to assess response.

### 14. Example 14

### Tc-99m 3-dimensional Imaging in Lung

The precise location of radioactivity can be determined using the combined approach of SPECT and CT. An antibody targeting lung endothelium is used.

a) Methods

(1) Tc-99m Radiolabeling

Chemicals were from Fisher (Pittsburgh, Pa., USA) unless otherwise noted. The 99mTc (pertechnetate) was supplied by Central Pharmacy (Birmingham, Ala., USA). Tx3.833 antibody (0.400 mg) was modified with succinimidyl 6-hydrazinonicotinate (HYNIC:Antibody molar ratio, Exp. 1=16, Exp. 2=8, 3 h) and dialyzed overnight against PBS (pH 7.4), as previously described (Zinn K R, Nucl Med Biol 27:407-14, 2000; Abrams M J, J Nucl Med 31:2022-8, 1990; Rogers B E, Gene Ther 10:105-114, 2003.). The HYNIC-modified antibody was radiolabeled with 99mTc using tricine as the transfer ligand and purified from non-bound 99mTc by G-25 Sephadex size exclusion chromatography (Larsen S K Bioconj. Chem 6:635-8, 1995). Protein concentrations of the collected fractions were determined by the method of Lowry (J Biol. Chem 193:635-8, 1951). Protein bound 99mTc was greater than 99%, as determined by thin layer chromatography. The 99mTc-labeled, HYNIC-modified Tx3.833 (99mTc-HYNIC-Tx3.833) is designated as 99mTc-Tx. The specific activity of the 99mTc-Tx was 371 and 101 MBq/nmole for experiments 1 and 2, respectively.

(2) Animal Experiments

All injections were i.v. via the tail vein while rats were maintained under Enflurane gas anesthesia. Experiment 1 was conducted with 6 rats that averaged 182±10 g. ^{99m}Tc-Tx doses were 6.7 μg for rats 1-4, (for rat 4, the ^{99m}Tc-Tx was mixed with 0.200 mg unlabeled Tx3.833), and 1.3 μg for rats 5-6. After imaging procedures, the rats were terminated at 1.7 hr. Experiment 2 used 3 rats with an average weight of 209±5 g. The rats were injected with 28±1 μg of ^{99m}Tc-Tx and terminated after 23 hr.

(3) Imaging

Syringes were measured before and after injection using an Atomlab 100 dose calibrator (Biodex Medical Systems, Shirley, N.Y., USA). During imaging, the rats were maintained with Enflurane anesthesia. For dynamic and static protocols, the rats were positioned with their ventral surface facing the collimator. Dynamic imaging (300 frames, 10sec/frame) was accomplished with an Anger 420/550 mobile radioisotope gamma camera (Technicare, Solon, Ohio, USA) equipped with a pinhole collimator. Rats were imaged by static planar techniques, with at least 50,000 total counts per image collected. Dynamic images were processed with a modified version of NIH Image (Nuc Med Image, Mark D. Wittry, St. Louis University, St. Louis, Mo., USA) using standard manual region of interest (ROI) analyses. The ROI was drawn to include only the lung area.

Dual modality SPECT and CT images were collected using the A-SPECT system (GammaMedica, Inc., Northridge, Calif., USA). For the SPECT series, a total of 56 individual images (30 sec/image) were collected using a 1 mm pinhole collimator. Each CT series included 256 views; one series was collected without contrast, while a second was collected at 20 sec after i.v. injection of 0.5 mL iohexol (Omnipaque™, Amersham Health, Princeton, N.J., USA). The reconstructed SPECT and CT images were fused to allow precise localization of ^{99m}Tc-Tx. Images presented with SPECT-CT overlays represent 1.0 mm slices.

(4) Tissue Collection and Data Reduction

After imaging, the tissues were collected, weighed, and counted in a Minaxiγ Auto-Gamma 5000 series gamma counter (Packard, Downers Grove, Ill., USA). After the tissues were removed, the remaining carcass was subdivided into vials for counting. Each entire rat was measured in the gamma counter. The raw count rate data from the gamma counter were decay corrected to the injection time. Radioactivity in the tissues were normalized to the tissue weight and dose, and expressed as % of injected dose per gram of tissue (% ID/g). The % ID was estimated for the total blood using a total blood equal to 7% of body weight. For this estimation, the % ID/g for blood was multiplied by the total mass (g) for the blood.

b) Results

(1) Imaging Studies

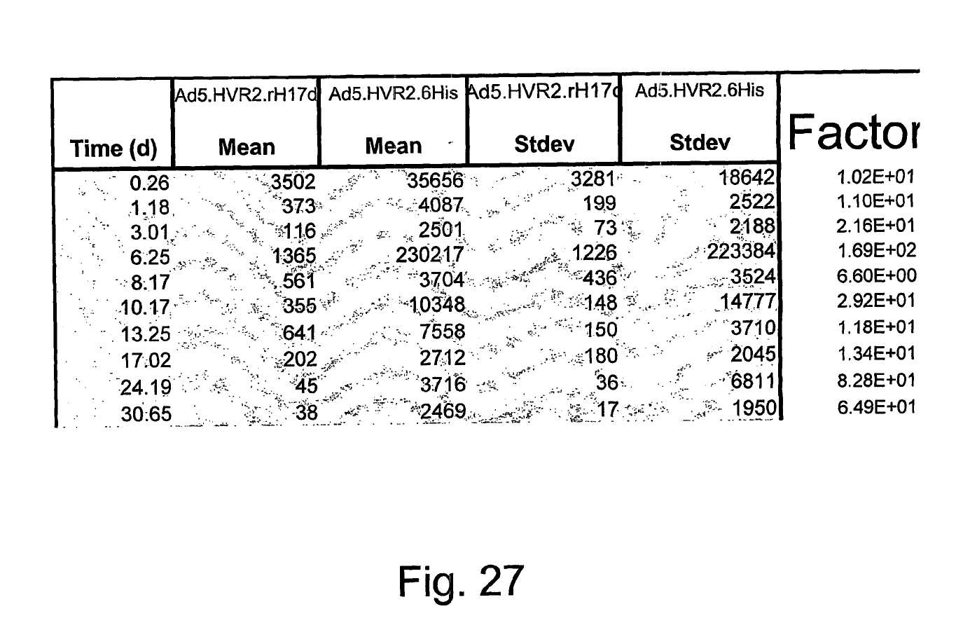

ROI analyses results of the dynamic imaging studies with ^{99m}Tc-Tx are presented in FIG. 27 for two representative rats. Lung binding of ^{99m}Tc-Tx was rapid, and reached equilibrium by 60 sec after injection. These studies also demonstrated that the lung binding was specific, as there was a significant reduction when ^{99m}Tc-Tx was diluted with unlabeled antibody. Further confirmation that ^{99m}Tc-Tx was primarily in the lung was demonstrated on the SPECT-CT overlays. As shown in FIG. 6, there was uniform distribution of ^{99m}Tc-Tx in lung when the radioactivity was viewed with low intensity scale settings. A speckled pattern of higher binding of ^{99m}Tc-Tx was also observed. This component is represented separately in an identical field in FIG. 6 with higher intensity scaling. Of interest, the speckled areas were associated with the vasculature, and appeared to be at the level of the 5th or 6th branch of the pulmonary artery. A magnified overlay and shows the speckled regions of ^{99m}Tc-Tx uptake were associated with the vasculature.

(2) Biodistribution

Lung showed the highest accumulation of ^{99m}Tc-Tx, averaging 49±4% ID/g (6.7 μg dose). Liver was second at 3.3±0.3% D/g, while all other tissues were less than 0.7% ID/g. Blood was only 0.4±0.1% ID/g for this dose; total blood activity accounted for only 4.7±1.4% ID. By comparison, the lung accumulation was reduced to 22.5% ID/g in the rat injected with the same dose of ^{99m}Tc-Tx diluted with unlabeled Tx3.833. For this animal, the blood levels were increased to 4.1% ID/g, with total blood activity accounting for 48% ID. The lung accumulation for the lowest dose of ^{99m}Tc-Tx (1.3 μg) was 49±8% ID/g, with liver levels of 2.0±0.6% ID/g. Results from Exp. 2 at 23 hr showed a high level of lung retention for the higher dose of ^{99m}Tc-Tx (28 μg), averaging 30±8% ID/g.

### 15. Example 15

### Complement Facilitates Infection of the Liver

Two groups of mice both received Ad5FF/6His. The normal fiber structure was replaced by fibritin in the vector. The first group received a dose of 4E10 v.p, and the second group received a dose of 4E9 v.p. The first group has two sets of mice, one set are C3 knockouts, the other set are wild type (FIG. 23). At the dosage level of 4E10, both sets are equal during the first ten days of dosing. However, wild type mice eliminate the liver infected cells due to the immune response. This does not happen with the C3 knockout mice.

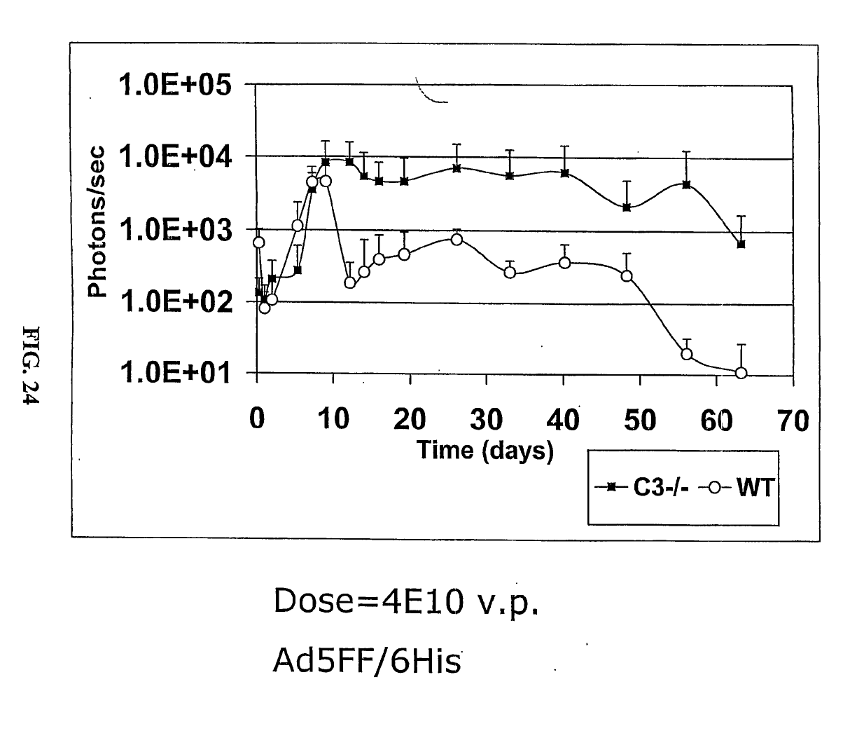

The second group of mice that receive a dose of 4E9 v.p. of Ad5FF/6His (FIG. 24). Again, there are two sets of mice, one set are C3 knockouts and the other are wild type mice. The wild type mice initially display higher levels of infection which tapers off, while the C3 knockout mice show steady levels of infection with no marked decrease. This shows that complement facilitates infection of the liver. The significance of the Ad5FF/6His is that it shows that complement is important even in the absence of normal infection mechanisms via CAR (coxsackie adenoviral receptor) (Zinn et al., Gene Therapy 11:1482-86, 2004, herein incorporated by reference in its entirety for its teaching regarding complement). However, with regular infection via CAR, the complement is also very important as discussed under Example 1.

### 16. Example 16

### Genetic Strategy to Decrease Complement Activation and Thereby Reduce Toxicity and Immune Response to Gene Therapy Vectors

a) Insertion into Hexon HVR2 and HVR5.

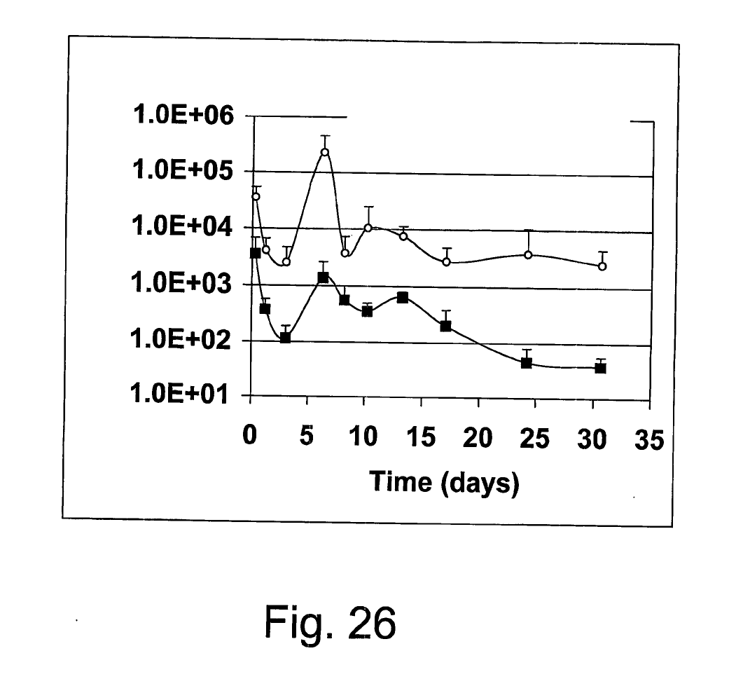

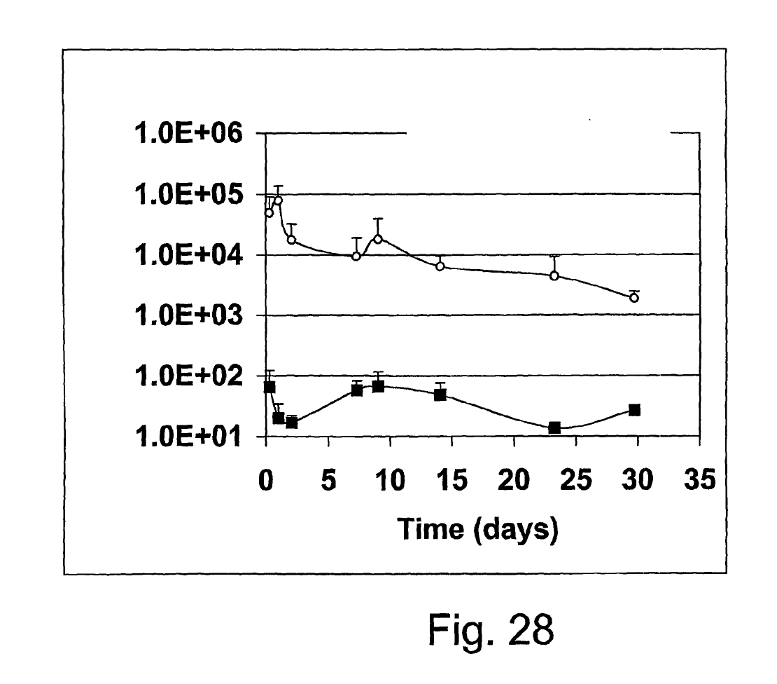

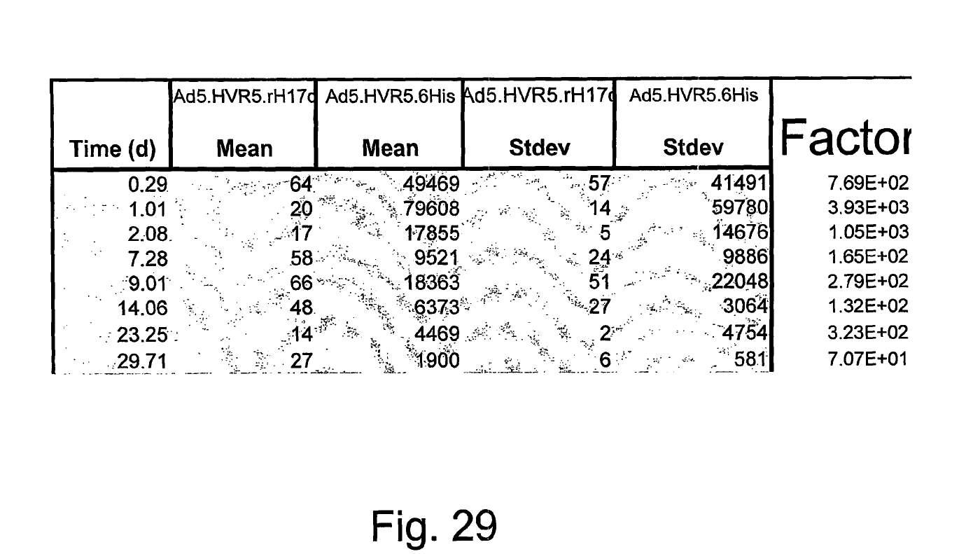

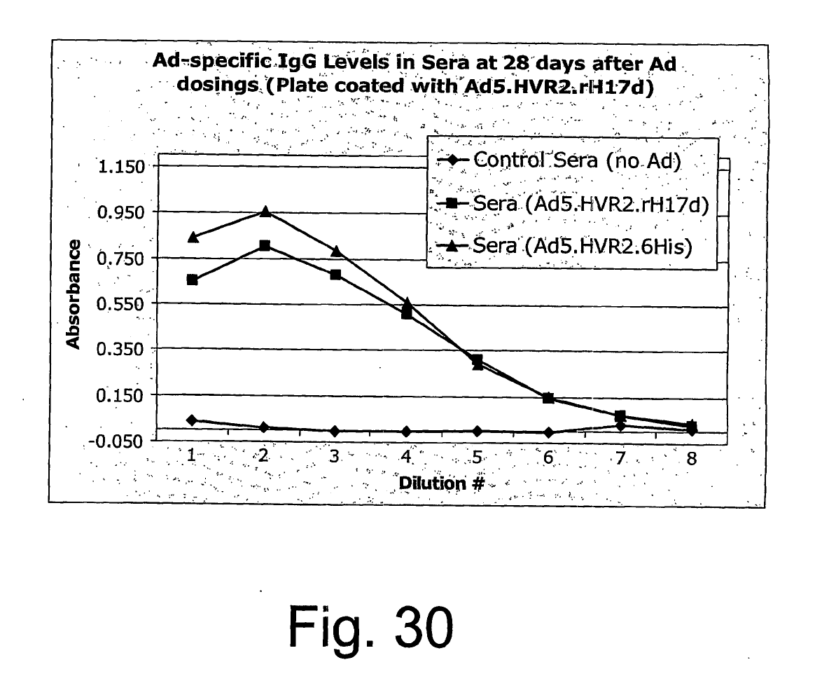

The complement pathway plays an important role in liver transfection by Adenovirus. As the process involves coating of the viral particles by activated complement factors, and thereby undesired consequences, including liver transfection and inflammation, it was desirable to genetically engineer a virus that displayed numerous copies of a protein that would down-modulate the complement pathway. Rux et al taught that various serotypes of adenoviruses could be grouped according to sequences variations in what he referred to as the hypervariable regions (HVR) of the hexon structural protein, naming them from HVR1 to HVR7. Therefore, it was reasoned that a peptide insert to down-modulating complement would be tolerated in these regions, and because of the large number of hexon making up the Ad vector (240 trimeric hexons), the total number of inserts to be displayed would be advantageous, that is 240×3=720 copies. The peptide insert was produced by genetic modification of the hexon DNA sequence, with the new protein insert-hexon chimera produced during replication and packaging of the Ad vector. The protein that was selected as the insert was a modified version of rH17d, itself a modification of Sh-TOR-ed1, a sequence with similarity to the beta-chain of human and mouse C4b. This sequence was known to down-modulate complement. The 36 amino acid protein sequence (reference herein as rH17d′) “LGS-HEVKIKHFSPY-HEVKIKHFSPY-GS-HHHHHH-LGS”(SEQ ID NO: 9) was inserted separately, in the HVR2 and HVR5 regions of the Ad5 hexon, using established cloning procedures (Example 17). The starting genetic code for these protein inserts was identical to the genetic code of the inserts in the new Ad5 vectors, as presented in FIGS. 26-29. Two additional control Ad5 vectors were prepared, with inserts encoding the 12 amino acid sequence LGS-HHHHHH-LGS (SEQ ID NO: 12), and referenced as “6His”. The four new Ad5 vectors that were prepared were subjected to further evaluations, as described herein.

b) SDS for New Ad Vectors

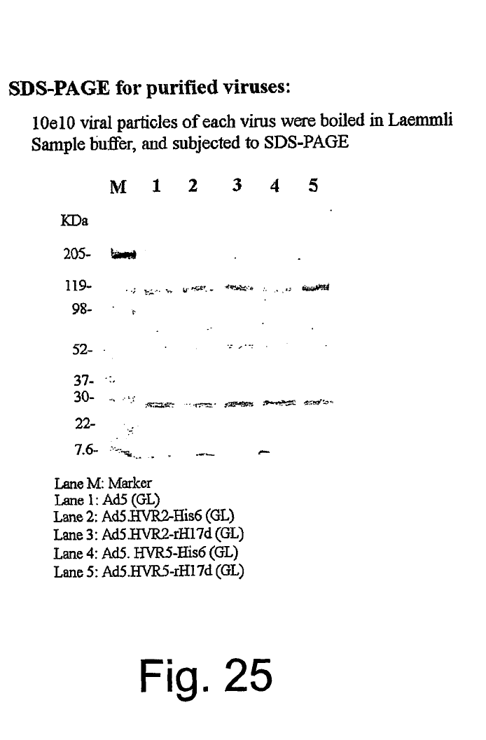

As shown in FIG. 25, the new Ad vectors encoded proteins that were of the correct size as determined by SDS polyacrylamide gel electrophoresis (PAGE).

As shown in FIGS. 26-29, the Ad5 vectors with rH17d′ inserts (in either HVR2 or HVR5) showed significantly less liver luciferase expression for the same 4×10⁹ dose of viral particles, as compared with Ad5 vectors with the 6His inserts. These data indicate that the rH17d′ insert was inhibiting liver transfection that would otherwise be found in these animals with active complment pathway, and as shown for the Ad5 vectors with 6His inserts in the same HVR regions.

c) Antibody Tests

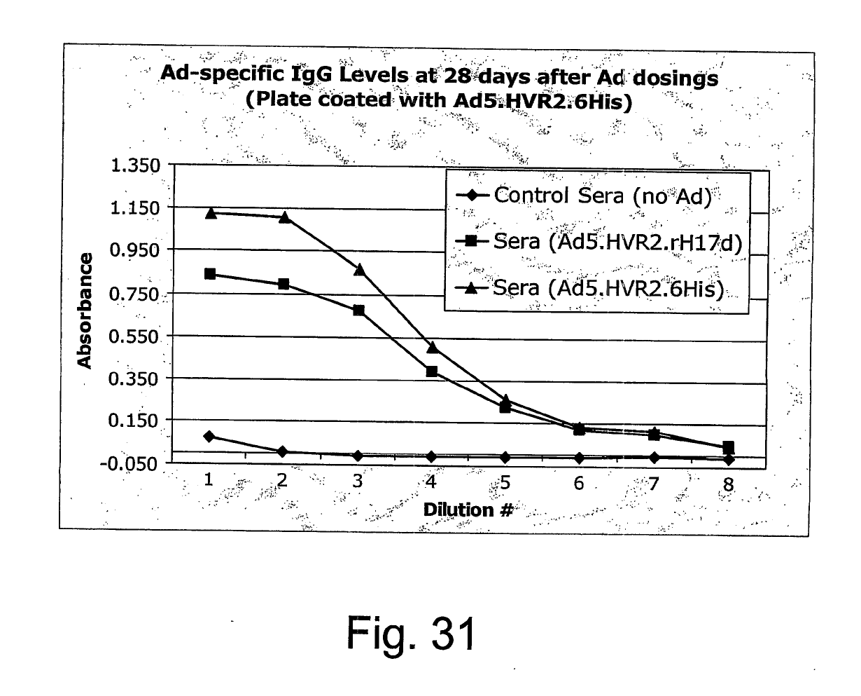

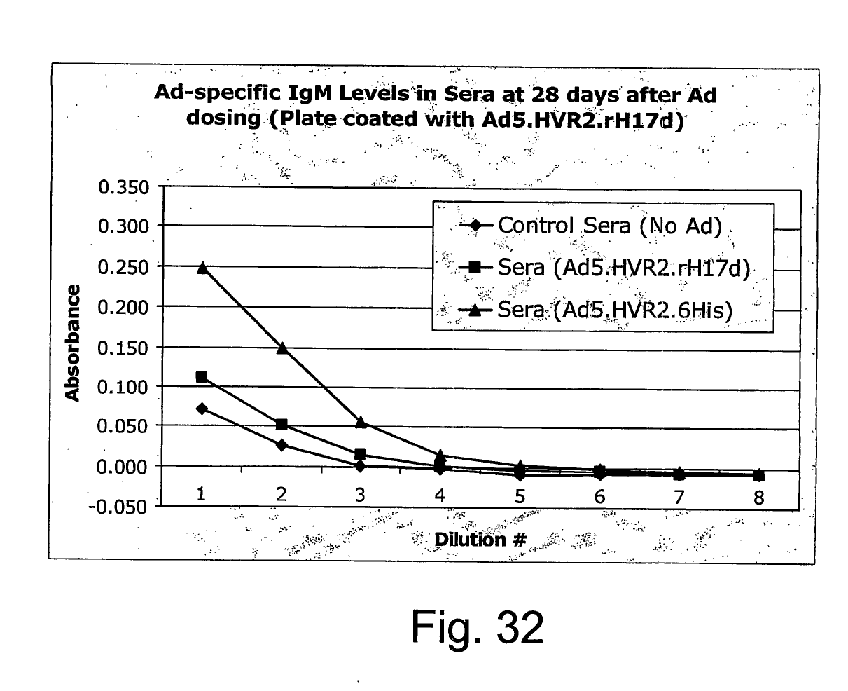

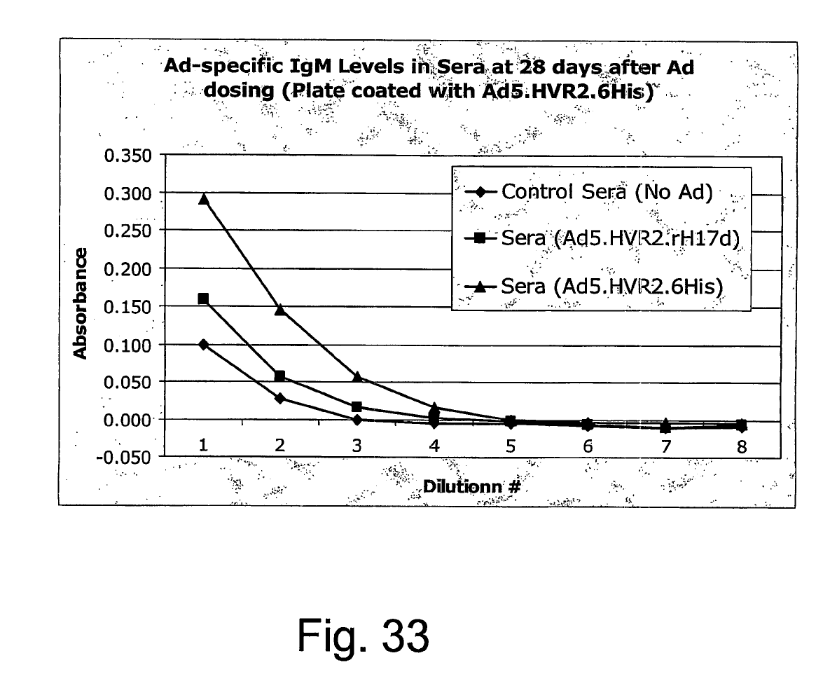

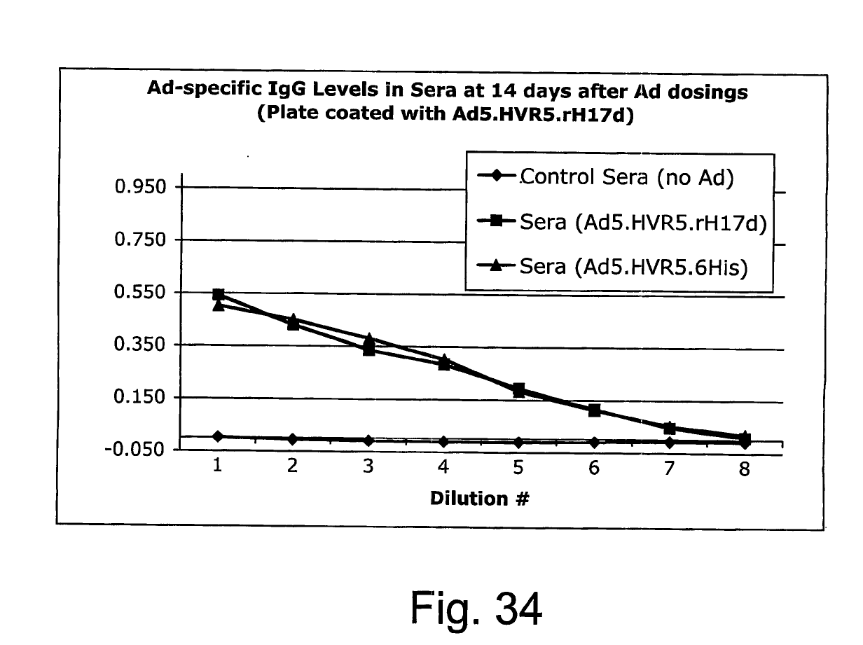

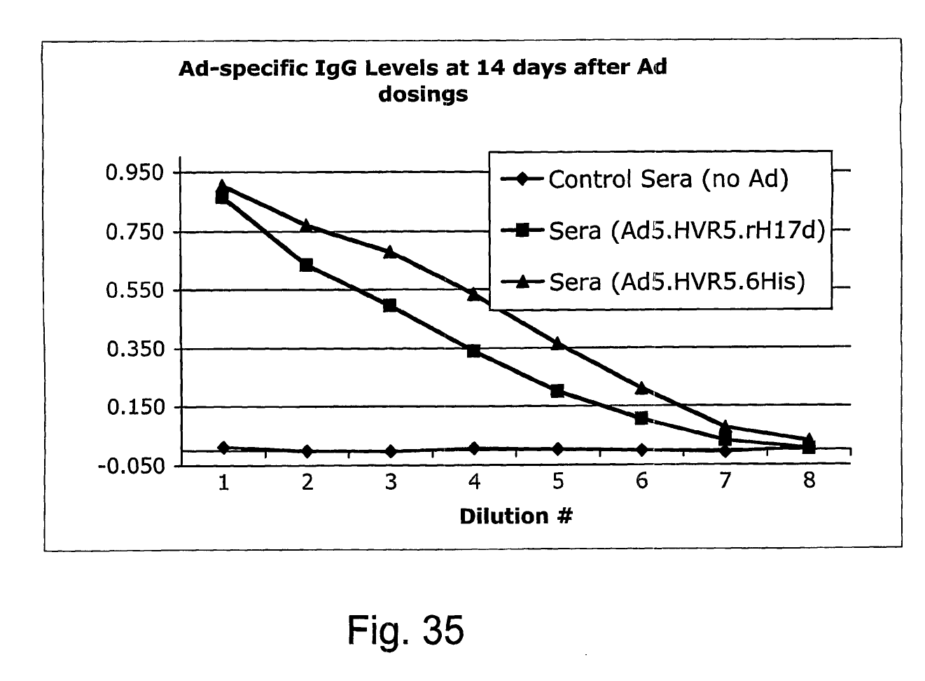

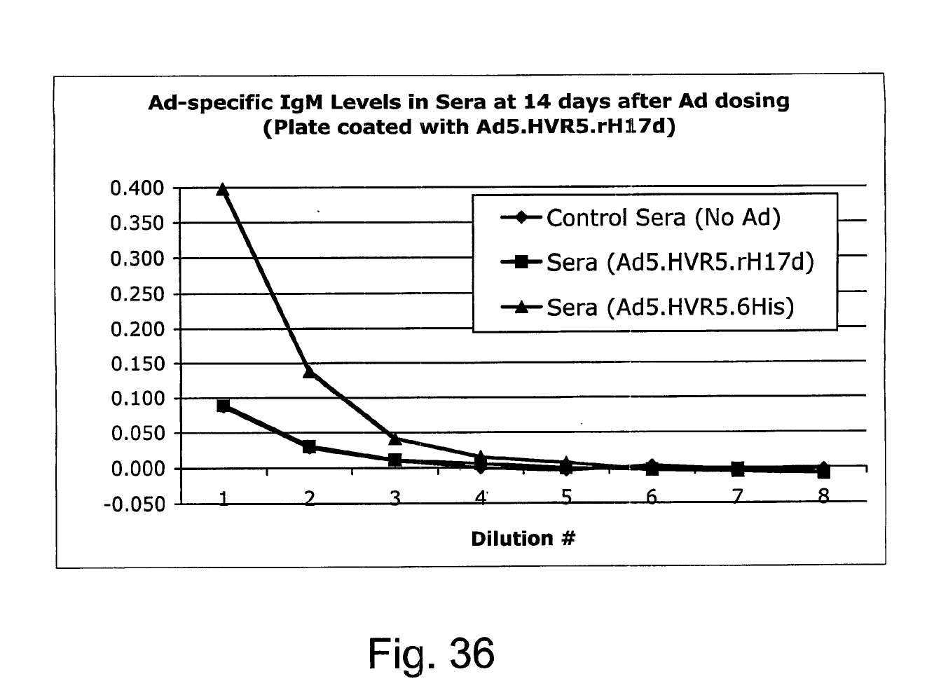

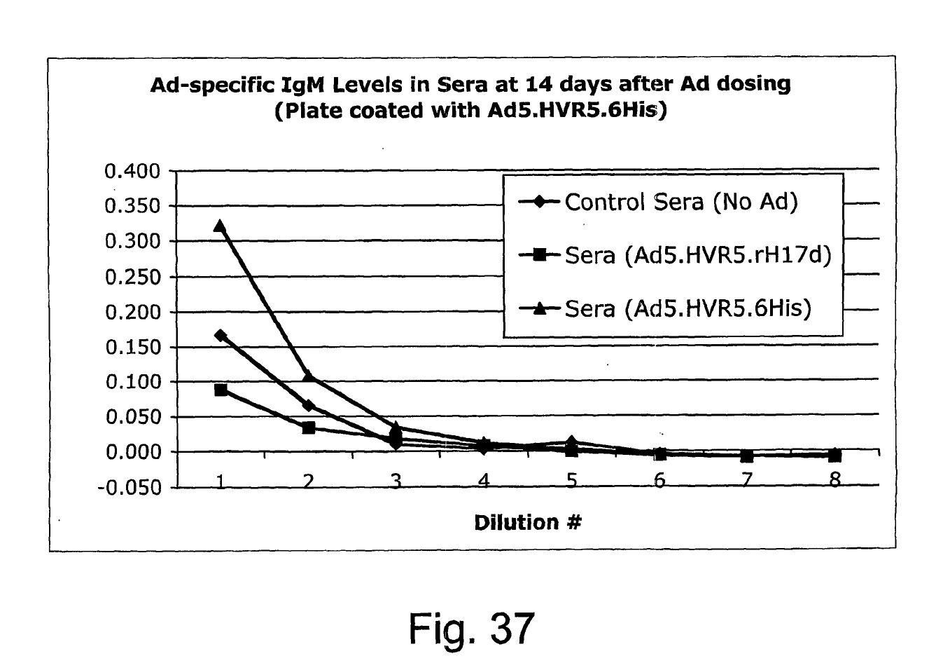

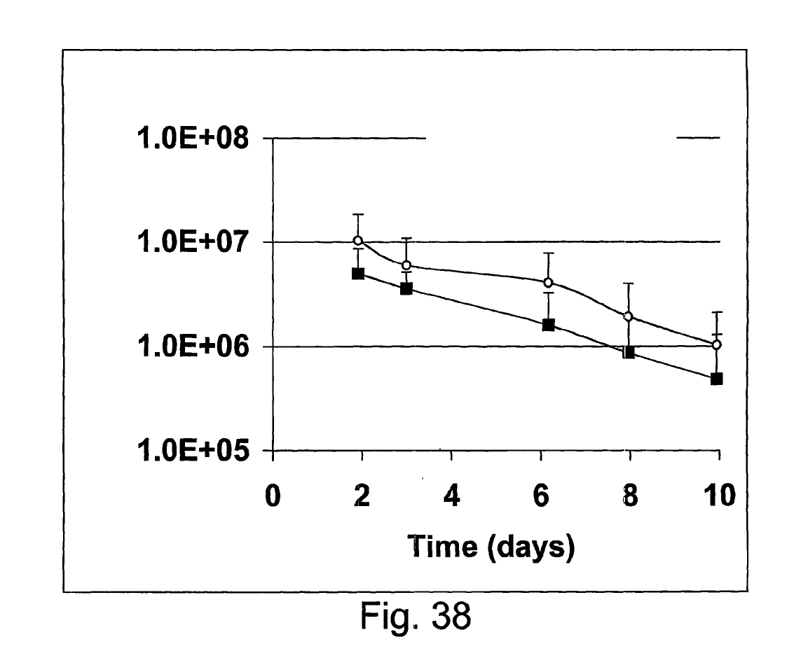

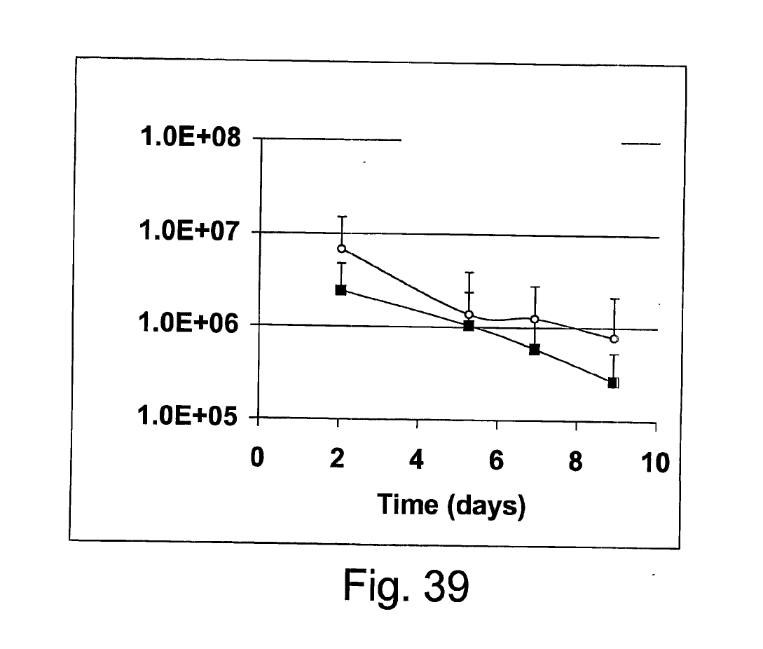

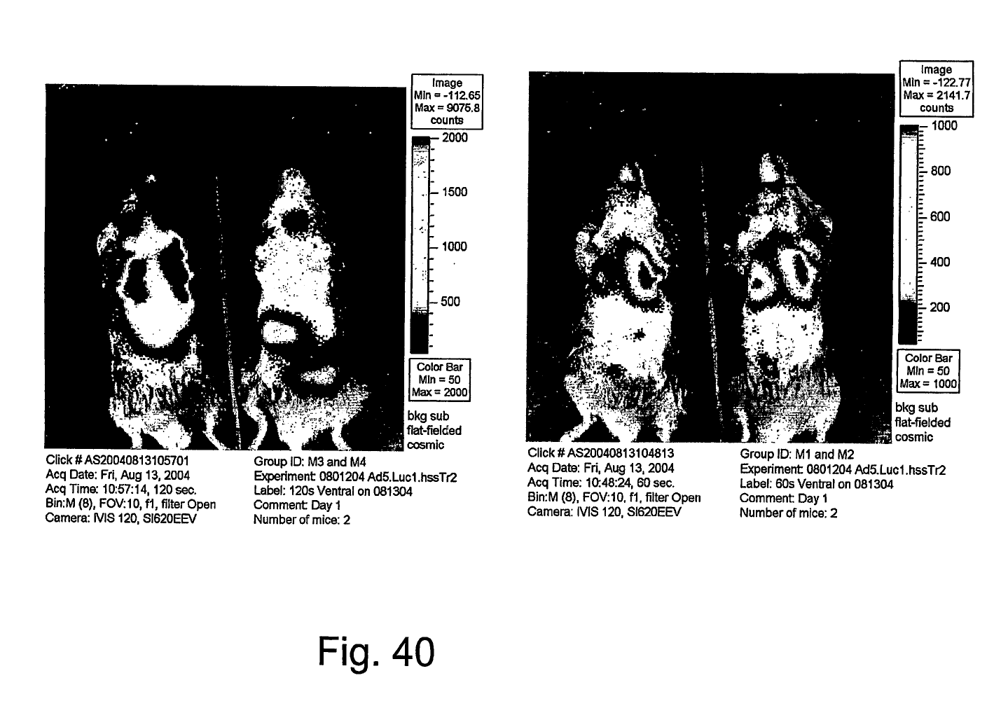

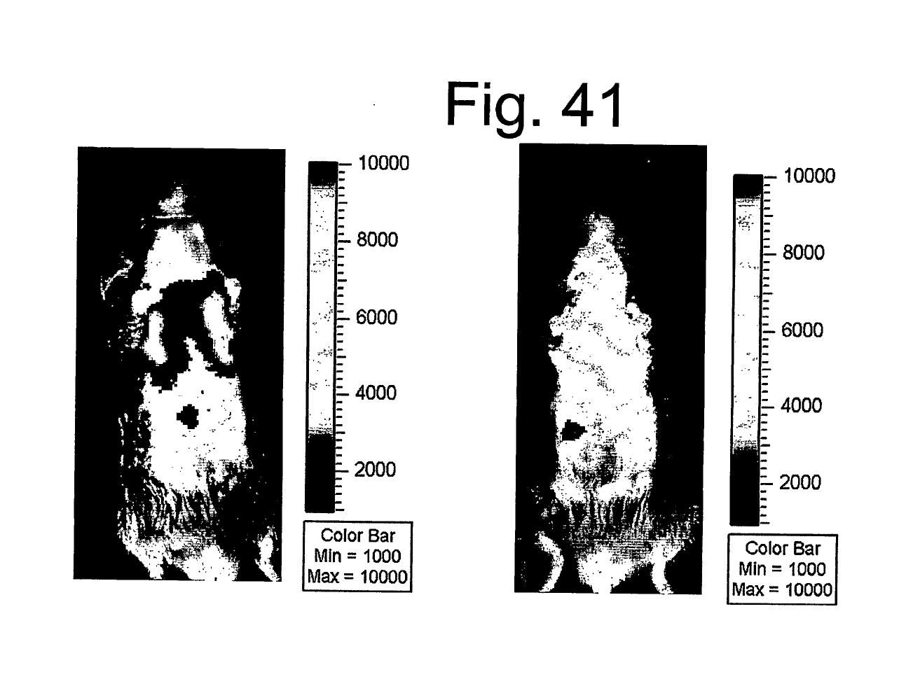

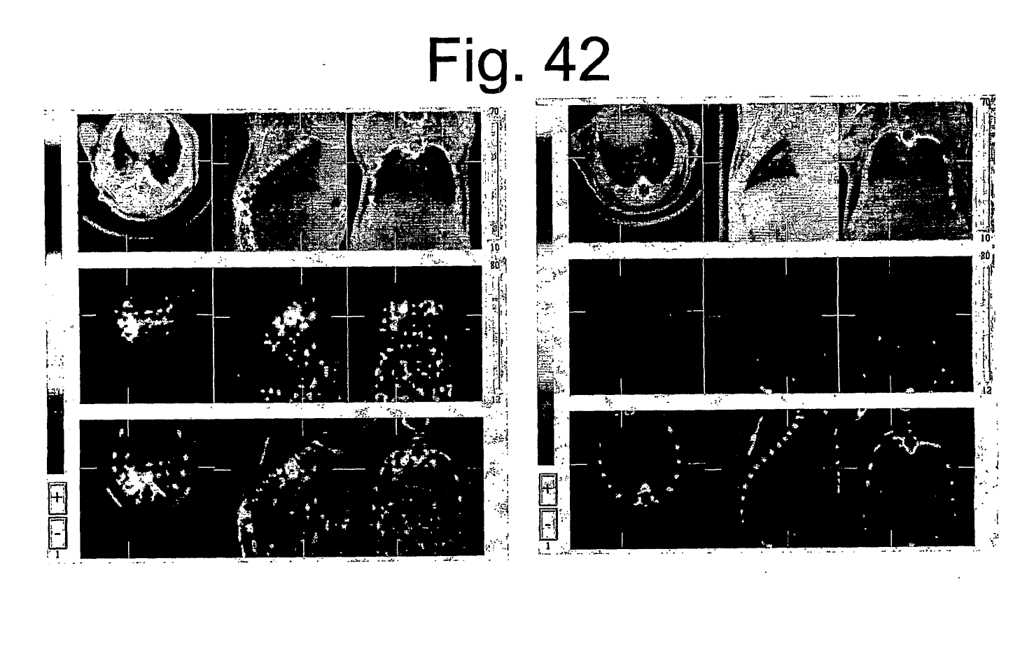

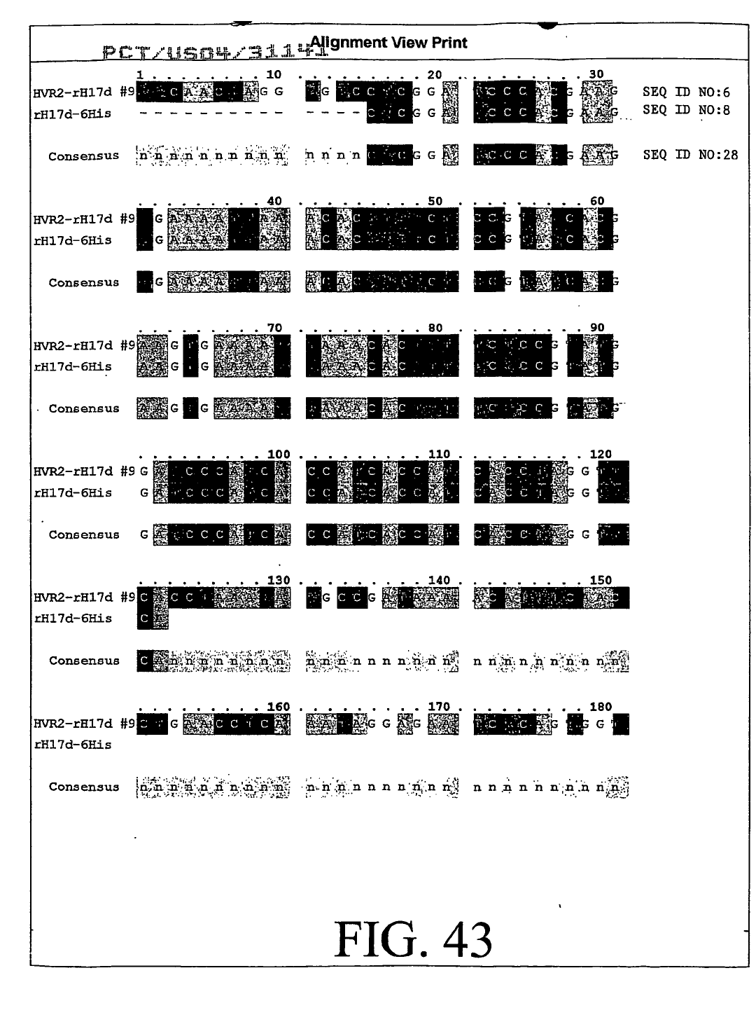

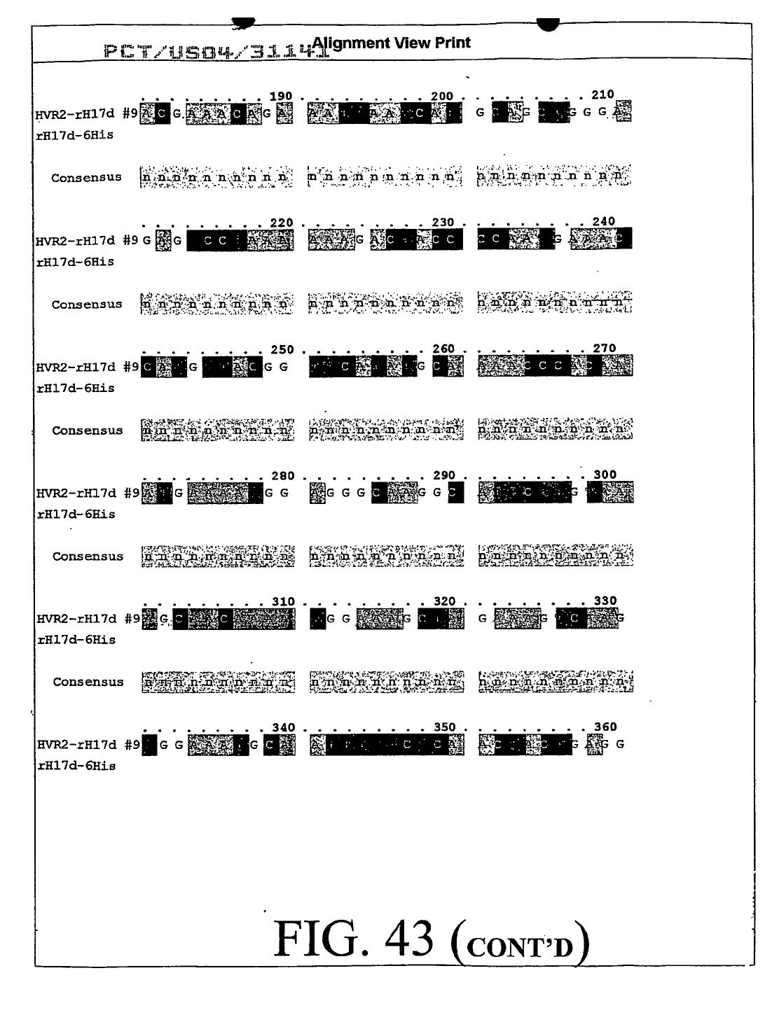

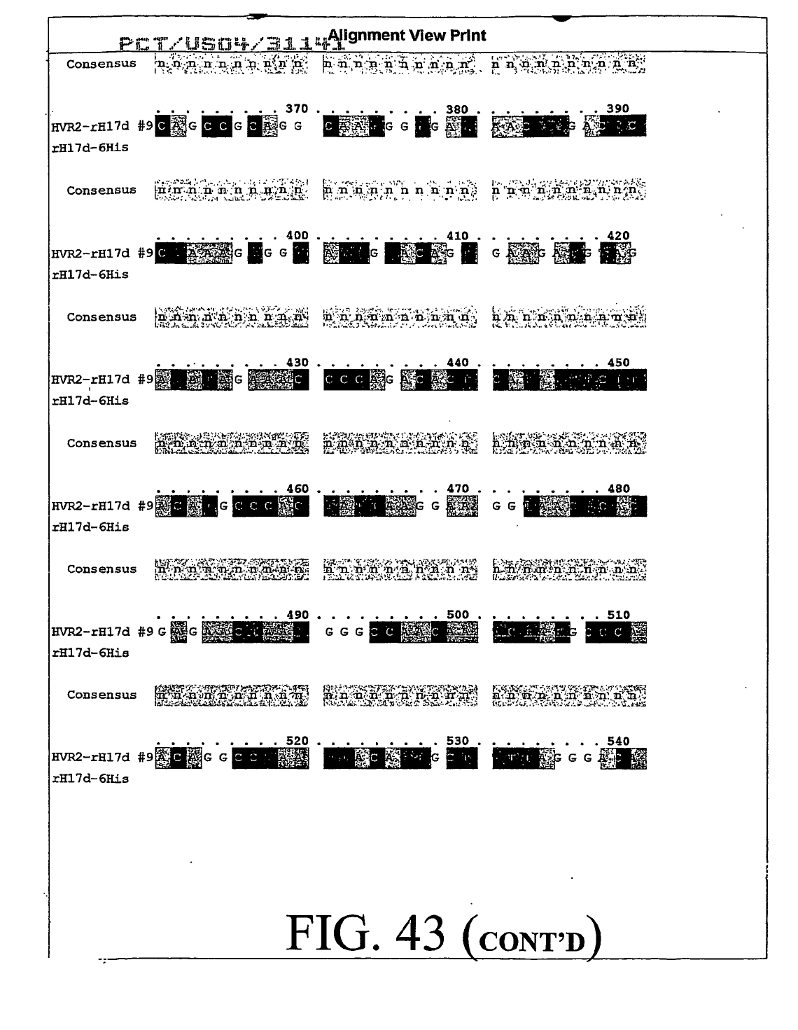

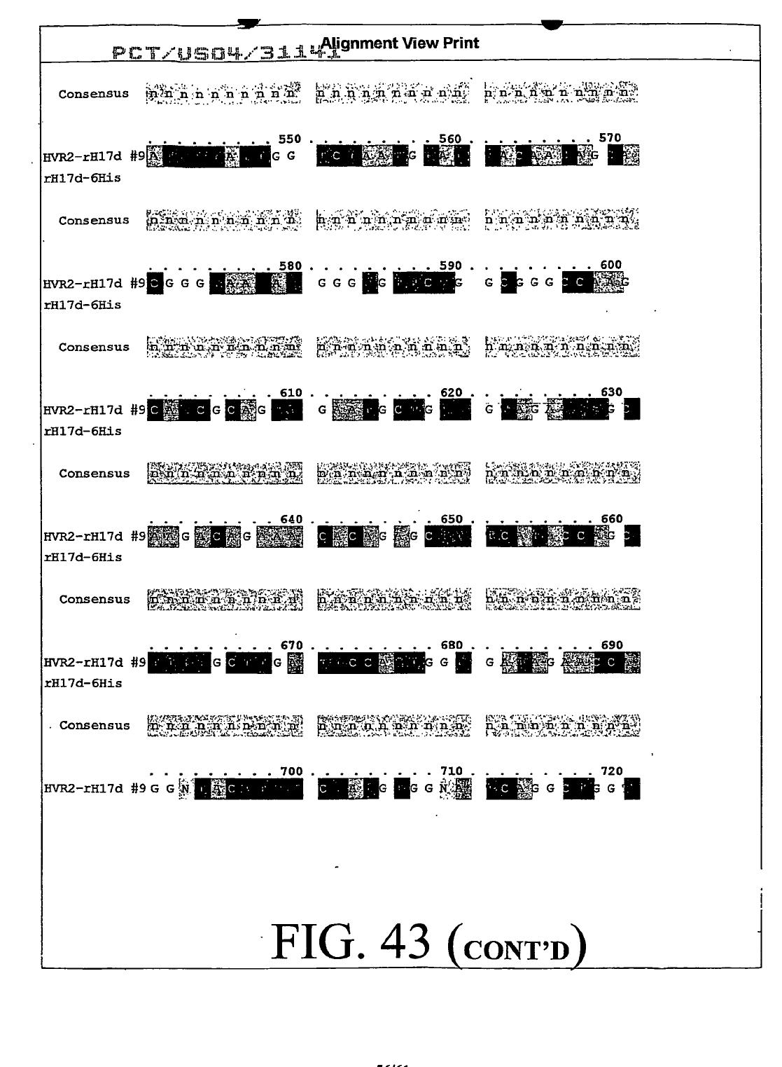

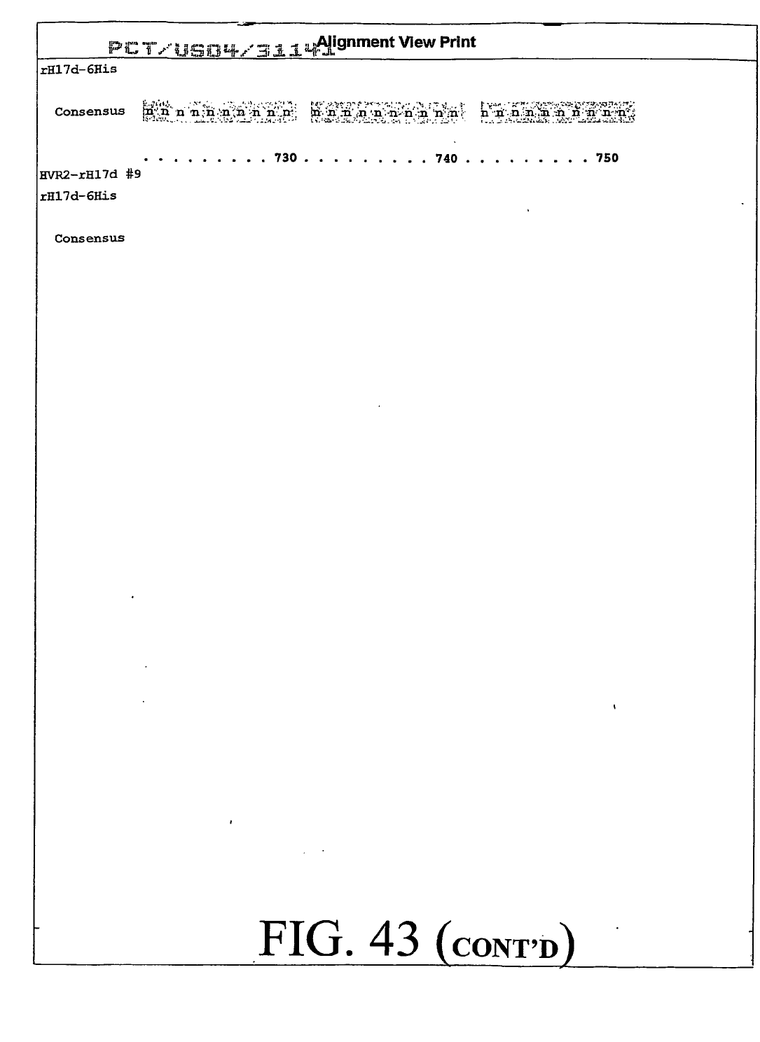

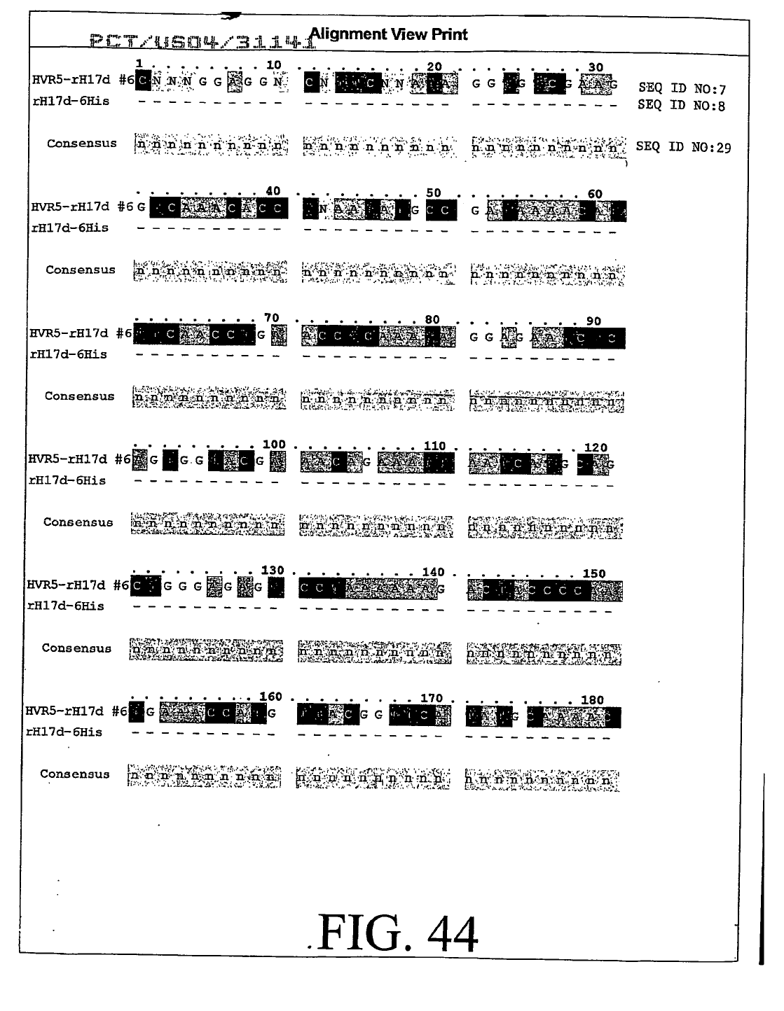

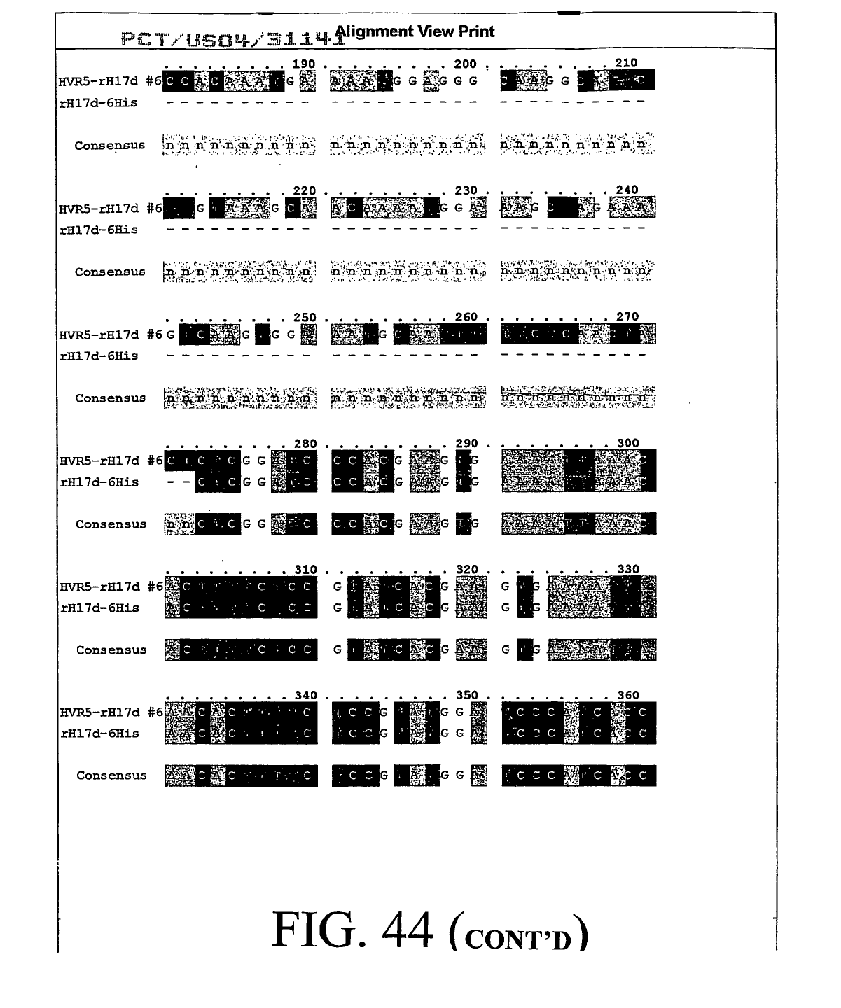

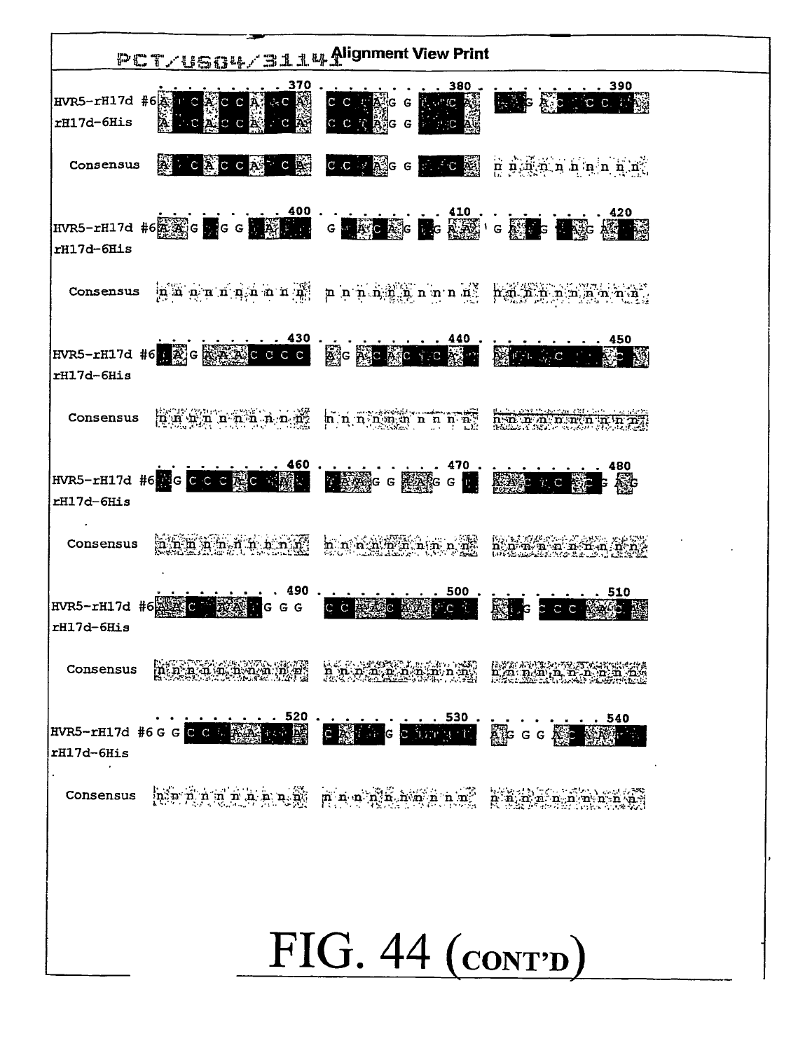

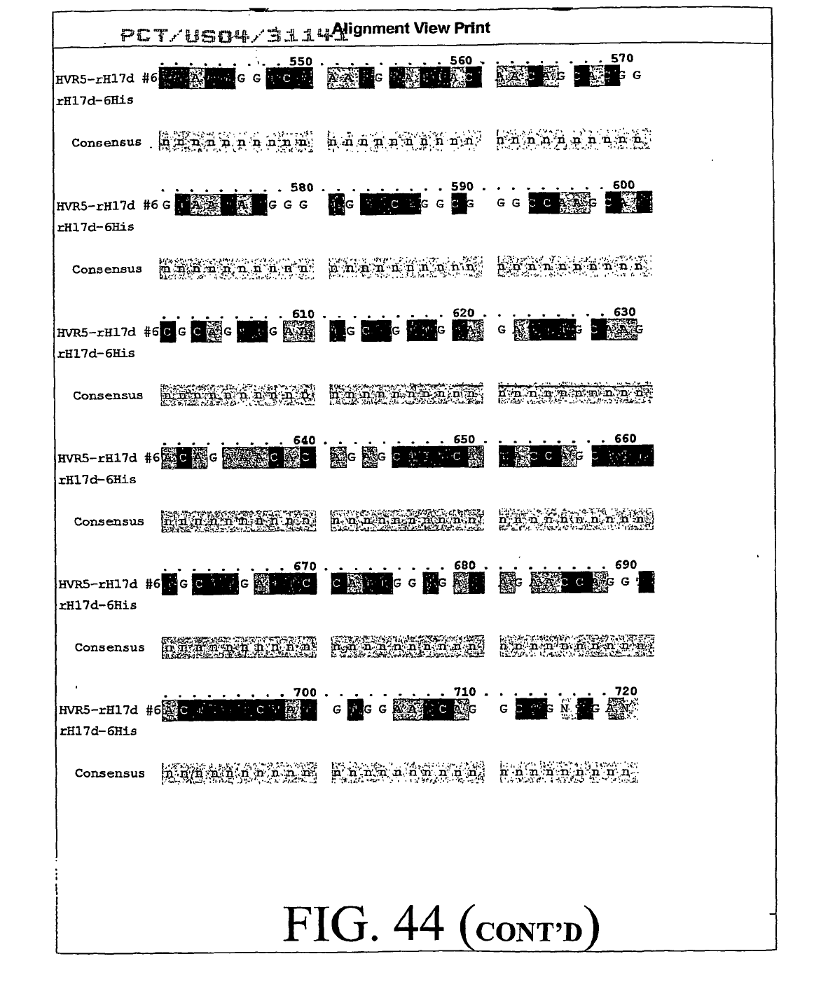

Similarly, as shown in FIGS. 30-37, the levels of IgG and IgM antibodies in sera, as measured under various conditions and time points, was less in animals injected with the Ad5 containing the rH17d′ inserts, as compared with animals injected with the Ad5 with the 6His inserts.

(1) Methods

Anti-IgG and anti-IgM antibody levels in sera from mice injected with Ad5.HVR2-rH17d′, Ad5.HVR2-6His, or no injection controls were subjected to an antibody titer test. Sera samples from the mice were pooled (n=6/group). Therefore, 3 pooled samples were evaluated. Preparation of Ad5.HVR2-rH17d′ and Ad5.HVR2-6His are in Table 1:

TABLE 1
Preparation of Ad vectors for plate assay

| | | | | | Diluted | | | | | |
|---|---|---|---|---|---|---|---|---|---|---|
| | Stock | Stock | Stock | Total | Needed | Total | PBS | Diluted | Per | Total |
| Ad Vector | v.p./ml | volume-ml | Total v.p. | Wells | Volume | Volume | Needed | Total v.p./ml | Well | v.p./ml |
| Ad5.HVR2-rH17d | 1.96E+12 | 0.055 | 1.08E+11 | 96 | 9.600 | 10.000 | 9.945 | 1.08E+10 | 0.100 | 1.08E+09 |
| Ad5.HVR2-6His | 5.44E+12 | 0.020 | 1.09E+11 | 96 | 9.600 | 10.000 | 9.980 | 1.09E+10 | 0.100 | 1.09E+09 |

Dilutions were first done in a 1:50 ratio, using BS-BSA for dilutions. The diluted Ad vectors were then added to plates (Costar 3590) as indicated in 0.100 ml and incubated 5 hours at room temperature. Ad vectors were removed and washed with PBS/0.05% Tween (Core Facility), and the plate wells were blocked with 0.15 ml Borate saline (pH 8.4 with 1% BSA), for 1-1.5 hours, and then washed with PBS/0.05% Tween. The diluted pooled sera was added, and allowed to incubate overnight at 4 C. The pooled sera was then removed, washed with PBS/0.05% Tween, and 0.1 mL goat anti-IgG or goat anti-IgM (Southern Biotech) that was conjugated with alkaline phosphatase was added, and incubated for 4 hours at room temperature. It was washed again with PBS/0.05% Tween. 0.1 mL of the substrate for alkaline phosphatase was added (p-nitrophenylphosphate (Sigma), 1 mg/ml, dissolved 4 tables in 20 ml PBS/0.05% Tween), incubated for 20 minutes, and read on a plate reader at 405 nm.

d) Luc Expression in A427 Tumors.

For direct injections of human non small cell lung (A427) tumors that were growing s.c. in nude mice, similar levels of luciferase expression were measured over time in the tumors (overall not statistically different between groups), indicating that tumor infection was similar for the rH17d′ and 6his inserts. Therefore, these data support the concept that the complement system was not important for the tumor transfection, as one determined for liver transfection.

### 17. Example 17

### Identification of Sites in Adenovirus Hexon for Foreign Peptide Incorporation

Construction of adenoviral vectors. To incorporate 6-His epitope into the HVRs of hexon, hexon fragments were obtained containing sequences that encode 6-His and the spacers (Lys-Gly-Ser, SEQ ID NO: 13) in different HVRs via three-step polymerase chain reaction (PCR). For example, to obtain 6-His insertion in HVR2 (HVR2-6-His), using Ad5 hexon as template, fragment 2L was first amplified (left to HVR2 insertion) with primers CCT ACG CAC GAC GTG ACC ACA G (primer L, Dra III, SEQ ID NO: 14) and TGA ACC TAG GTG ATG GTG ATG GTG ATG GGA TCC GAG GAC ACC TAT TTG AAT ACC CTC CTT TG (primer HVR2-6-His, SEQ ID NO: 15), and fragment 2R (right to HVR2 insertion) with primers CTC GGA TCC CAT CAC CAT CAC CAT CAC CTA GGT TCA CCT AAA TAT GCC GAT AAA ACA TTT C (primer HVR2-6-His, SEQ ID NO: 16) and CTA GGG AGC TCT GCA GAA CCA TG (primer R, Sac I, SEQ ID NO: 17). After purification of fragments 2L and 2R, 25-50 ng of each fragment (equal molar ratio) was mixed and used as template and primers for second step of PCR, resulting insertion of sequences encoding 6-His and the spacers into HVR2. Next, primer L and primer R were added into the tubes, and a third step of PCR was used to amplify the HVR2-6-His fragment. Other insertions were obtained in the same way with corresponding HVR-6-His primers. These primers are: primer HVR3-6-His (SEQ ID NO: 18), as TGA ACC TAG GTG ATG GTG ATG GTG ATG GGA TCC GAG TTC GTA CCA CTG AGA TTC TCC TAT, primer HVR3-6-His, (SEQ ID NO: 19) CTC GGA TCC CAT CAC CAT CAC CAT CAC CTA GGT TCA ACT GAA ATT AAT CAT GCA GCT GGG, primer HVR5-6-His, (SEQ ID NO: 20) TGA ACC TAG GTG ATG GTG ATG GTG ATG GGA TCC GAG AGT AGT TGA GAA AAA TTG CAT TTC C, primer HVR5-6-His, (SEQ ID NO: 21) CTC GGA TCC CAT CAC CAT CAC CAT CAC CTA GGT TCA TTG ACT CCT AAA GTG GTA TTG TAC, primer HVR6-6-His, (SEQ ID NO: 22) TGA ACC TAG GTG ATG GTG ATG GTG ATG GGA TCC GAG AGT GGG CAT GTA AGA AAT ATG AGT G, primer HVR6-6-His, (SEQ ID NO: 23) CTC GGA TCC CAT CAC CAT CAC CAT CAC CTA GGT TCA AAC TCA CGA GAA CTA ATG GGC C, primer HVR7a-6-His (SEQ ID NO: 24) TGA ACC TAG GTG ATG GTG ATG GTG ATG GGA TCC GAG AGG TTT TAC CTT GGT AAG AGT CTC, primer HVR7a-6-His (SEQ ID NO: 25) CTC GGA TCC CAT CAC CAT CAC CAT CAC CTA GGT TCA TGG GAA AAA GAT GCT ACA GAA TTT TC, primer HVR7b-6-His, (SEQ ID NO: 26) TGA ACC TAG GTG ATG GTG ATG GTG ATG GGA TCC GAG TGG AAA GCA GTA ATT TGG AAG TTC, primer HVR7b-6-His (SEQ ID NO: 27) CTC GGA TCC CAT CAC CAT CAC CAT CAC CTA GGT TCA AAT AAT TTT GCC ATG GAA ATC AAT CTA.

The hexon fragments containing 6-His epitope obtained above were purified and subcloned into Ad5 hexon shuttle vector H5/pH5S with Dra III and Sac I. The resultant shuttle plasmids were named as HVR2-6HIS/pH5S, HVR3-His6/pH5S, HVR5-His₆/pH5S, HVR6-His₆/pH5S, HVR7a-His₆/pH5S, and HVR7b-His₆/pH5S, respectively. To create Ad5 vector containing His₆ epitopes in the HVRs of hexon, these plasmids were digested with EcoR I and Pme I, and the fragments containing the homologous recombination regions and the hexon genes were purified, then recombined with Swa I-digested backbone Ad5 vector that lacks the hexon gene pAd5/ΔH5 in E. Coli BJ5183. The resulted clones were designated as pAd5/HVR2-6-His, pAd5/HVR3-6-His, pAd5/HVR5-6-His, pAd5/HVR6-6-His, pAd5/HVR7a-6-His, and pAd5/HVR7b-6-His, all of which contain green fluorescence protein (GFP) gene and firefly luciferase (Luc) gene in E1 region. The constructs were confirmed by restriction digestions and sequencing.

To rescue viruses, these modified plasmids were digested with Pac I, and 2 μg of each purified DNA were transfected into the Ad-E1 expressing 293 cells grown in 60-mm dishes using Superfect (Qiagen). After plaques were formed, they were processed for large-scale proliferation in 293 cells, followed by purification with CsCl gradient centrifugation.

SDS-PAGE and western blotting. 10¹⁰VPs of each CsCl-purified virus were dissolved in Laemmli sample buffer without boiling, and separated on 4-15% gradient SDS polyacrylamide gels (SDS-PAGE). The gels were either stained with Gelcode® Blue Stain Reagent (Pierce) according to the protocol from the manufacturer, or transferred to nitrocellulose membrane (Bio-Rad). The membrane was processed to western blotting with either anti-His-Tag monoclonal antibody or anti-hexon polyclonal antibody.

ELISA. ELISA binding assay was performed essentially as described. In brief, different amount of viruses ranging from 4×10⁶ to 9×10⁹ VPs were immobilized on wells of a 96-well plate (Nunc Maxisorp) by overnight incubation in 100 ul/well of 100 mM Carbonate buffer (pH 9.5) at 4° C. After extensive washes with 0.05% Tween-20 in Tris-buffered saline (TBS) and blocking with blocking solution (2% bovine serum albumin (BSA) and 0.05% Tween-20 in TBS), the immobilized viruses were incubated with anti-His-Tag monoclonal antibody (Chemicon) for 2 hours at room temperature, followed by Alkaline Phosphatase (AP)-conjugated goat anti-mouse antibody incubation. Color reaction was performed with p-nitrophenyl phosphate (Sigma) as recommended by the manufacturer, and absorbance at 405 nm (OD405) was obtained with a microplate reader (Molecular Devices).

Gene transfer assay. 1) Gene transfer assay in Hela cells, U118MG cells, andU118MG.HissFv.Rec cells. Gene transfer efficacy of the viruses was evaluated by lucifease activity essentially as described herein. In brief, Hela cells, U118MG cells, and U118MG.HissFv.Rec cells were plated in 24-well plates with a density of 10⁵ cells per well the day before infection. Cells were infected at Multiplicity Of Infections (MOIs) of 1, 10, and 100 VPs/cell in triplicates. 24 hours later, the cells were lysed in 250 μl per well of reporter lysis buffer (RLB) (Promega), followed by one freeze/thaw cycle. Five μl of each sample was used to measure the luciferase activity with a luciferase assay kit (Promega) and a luminometer (Berthold, Gaithersburg, Md.).

2) Gene transfer assay in transient artificial system. To establish a transient system expressing artificial receptor for His-Tag, U118MG cells were infected with Ad5.MK.AR that encodes anti-His-Tag single chain antibody, the artificial receptor for His tag (AR), in the E1 region under the control of CMV promoter (Param's paper) with MOI=300, and cultured for 3 days to allow the AR to express. An E1 deleted vector, Ad5.E1dd, was used as control. These cells were then infected with the 6-His-containing viruses at MOI=100, and their gene transfer efficacy was measured 24 hours later as described above.

Thermostability assay. To test thermostability of these viruses, viruses equivalent to MOI 100 were incubated at 45° C. for different time intervals before infecting Hela cells. Luciferase activity in infected cells was analyzed 24 hours post-infection as described above.

Throughout this application, various publications are referenced. The disclosures of these publications in their entireties are hereby incorporated by reference into this application in order to more fully describe the state of the art to which this invention pertains.

### REFERENCES

Abrams M J, Juweid M, tenKate C L Schwartz D A, Hauser M M, Gaul F E, Fuccello A J, Rubin R H, Strauss H W, and Fischman A J, Technetium-99m-human polyclonal IgG radiolabeled via the hydrazine nicotinamide derivative for imaging focal sites of infection in rats. J Nucl Med 31:2022-8, 1990.
Anderson C J, Dehdashti F, Cutler P D, Schwarz S W, Laforest R, Bass L A, Lewis J S, and McCarthy D W, 64Cu-TETA-octreotide as a PET imaging agent for patients with neuroendocrine tumors. J Nucl Med 42:213-21, 2001.
Aoki M, Kanamori M, Yudoh K, Ohmori K, Yasuda T, and Kimura T, Effects of vascular endothelial growth factor and E-selectin on angiogenesis in the murine metastatic RCT sarcoma. Tumour Biol 22:239-46, 2001.
Auricchio A, Zhou R, Wilson J M, and Glickson J D, In vivo detection of gene expression in liver by 31P nuclear magnetic resonance spectroscopy employing creatine kinase as a marker gene. Proc Natl Acad Sci U S A 98:5205-10, 2001.
Becker A, Hessenius C, Bhargava S, Grotzinger C, Licha K, Schneider-Mergener J, Wiedenmann B, and Semmler W, Cyanine dye labeled vasoactive intestinal peptide and somatostatin analog for optical detection of gastroenteropancreatic tumors. Ann N Y Acad Sci 921:275-8, 2000.
Becker A, Hessenius C, Licha K, Ebert B, Sukowski U, Semmler W, Wiedenmann B, and Grotzinger C, Receptor-targeted optical imaging of tumors with near-infrared fluorescent ligands. Nat Biotechnol 19:327-31, 2001.
Bennett J, Duan D, Engelhardt J F, and Maguire A M, Real-time, noninvasive in vivo assessment of adeno-associated virus-mediated retinal transduction. Invest Ophthalmol Vis Sci 38:2857-63, 1997.
Bergelson J M, Cunningham J A, Droguett G, Kurt-Jones E A, Krithivas A, Hong J S, Horwitz M S, Crowell R L, and Finberg R W, Isolation of a common receptor for Coxsackie B viruses and adenoviruses 2 and 5. Science 275:1320-3, 1997.
Biermann V, Volpers C, Hussmann S, Stock A, Kewes H, Schiedner G, Herrmann A, and Kochanek S, Targeting of high-capacity adenoviral vectors. Hum Gene Ther 12:1757-69, 2001.
Blasberg R G and Tjuvajev J G, Herpes simplex virus thymidine kinase as a marker/reporter gene for PET imaging of gene therapy. Q J Nucl Med 43:163-9, 1999.
Boland A, Ricard M, Opolon P, Bidart J M, Yeh P, Filetti S, Schlumberger M, and Perricaudet M, Adenovirus-mediated transfer of the thyroid sodium/iodide symporter gene into tumors for a targeted radiotherapy. Cancer Res 60:3484-92, 2000.
Breeman W A, de Jong M, Kwekkeboom D J, Valkema R, Bakker W H, Kooij P P, Visser T J, and Krenning E P, Somatostatin receptor-mediated imaging and therapy: basic science, current knowledge, limitations and future perspectives. Eur J Nucl Med 28:1421-9, 2001.
Bremer C and Weissleder R, In vivo imaging of gene expression. Acad Radiol 8:15-23, 2001.
Bugaj J E, Achilefu S, Dorshow R B, and Rajagopalan R, Novel fluorescent contrast agents for optical imaging of in vivo tumors based on a receptor-targeted dye-peptide conjugate platform. J Biomed Opt 6:122-33, 2001.
Bugaj J E, Erion J L, Johnson M A, Schmidt M A, and Srinivasan A, Radiotherapeutic efficacy of (153)Sm-CMDTPA-Tyr(3)-octreotate in tumor- bearing rats. Nucl Med Biol 28:327-34, 2001.
Caragine T A, Imai M, Frey A B, and Tomlinson S, Expression of rat complement control protein Crry on tumor cells inhibits rat natural killer cell-mediated cytotoxicity. Blood 100:3304-10, 2002.
Caragine T A, Okada N, Frey A B, and Tomlinson S, A tumor-expressed inhibitor of the early but not late complement lytic pathway enhances tumor growth in a rat model of human breast cancer. Cancer Res 62:1110-5, 2002.
Cascini G L, Cuccurullo V, Rambaldi P F, and Mansi L, [Immunological imaging using tagged octreotide]. Minerva Endocrinol 26:129-33, 2001.
Chatziioannou A, Kehagias D, Mourikis D, Antoniou A, Limouris G, Kaponis A, Kavatzas N, Tseleni S, and Vlachos L, Imaging and localization of pancreatic insulinomas. Clin Imaging 25:275-83, 2001.
Chaudhuri T R, Cao Z, LoBuglio A F, and Zinn K R, In vivo imaging of angiogenesis in breast cancer xenografts during therapy. Breast Cancer Res. Treat. 76:S 122, 2002.
Chaudhuri T R, Buchsbaum D, Mountz J, Rogers B E, Partridge E, and Zinn K R, Early detection of ovarian cancer by light-based imaging. Gynec Oncol 80:330, 2001.
Chaudhuri T R, Buchsbaum D, Mountz J, Rogers B E, Partridge E, and Zinn K R, Early detection of ovarian cancer by light-based imaging. Gynec Oncol 80:330, 2001.
Chaudhuri T R, Cao Z, Burford-Rodriquez C, Partridge E, and Zinn K R, A non-invasive approach for monitoring breast tumor cells during therapeutic intervention. Cancer Biother Radiopharm 17:205-212,2002.
Chaudhuri T R, Cao Z, Davis A J, Della Manna D, Rodriguez-Burford C, Robinson G D, Partridge E E, and Zinn K R, Treatment monitoring of microscopic ovarian cancer in live mice. Proc of ASCO 21a:887, 2002.
Chaudhuri T R, Cao Z, Della Manna D L, Rodriguez-Burford C, Partridge E E, and Zinn K R, A sensitive method for treatment monitoring of microscopic ovarian cancer in live mice. Mol Ther 5:S115, 2002.
Chaudhuri T R, Cao Z, Krasnykh V N, Stargel A V, Belousova N, Partridge E E, and Zinn K R, Blood-based Screening and Light Based Imaging for the Early Detection and Monitoring of Ovarian Cancer Xenografts. Technol Cancer Res Treat 2:171-80, 2003.
Chaudhuri T R, Cao Z, Ma Z, and Zinn K R, Optical imaging combined with blood-based screening by a new plasmid vector for the detection of ovarian cancer. Eur J Nucl Med Mol Imaging 29:S177, 2002.
Chaudhuri T R, Cao Z, Partridge E E, and Zinn K R, A comparative analyses of light-based and gamma-camera imaging to detect non-palpable ovarian cancer lesion in nude mice. Mol Ther 5:S270, 2002.
Chaudhuri T R, Cao Z, Partridge E E, and Zinn K R, A comparative analysis of optical and gamma camera imaging for non-invasive detection of ovarian cancer. Eur J Nucl Med Mol Imaging 29:S229,2002.
Chaudhuri T R, Cao Z, Partridge E E, and Zinn K R, Dual modality imaging for treatment monitoring of microscopic ovarian cancer in live mice. Eur J Nucl Med Mol Imaging 29:S178, 2002.
Chaudhuri T R, Cao Z, Rodriguez-Burford C, LoBuglio A F, and Zinn K R, In vivo optical imaging of angiogenesis in breast cancer xenografts during therapy. Mol Ther 5:S116, 2002.
Chaudhuri T R, Cao Z, Rogers B E, Partridge E E, and Zinn K R, Non-invasive light-based imaging of GFP-positive ovarian xenografts. Abstract Book p. 70, Annual Investigators' Workshop, 2001.
Chaudhuri T R, Krasnykh V, Belousova N, Zinn K R, Buchsbaum D, Mountz J, Curiel D, and Rogers B E, An ad-based strategy for imaging, radiotherapy, and enhanced tumor killing. Mol Ther 3:S64, 2001.
Chaudhuri T R, Krasnykh V N, Cao Z, and Zinn K R, Dualistic Genetic Reporter System for Early Diagnosis and Monitoring of Ovarian Cancer. Mol Ther 5:S207, 2002.
Chaudhuri T R, Mountz J, B E R, and Zinn K R, Imaging GFP-positive ovarian cancer cells in vitro during adriamycin treatment. Mol Ther 3:S217, 2001.
Chaudhuri T R, Mountz J, Rogers B E, Robinson G, Partridge E, and Zinn K R, Non-invasive light-based imaging of GFP-positive ovarian xenografts. Mol Ther 3:S404, 2001.
Chaudhuri T R, Mountz J M, Rogers B E, Partridge E E, and Zinn K R, Light-based imaging of green fluorescent protein-positive ovarian cancer xenografts during therapy. Gynecol Oncol 82:581-9, 2001.
Chaudhuri T R, Rogers B E, and Zinn K R, Non-invasive dual modality imaging of ovarian cancer in mice. Eur J Nucl Med 28:1179, 2001.
Chaudhuri T R, Rogers B E, Buchsbaum D J, Mountz J M, and Zinn K R, A noninvasive reporter system to image adenoviral-mediated gene transfer to ovarian cancer xenografts. Gynecol Oncol 83:432-8, 2001.
Chaudhuri T R, Rogers B E, LoBuglio A F, and Zinn K R, In vivo imaging for early detection and monitoring of human breast cancer in animal model during therapy. Eur J Nucl Med 28(8): 1000, 2001.
Chaudhuri T R, Rogers B E, Mountz J M, LoBuglio A F, and Zinn K R, Early detection and monitoring of breast tumor xenografts during therapy. Am Assoc of Cancer Res Proceedings Supplement:75, 2001.
Chaudhuri T R, Rogers B E, Mountz J M, Partridge E E, and Zinn K R, Non-invasive imaging of Ad-vector-transfected ovarian xenografts. Eur J Nucl Med 28(8):1172, 2001.
Chishima T, Miyagi Y, Li L, Tan Y, Baranov E, Yang M, Shimada H, Moossa A R, and Hoffman R M, Use of histoculture and green fluorescent protein to visualize tumor cell host interaction. In Vitro Cell Dev Biol Anim 33:745-7, 1997.
Chishima T, Miyagi Y, Wang X, Tan Y, Shimada H, Moossa A, and Hoffman R M, Visualization of the metastatic process by green fluorescent protein expression. Anticancer Res 17:2377-84, 1997.
Chishima T, Miyagi Y, Wang X, Yamaoka H, Shimada H, Moossa A R, and Hoffman R M, Cancer invasion and micrometastasis visualized in live tissue by green fluorescent protein expression. Cancer Res 57:2042-7, 1997.
Chishima T, Yang M, Miyagi Y, Li L, Tan Y, Baranov E, Shimada H, Moossa A R, Penman S, and Hoffman R M, Governing step of metastasis visualized in vitro. Proc Natl Acad Sci U S A 94:11573-6, 1997.
Chmiel J F, Berger M, and Konstan M W, The role of inflammation in the pathophysiology of CF lung disease. Clin Rev Allergy Immunol 23:5-27, 2002.
Chmiel J F, Konstan M W, and Berger M, Murine models of CF airway infection and inflammation. Methods Mol Med 70:495-515, 2002.
Cho J Y, Shen D H, Yang W, Williams B, Buckwalter T L, La Perle K M, Hinkle G, Pozderac R, Kloos R, Nagaraja H N, Barth R F, and Jhiang S M, In vivo imaging and radioiodine therapy following sodium iodide symporter gene transfer in animal model of intracerebral gliomas. Gene Ther 9:1139-45, 2002.
Christenson S D, Lake K D, Ooboshi H, Faraci F M, Davidson B L, and Heistad D D, Adenovirus-mediated gene transfer in vivo to cerebral blood vessels and perivascular tissue in mice. Stroke 29:1411-5; discussion 1416, 1998.
Chung J K, Sodium iodide symporter: its role in nuclear medicine. J Nucl Med 43:1188-200, 2002.
Cichon G, Boeckh-Herwig S, Schmidt H H, Wehnes E, Muller T, Pring-Akerblom P, and Burger R, Complement activation by recombinant adenoviruses. Gene Ther 8:1794-800, 2001.
Circolo A et al. Genetic disruption of the murine complement C3 promoter region generates deficient mice with extrahepatic expression of C3 mRNA. Immunopharmacology 1999; 42:135-149.
Contag C H, Jenkins D, Contag P R, and Negrin R S, Use of reporter genes for optical measurements of neoplastic disease in vivo. Neoplasia 2:41-52, 2000.
Contag P R, Olomu I N, Stevenson D K, and Contag C H, Bioluminescent indicators in living mammals. Nat Med 4:245-7, 1998.
Cope M B, Steele V E, Eto I, Juliana M M, Hill D L, and Grubbs C J, Prevention of methylnitrosourea-induced mammary cancers by 9-cis-retinoic acid and/or vitamin D3. Oncol Rep 9:533-7, 2002.
Cuntz M C, Levine E A, O'Dorisio T M, Watson J C, Wray D A, Espenan G D, McKnight C, Meier J R, Weber L J, Mera R, O'Dorisio M S, and Woltering E A, Intraoperative gamma detection of 125I-lanreotide in women with primary breast cancer. Ann Surg Oncol 6:367-72, 1999.
de Jong M, Breeman W A, Bernard B F, Bakker W H, Schaar M, van Gameren A, Bugaj J E, Erion J, Schmidt M, Srinivasan A, and Krenning E P, [177Lu-DOTA(0),Tyr3] octreotate for somatostatin receptor-targeted radionuclide therapy. Int J. Cancer 92:628-33., 2001.
Dechecchi M C, Tamanini A, Bonizzato A, Cabrini G. Heparan sulfate glycosaminoglycans are involved in adenovirus type 5 and 2-host cell interactions. Virology 2000; 268: 382-390.
Dmitriev I P, Kashentseva E A, and Curiel D T, Engineering of adenovirus vectors containing heterologous peptide sequences in the C terminus of capsid protein IX. J Virol 76:6893-9,2002.
Dong J-Y, Fico S, Wang D, Van Ginkel F W, Pascual D W, and Frizzell R A, Systemic analysis of repeated gene delivery into animal lungs with a recombinant adenovirus vector. Human Gene Therapy 7:319-331, 1996.
Doring G and Worlitzsch D, Inflammation in cystic fibrosis and its management. Paediatr Respir Rev 1:101-6, 2000.
Dubois R N, Abramson S B, Crofford L, Gupta R A, Simon L S, Van De Putte L B, and Lipsky P E, Cyclooxygenase in biology and disease. Faseb J 12:1063-73, 1998.
Duthy T G, Ormsby R J, Giannakis E, Ogunniyi A D, Stroeher U H, Paton J C, and Gordon D L, The human complement regulator factor H binds pneumococcal surface protein PspC via short consensus repeats 13 to 15. Infect Immun 70:5604-11, 2002.
Einfeld D A, Schroeder R, Roelvink P W, Lizonova A, King C R, Kovesdi I, and Wickham T J, Reducing the native tropism of adenovirus vectors requires removal of both CAR and integrin interactions. J Virol 75:11284-91, 2001.
Fernie-King B, Seilly D J, Davies A, Lachmann P J. Subversion of the innate immune response by micro-organisms. Ann Rheum Dis 2002; 61 Suppl 2: ii8-12.
Fernie-King B A, Seilly D J, Davies A, Lachmann P J. Streptococcal inhibitor of complement inhibits two additional components of the mucosal innate immune system: secretory leukocyte proteinase inhibitor and lysozyme. Infect Immun 2002; 70: 4908-4916.
Feuerbach D, Fehlmann D, Nunn C, Siehler S, Langenegger D, Bouhelal R, Seuwen K, and Hoyer D, Cloning, expression and pharmacological characterisation of the mouse somatostatin sst(5) receptor. Neuropharmacology 39:1451-62, 2000.
Fukuda M N, Ohyama C, Lowitz K, Matsuo O, Pasqualini R, Ruoslahti E, and Fukuda M, A peptide mimic of E-selectin ligand inhibits sialyl Lewis X-dependent lung colonization of tumor cells. Cancer Res 60:450-6, 2000.
Gambhir S S, Barrio J R, Herschman H P, and Phelps M E, Assays for noninvasive imaging of reporter gene expression. Nucl Med Biol 26:481-90, 1999.
Gambhir S S, Barrio J R, Phelps M E, Iyer M, Namavari M, Satyamurthy N, Wu L, Green L A, Bauer E, MacLaren D C, Nguyen K, Berk A J, Cherry S R, and Herschman H R, Imaging adenoviral-directed reporter gene expression in living animals with positron emission tomography. Proc Natl Acad Sci U S A 96:2333-8, 1999.
Gambhir S S, Barrio J R, Wu L, Iyer M, Namavari M, Satyamurthy N, Bauer E, Parrish C, MacLaren D C, Borghei A R, Green L A, Sharfstein S, Berk A J, Cherry S R, Phelps M E, and Herschman H R, Imaging of adenoviral-directed herpes simplex virus type 1 thymidine kinase reporter gene expression in mice with radiolabeled ganciclovir. J Nucl Med 39:2003-11,1998.
Gambhir S S, Bauer E, Black M E, Liang Q, Kokoris M S, Barrio J R, Iyer M, Namavari M, Phelps M E, and Herschman H R, A mutant herpes simplex virus type 1 thymidine kinase reporter gene shows improved sensitivity for imaging reporter gene expression with positron emission tomography. Proc Natl Acad Sci U S A 97:2785-90,2000.
Gambhir S S, Herschman H R, Cherry S R, Barrio J R, Satyamurthy N, Toyokuni T, Phelps M E, Larson S M, Balatoni J, Finn R, Sadelain M, Tjuvajev J, and Blasberg R, Imaging transgene expression with radionuclide imaging technologies. Neoplasia 2:118-38, 2000.
Gomez-Navarro J, Krasnykh V N, Liu B, Wang W, Barnes M N, Alvarez R D, Siegal G P, Zinn K R, and Curiel D T, Towards clinical exploitation of novel vector paradigms: an adenoviral vector with enhanced infectivity and engineered capacity for in vivo imaging. Mol Ther 1(5):S178, 2000.
Greene G L, In vivo imaging reveals estrogen receptor's hidden personality. Nat Med 9:22-3, 2003.
Griscelli F, Opolon P, Saulnier P, Mami-Chouaib F, Gautier E, Echchalir H, Angevin E, Le Chevalier T, Bataille V, Squiban P, Tursz T, and Escudier B, Recombinant adenovirus shedding after intratumoral gene transfer in lung cancer patients. Gene Ther 10:386-95, 2003.
Groot-Wassink T, Aboagye E O, Glaser M, Lemoine N R, and Vassaux G, Adenovirus biodistribution and noninvasive imaging of gene expression in vivo by positron emission tomography using human sodium/iodide symporter as reporter gene. Hum Gene Ther 13:1723-35, 2002.
Grubbs C J, Farnell D R, Hill D L, and McDonough K C, Chemoprevention of N-nitroso-N-methylurea-induced mammary cancers by pretreatment with 17 beta-estradiol and progesterone. J Natl Cancer Inst 74:927-31, 1985.
Hakkarainen T, Hemminki A, Pereboev A V, Barker S D, Asiedu C K, Strong T V, Kanerva A, Wahlfors J, and Curiel D T, CD40 is expressed on ovarian cancer cells and can be utilized for targeting adenoviruses. Clin Cancer Res 9:619-24, 2003.
Hawk C and Leary S, Formulary of Laboratory Animals. 1995: Iowa State University Press.
Hemminki A, Belousova N, Zinn K R, Liu B, Wang M, Chaudhuri T R, Rogers B E, Buchsbaum D J, Siegal G P, Barnes M N, Gomez-Navarro J, Curiel D T, and Alvarez R D, An adenovirus with enhanced infectivity mediates molecular chemotherapy of ovarian cancer cells and allows imaging of gene expression. Mol Ther 4:223-31, 2001.
Hemminki A, Zinn K R, Bauerschmitz G J, Chaudhuri T R, Barnes M N, Alvarez R D, and Curiel D T, Integrin targeted adenoviruses for ovarian cancer gene therapy. Proc of ASCO 21a:82,2002.
Hemminki A, Zinn K R, Liu B, Chaudhuri T R, Desmond R A, Rogers B E, Barnes M N, Alvarez R D, and Curiel D T, In vivo molecular chemotherapy and noninvasive imaging with an infectivity-enhanced adenovirus. J Natl Cancer Inst 94:741-9, 2002.
Henze M, Schuhmacher J, Hipp P, Kowalski J, Becker D W, Doll J, Macke H R, Hofmann M, Debus J, and Haberkorn U, PET imaging of somatostatin receptors using [⁶⁸GA]DOTA-D-Phe¹-Tyr³-octreotide: first results in patients with meningiomas. J Nucl Med 42:1053-6, 2001.
Hofmann M, Maecke H, Borner A, Weckesser E, Schoffski P, Oei M L, Schumacher J, Henze M, Heppeler A, Meyer G J, and Knapp W H, Biokinetics and imaging with the somatostatin receptor PET radioligand ⁶8Ga-DOTATOC: preliminary data. Eur J Nucl Med 28:1751-1757,2001.
Hoyer D, Bell G L Berelowitz M, Epelbaum J, Feniuk W, Humphrey P P, O'Carroll A M, Patel Y C, Schonbrunn A, Taylor J E, and et al., Classification and nomenclature of somatostatin receptors. Trends Pharmacol Sci 16:86-8, 1995.
Hustinx R, Shiue C Y, Alavi A, McDonald D, Shiue G G, Zhuang H, Lanuti M, Lambright E, Karp J S, and Eck S L, Imaging in vivo herpes simplex virus thymidine kinase gene transfer to tumour-bearing rodents using positron emission tomography and. Eur J Nucl Med 28:5-12,2001.
Ikeda K et al. Oncolytic virus therapy of multiple tumors in the brain requires suppression of innate and elicited antiviral responses. Nat Med 1999; 5: 881-887.
Ikeda K, Wakimoto H, Ichikawa T, Jhung S, Hochberg F H, Louis D N, and Chiocca E A, Complement depletion facilitates the infection of multiple brain tumors by an intravascular, replication-conditional herpes simplex virus mutant. J Virol 74:4765-75, 2000.
Inoue H, Kosaka T, Miyata A, Hara S, Yokoyama C, Nanayama T, and Tanabe T, Structure and expression of the human prostaglandin endoperoxide synthase 2 gene. Adv Prostaglandin Thromboxane Leukot Res 23:109-11, 1995.
Inoue H, Yokoyama C, and Tanabe T, [Structure and expression of an inducible prostaglandin endoperoxide synthase gene]. Tanpakushitsu Kakusan Koso 40:399-408, 1995.
Inoue H, Yokoyama C, Hara S, Tone Y, and Tanabe T, Transcriptional regulation of human prostaglandin-endoperoxide synthase-2 gene by lipopolysaccharide and phorbol ester in vascular endothelial cells. Involvement of both nuclear factor for interleukin-6 expression site and cAMP response element. J Biol Chem 270:24965-71, 1995.
Iris B, Zilberman Y, Zeira E, Galun E, Honigman A, Turgeman G, Clemens T, Gazit Z, and Gazit D, Molecular imaging of the skeleton: quantitative real-time bioluminescence monitoring gene expression in bone repair and development. J Bone Miner Res 18:570-8, 2003.
Iyer M, Wu L, Carey M, Wang Y, Smallwood A, and Gambhir S S, Two-step transcriptional amplification as a method for imaging reporter gene expression using weak promoters. Proc Natl Acad Sci U S A 98:14595-14600, 2001.
Jacobs A, Tjuvajev J G, Dubrovin M, Akhurst T, Balatoni J, Beattie B, Joshi R, Finn R, Larson S M, Herrlinger U, Pechan P A, Chiocca E A, Breakefield X O, and Blasberg R G, Positron emission tomography-based imaging of transgene expression mediated by replication-conditional, oncolytic herpes simplex virus type 1 mutant vectors in vivo. Cancer Res 61:2983-95, 2001.
Jones H P, Hodge L M, Fujihashi K, Kiyono H, McGhee J R, and Simecka J W, The pulmonary environment promotes Th2 cell responses after nasal-pulmonary immunization with antigen alone, but Th1 responses are induced during instances of intense immune stimulation. J. Immunol. 167:4518-4526, 2001.
Jooss K, Chirmule N. Immunity to adenovirus and adeno-associated viral vectors: implications for gene therapy. Gene Ther 2003; 10: 955-963.
Kaufmann S H, Immunity to intracellular microbial pathogens. Immunol. Today 16:338-342, 1995.
Kent G, Iles R, Bear C E, Huan L J, Griesenbach U, McKerlie C, Frndova H, Ackerley C, Gosselin D, Radzioch D, O'Brodovich H, Tsui L C, Buchwald M, and Tanswell A K, Lung disease in mice with cystic fibrosis. J. Clin Invest 100:3060-9, 1997.
Kim M, Zinn K R, Barnett B G, Sumerel L A, Krasnykh V, Curiel D T, and Douglas J T, The therapeutic efficacy of adenoviral vectors for cancer gene therapy is limited by a low level of primary adenovirus receptors on tumour cells. Eur J. Cancer 38:1917-26, 2002.
Kirby I et al. Identification of contact residues and definition of the CAR-binding site of adenovirus type 5 fiber protein. J Virol 2000; 74: 2804-2813.
Kirby I et al. Mutations in the DG loop of adenovirus type 5 fiber knob protein abolish high-affinity binding to its cellular receptor CAR. J Virol 1999; 73: 9508-9514.
Kluth B, Hess S, Engelmann H, Schafnitzel S, Riethmuller G, and Feucht H E, Endothelial expression of CD40 in renal cell carcinoma. Cancer Res 57:891-9, 1997.
Kobayashi K, Matsumoto S, Morishima T, Kawabe T, and Okamoto T, Cimetidine inhibits cancer cell adhesion to endothelial cells and prevents metastasis by blocking E-selectin expression. Cancer Res 60:3978-84, 2000.
Koh E, Patrikeos A, Steens R, and Robins P, Hilar bronchial carcinoid tumor: increased peritumoral vascular expression of somatostatin-2 receptor on an octreotide study? Clin Nucl Med 26:870-1, 2001.
Kopf M, Gross G L, Bachmann M, Lamers M C, Bluethmann H, and Kohler G, Disruption of the murine IL-4 gene blocks Th2 cytokine responses. Nature 362:245-248, 1993.
Kraling B M, Razon M J, Boon L M, Zurakowski D, Seachord C, Darveau R P, Mulliken J B, Corless C L, and Bischoff J, E-selectin is present in proliferating endothelial cells in human hemangiomas. Am J. Pathol 148:1181-91, 1996.
Krasnykh V, Belousova N, Korokhov N, Mikheeva G, and Curiel D T, Genetic targeting of an adenovirus vector via replacement of the fiber protein with the phage T4 fibritin. J Virol 75:4176-83, 2001.
Krasnykh V, Dmitriev I, Mikheeva G, Miller C R, Belousova N, and Curiel D T, Characterization of an adenovirus vector containing a heterologous peptide epitope in the HI loop of the fiber knob. J Virol 72:1844-52, 1998.
Krasnykh V, Dmitriev I, Navarro J G, Belousova N, Kashentseva E, Xiang J, Douglas J T, and Curiel D T, Advanced generation adenoviral vectors possess augmented gene transfer efficiency based upon coxsackie adenovirus receptor-independent cellular entry capacity. Cancer Res 60:6784-7, 2000.
Krasnykh V N, Mikheeva G V, Douglas J T, and Curiel D T, Generation of recombinant adenovirus vectors with modified fibers for altering viral tropism. J Virol 70:6839-46, 1996.
Lachmann P J. Microbial subversion of the immune response. Proc Natl Acad Sci USA 2002; 99: 8461-8462.
Langley R R, Russell J, Eppihimer M J, Alexander S J, Gerritsen M, Specian R D, and Granger D N, Quantification of murine endothelial cell adhesion molecules in solid tumors. Am J. Physiol 277:H1156-66, 1999.
Larsen S K, Solomon H F, Caldwell G, and Abrams M J, [99mTc]tricine: a useful precursor complex for the radiolabeling of hydrazinonicotinate protein conjugates. Bioconjug Chem 6:635-8, 1995.
Lerondel S, Le Pape A, Sene C, Faure L, Bernard S, Diot P, Nicolis E, Mehtali M, Lusky M, Cabrini G, and Pavirani A, Radioisotopic imaging allows optimization of adenovirus lung deposition for cystic fibrosis gene therapy. Hum Gene Ther 12:1-11, 2001.
Li C Y, Shan S, Huang Q, Braun R D, Lanzen J, Hu K, Lin P, and Dewhirst M W, Initial stages of tumor cell-induced angiogenesis: evaluation via skin window chambers in rodent models. J Natl Cancer Inst 92:143-7, 2000.
Liang Q, Satyamurthy N, Barrio J R, Toyokuni T, Phelps M P, Gambhir S S, and Herschman H R, Noninvasive, quantitative imaging in living animals of a mutant dopamine D2 receptor reporter gene in which ligand binding is uncoupled from signal transduction. Gene Ther 8:1490-8, 2001.
Lipsky P E, Brooks P, Crofford L J, DuBois R, Graham D, Simon L S, van de Putte L B, and Abramson S B, Unresolved issues in the role of cyclooxygenase-2 in normal physiologic processes and disease. Arch Intern Med 160:913-20, 2000.
Lohr F, Huang Q, Hu K, Dewhirst M W, and Li C Y, Systemic vector leakage and transgene expression by intratumorally injected recombinant adenovirus vectors. Clin Cancer Res 7:3625-8, 2001.
Lowry O, Rosebrough N, Farr L, and Randall R, Protein measurement with the folin phenol reagent. J Biol Chem 193:265-275, 1951.
Lugtenburg P J, Krenning E P, Valkema R, Oei H Y, Lamberts S W, Eijkemans M J, van Putten W L, and Lowenberg B, Somatostatin receptor scintigraphy useful in stage I-II Hodgkin's disease: more extended disease identified. Br J Haematol 112:936-44, 2001.
MacLaren D C, Gambhir S S, Satyamurthy N, Barrio J R, Sharfstein S, Toyokuni T, Wu L, Berk A J, Cherry S R, Phelps M E, and Herschman H R, Repetitive, non-invasive imaging of the dopamine D2 receptor as a reporter gene in living animals. Gene Ther 6:785-91, 1999.
Maisch T, Kropff B, Sinzger C, and Mach M, Upregulation of CD40 expression on endothelial cells infected with human cytomegalovirus. J Virol 76:12803-12, 2002.
Manchanda R, Azure M, Lister-James J, Bush L, Zinn K, Baggs R, and Dean R, Tumor regression in rat pancreatic (AR42J) tumor-bearing mice with Re-188 P2045—a somatostatin analog. Clin Cancer Res 5:3769, 1999.
Marinaro M, Staats H F, Hiroi T, Coste M, Jackson R J, Boyaka P N, Okahashi N, Yamamoto M, Kiyono H, Bluethmann H, Fujihashi K, and McGhee J R, Mucosal adjuvant effect of cholera toxin in mice results from induction of T helper 2 (Th2) cells and IL-4. J. Immunol. 155:4621-4629, 1995.
Martens C L, Cwirla S E, Lee R Y, Whitehorn E, Chen E Y, Bakker A, Martin E L, Wagstrom C, Gopalan P, Smith C W, and et al., Peptides which bind to E-selectin and block neutrophil adhesion. J Biol Chem 270:21129-36, 1995.
Matsumoto S, Imaeda Y, Umemoto S, Kobayashi K, Suzuki H, and Okamoto T, Cimetidine increases survival of colorectal cancer patients with high levels of sialyl Lewis-X and sialyl Lewis-A epitope expression on tumour cells. Br J. Cancer 86:161-7, 2002.
McIntosh D P, Tan X Y, Oh P, and Schnitzer J E, Targeting endothelium and its dynamic caveolae for tissue-specific transcytosis in vivo: a pathway to overcome cell barriers to drug and gene delivery. Proc Natl Acad Sci U S A 99:1996-2001, 2002.
Meri T et al Onchocerca volvulus microfilariae avoid complement attack by direct binding of factor H. J Infect Dis 2002; 185: 1786-1793.
Meri T, Hartmann A, Lenk D, Eck R, Wurzner R, Hellwage J, Meri S, and Zipfel P F, The yeast Candida albicans binds complement regulators factor H and FHL-1. Infect Immun 70:5185-92, 2002.
Meri T, Jokiranta T S, Hellwage J, Bialonski A, Zipfel P F, and Meri S, Onchocerca volvulus microfilariae avoid complement attack by direct binding of factor H. J Infect Dis 185:1786-93, 2002.
Moffatt S, Hays J, HogenEsch H, and Mittal S K, Circumvention of vector-specific neutralizing antibody response by alternating use of human and non-human adenoviruses: implications in gene therapy. Virology 272:159-167, 2000.
Moore A, Josephson L, Bhorade R M, Basilion J P, and Weissleder R, Human transferrin receptor gene as a marker gene for mr imaging. Radiology 221:244-50, 2001.
Mosmann T R and Coffman R L, Th1 and Th2 cells: different patterns of lymphokine secretion lead to different functional properties. Annu. Rev. Immunol. 7:145-173, 1989.
Nakamura T, Sato K, Hamada H. Reduction of natural adenovirus tropism to the liver by both ablation of fiber-coxsackievirus and adenovirus receptor interaction and use of replaceable short fiber. J Virol 2003; 77: 2512-2521.
Nguyen H H, van Ginkel F W, Vu H L, McGhee J R, and Mestecky J, Heterosubtypic immunity to influenza A virus infection requires B cells but not cytotoxic T lymphocytes (CTL). J. Infect Dis. 183:368-376, 2001.
Nguyen M, Corless C L, Kraling B M, Tran C, Atha T, Bischoff J, and Barsky S H, Vascular expression of E-selectin is increased in estrogen-receptor-negative breast cancer: a role for tumor-cell-secreted interleukin-1 alpha. Am J. Pathol 150:1307-14, 1997.
Ogawa O, Umegaki H, Ishiwata K, Asai Y, Ikari H, Oda K, Toyama H, Ingram D K, Roth G S, Iguchi A, and Senda M, In vivo imaging of adenovirus-mediated over-expression of dopamine D2 receptors in rat striatum by positron emission tomography. Neuroreport 11:743-8, 2000.
Ohtsuki K, Akashi K, Aoka Y, Blankenberg F G, Kopiwoda S, Tait J F, and Strauss H W, Technetium-99m HYNIC-annexin V: a potential radiopharmaceutical for the in-vivo detection of apoptosis. Eur J Nucl Med 26:1251-8, 1999.
Oikonomou A, Manavis J, Karagianni P, Tsanakas J, Wells A U, Hansell D M, Papadopoulou F, and Efremidis S C, Loss of FEV1 in cystic fibrosis: correlation with HRCT features. Eur Radiol 12:2229-35, 2002.
Pammer J, Plettenberg A, Weninger W, Diller B, Mildner M, Uthman A, Issing W, Sturzl M, and Tschachler E, CD40 antigen is expressed by endothelial cells and tumor cells in Kaposi's sarcoma. Am J Pathol 148:1387-96, 1996.
Pammer J, Weninger W, Mazal P R, Horvat R, and Tschachler E, Expression of the CD40 antigen on normal endothelial cells and in benign and malignant tumours of vascular origin. Histopathology 29:517-24, 1996.
Pascual D W, McGhee J R, Kiyono H, and Bost K L, Neuroimmune modulation of lymphocyte function. I. Substance P enhances immunoglobulin synthesis in lipopolysaccharide activated murine splenic B cell cultures. Int. Immunol. 3:1223-1229, 1991.
Ponnazhagan S, Curiel D T, Shaw D R, Alvarez R D, and Siegal G P, Adeno-associated virus for cancer gene therapy. Cancer Res 61:6313-21, 2001.
Ponnazhagan S, Mahendra G, Kumar S, Thompson J A, and Castillas M, Jr., Conjugate-based targeting of recombinant adeno-associated virus type 2 vectors by using avidin-linked ligands. J Virol 76:12900-7, 2002.
Quigg R J and Holers V M, Characterization of rat complement receptors and regulatory proteins. CR2 and Crry are conserved, and the C3b receptor of neutrophils and platelets is distinct from CR1. J Immunol 155:1481-8, 1995.
Ratjen F and Doring G, Cystic fibrosis. Lancet 361:681-9, 2003.
Ray P, Bauer E, Iyer M, Barrio J R, Satyamurthy N, Phelps M E, Herschman H R, and Gambhir S S, Monitoring gene therapy with reporter gene imaging. Semin Nucl Med 31:312-20, 2001.
Rennen H J, Boerman O C, Koenders E B, Oyen W J, and Corstens F H, Labeling proteins with Tc-99m via hydrazinonicotinamide (HYNIC): optimization of the conjugation reaction. Nucl Med Biol 27:599-604, 2000.
Reubi J C, Schar J C, Waser B, Wenger S, Heppeler A, Schmitt J S, and Macke H R, Affinity profiles for human somatostatin receptor subtypes SST1-SST5 of somatostatin radiotracers selected for scintigraphic and radiotherapeutic use. Eur J Nucl Med 27:273-82, 2000.
Reynolds P N, Zinn K R, Gavrilyuk V D, Balyasnikova I V, Rogers B E, Buchsbaum D J, Wang M H, Miletich D J, Grizzle W E, Douglas J T, Danilov S M, and Curiel D T, A targetable, injectable adenoviral vector for selective gene delivery to pulmonary endothelium in vivo. Mol Ther 2:562-78, 2000.
Rodriguez-Burford C, Lubet R A, Eto I, Juliana M M, Kelloff G J, Grubbs C J, and Steele V E, Effect of reduced body weight gain on the evaluation of chemopreventive agents in the methylnitrosourea-induced mammary cancer model. Carcinogenesis 20:71-6, 1999.
Rodriguez-Burford C, Lubet R A, Steele V E, Eto I, Bandy M, Juliana M M, Weiss H L, Grizzle W E, Kelloff G J, and Grubbs C J, Effects of acute and chronic body weight gain reductions in the evaluation of agents for efficacy in mammary cancer prevention. Oncol Rep 8:373-9, 2001.
Rogers B E, Chaudhuri T R, Reynolds P N, Della Manna D L, and Zinn K R, Non-Invasive gamma camera imaging of gene transfer using an adenoviral vector encoding an epitope-tagged receptor as a reporter. Gene Ther 10:105-114, 2003.
Rogers B E, Chaudhuri T R, Belousova N, Kirkman R L, Della Manna D, Krasnykh V N, and Zinn K R, Evaluation of a dual gene adenovirus encoding somatostatin receptor subtype 2 and cytosine deaminase in the context of human prostate cancer. Proc Am Assoc Cancer Res 43:84, 2002.
Rogers B E, Chaudhuri T R, Della Manna D, Robinson G D, and Zinn K, Evaluation of an adenoviral vector encoding a model chimeric receptor as a reporter for imaging gene transfer. Mol Ther 3:S57, 2001.
Rogers B E, Chaudhuri T R, Della Manna D L, Leay L L, Yamamoto M, and Zinn K R, Evaluation of an Adenvorial Vector Encoding Somatostatin Receptor Subtype 2 under the Control of a Cyclooxygenase-2 Promoter in the Context of Human Prostate Cancer. Mol Ther 5:S109, 2002.
Rogers B E, Zinn K R, and Buchsbaum D J, Gene transfer strategies for improving radiolabeled peptide imaging and therapy. Q J Nucl Med 44:208-23, 2000.
Rogers B E, Zinn K R, Chaudhuri T R, Lin C Y, and Buchsbaum D J, Targeted radiotherapy with Y-90-SMT 487 in mice bearing human non-small cell lung tumor xenografts induced to express human somatostatin receptor subtype 2 with an adenoviral vector. Cancer Biother Radiopharm 15:403, 2000.
Rogers B E, Zinn K R, Lin C Y, Chaudhuri T R, and Buchsbaum D J, Targeted radiotherapy with [(90)Y]-SMT 487 in mice bearing human non-small cell lung tumor xenografts induced to express human somatostatin receptor subtype 2 with an adenoviral vector. Cancer 94:1298-305, 2002.
Ruiz F E, Clancy J P, Perricone M A, Bebok Z, Hong J S, Cheng S H, Meeker D P, Young K R, Schoumacher R A, Weatherly M R, Wing L, Morris J E, Sindel L, Rosenberg M, Van Ginkel F W, McGhee J R, Kelly D, Lyrene R K, and Sorscher E J, A clinical inflammatory syndrome attributable to aerosolized lipid-DNA administration in cystic fibrosis. Human Gene Therapy 12:751-761, 2001.
Sagel S D, Sontag M K, Wagener J S, Kapsner R K, Osberg I, and Accurso F J, Induced sputum inflammatory measures correlate with lung function in children with cystic fibrosis. J. Pediatr 141:811-7, 2002.
Santis G et al. Molecular determinants of adenovirus serotype 5 fibre binding to its cellular receptor CAR. J Gen Virol 1999; 80 (Pt 6): 1519-1527.
Schellingerhout D, Bogdanov A, Jr., Marecos E, Spear M, Breakefield X, and Weissleder R, Mapping the in vivo distribution of herpes simplex virions. Hum Gene Ther 9:1543-9, 1998.
Schiffelers R M, Molema G, ten Hagen T L, Janssen A P, Schraa A J, Kok R J, Koning G A, and Storm G, Ligand-targeted liposomes directed against pathological vasculature. J Liposome Res 12:129-35, 2002.
Schnitzer J E, Caveolae: from basic trafficking mechanisms to targeting transcytosis for tissue-specific drug and gene delivery in vivo. Adv Drug Deliv Rev 49:265-80, 2001.
Schraa A J, Everts M, Kok R J, Asgeirsdottir S A, Meijer D K, de Leij L F, and Molema G, Development of vasculature targeting strategies for the treatment of cancer and chronic inflammatory diseases. Biotechnol Annu Rev 8:133-65, 2002.
Schraa A J, Kok R J, Moorlag H E, Bos E J, Proost J H, Meijer D K, de Leij L F, and Molema G, Targeting of RGD-modified proteins to tumor vasculature: a pharmacokinetic and cellular distribution study. Int J. Cancer 102:469-75, 2002.
Shayakhmetov D M et al. Binding of adenovirus fiber knob to blood coagulation factors mediates CAR-independent liver tropism. Mol Ther 2003; 7: S165.
Simecka J W, Patel P, Davis J K, Ross S E, Otwell P, and Cassell G H, Specific and nonspecific antibody responses in different segments of the respiratory tract in rats infected with Mycoplasma pulmonis. Infect. Immun. 59:3715-3721, 1991.
Simecka J W, Thorp R B, and Cassell G H, Dendritic cells are present in the alveolar region of lungs from specific pathogen-free rats. Reg. Immunol. 4:18-24, 1992.
Slooter G D, Mearadji A, Breeman W A, Marquet R L, de Jong M, Krenning E P, and van Eijck C H, Somatostatin receptor imaging, therapy and new strategies in patients with neuroendocrine tumours. Br J Surg 88:31-40, 2001.
Smith T A, Idamakanti N, Wright P M, Marshall-Neff J, Rollence M L, Nemerow G R, Mech C, Pinkstaff A, Kaloss M, Kaleko M, and Stevenson S C, Heparan sulfate proteoglycans, and not CAR or integrins, are the major receptors for hepatic adenoviral transduction in vivo. Mol Ther 5:S149, 2002.
Staal-van den Brekel A J, Thunnissen F B, Buurman W A, and Wouters E F, Expression of E-selectin, intercellular adhesion molecule (ICAM)-1 and vascular cell adhesion molecule (VCAM)-1 in non-small-cell lung carcinoma. Virchows Arch 428:21-7, 1996.
Statistical Analyses System (SAS), Release 8.0, SAS Institute Inc., Cary, N C.
Stegman L D, Rehemtulla A, Beattie B, Kievit E, Lawrence T S, Blasberg R G, Tjuvajev J G, and Ross B D, Noninvasive quantitation of cytosine deaminase transgene expression in human tumor xenografts with in vivo magnetic resonance spectroscopy. Proc Natl Acad Sci U S A 96:9821-6, 1999.
Stevens T L, Bossie A, Sanders V M, Fernandez-Botran R, Coffman R L, Mosmann T R, and Vitetta E S, Regulation of antibody isotype secretion by subsets of antigen-specific helper T cells. Nature 334:255-258, 1988.
Stoiber H, Speth C, Dierich M P. Role of complement in the control of HIV dynamics and pathogenesis. Vaccine 2003; 21 Suppl 2: S77-82.
Suresh M, Molina H, Salvato M S, Mastellos D, Lambris J D, and Sandor M, Complement component 3 is required for optimal expansion of CD8 T cells during a systemic viral infection. J Immunol 170:788-94,2003.
Szalai A J, Briles D E, and Volanakis J E, Role of complement in C-reactive-protein-mediated protection of mice from Streptococcus pneumoniae. Infect Immun 64:4850-3, 1996.
Tao N et al. Sequestration of adenoviral vector by Kupffer cells leads to a nonlinear dose response of transduction in liver. Mol Ther 2001; 3: 28-35.
Tatterson L E, Poschet J F, Firoved A, Skidmore J, and Deretic V, CFTR and pseudomonas infections in cystic fibrosis. Front Biosci 6:D890-7, 2001.
Thomas C E, Schiedner G, Kochanek S, Castro M G, and Lowenstein P R, Preexisting antiadenoviral immunity is not a barrier to efficient and stable transduction of the brain, mediated by novel high-capacity adenovirus vectors. Human Gene Therapy 12:839-846, 2001.
Thomas G R, Costelloe E A, Lunn D P, Stacey K J, Delaney S J, Passey R, McGlin E C, McMorran B J, Ahadizadeh A, Geczy C L, Wainwright B J, and Hume D A, G551D cystic fibrosis mice exhibit abnormal regulation of inflammation in lungs and macrophages. J Immunol 164:3870-7, 2000.
Thomas W D, Smith M J, Si Z, and Hersey P, Expression of the co-stimulatory molecule CD40 on melanoma cells. Int J. Cancer 68:795-801, 1996.
Tiddens H A, Detecting early structural lung damage in cystic fibrosis. Pediatr Pulmonol 34:228-31, 2002.
Tjuvajev J G, Avril N, Oku T, Sasajima T, Miyagawa T, Joshi R, Safer M, Beattie B, DiResta G, Daghighian F, Augensen F, Koutcher J, Zweit J, Humm J, Larson S M, Finn R, and Blasberg R, Imaging herpes virus thymidine kinase gene transfer and expression by positron emission tomography. Cancer Res 58:4333-41, 1998.
Tjuvajev J G, Chen S H, Joshi A, Joshi R, Guo Z S, Balatoni J, Ballon D, Koutcher J, Finn R, Woo S L, and Blasberg R G, Imaging adenoviral-mediated herpes virus thymidine kinase gene transfer and expression in vivo. Cancer Res 59:5186-93, 1999.
Tjuvajev J G, Finn R, Watanabe K, Joshi R, Oku T, Kennedy J, Beattie B, Koutcher J, Larson S, and Blasberg R G, Noninvasive imaging of herpes virus thymidine kinase gene transfer and expression: a potential method for monitoring clinical gene therapy. Cancer Res 56:4087-95, 1996.
Tjuvajev J G, Joshi A, Callegari J, Lindsley L, Joshi R, Balatoni J, Finn R, Larson S M, Sadelain M, and Blasberg R G, A general approach to the non-invasive imaging of transgenes using cis-linked herpes simplex virus thymidine kinase. Neoplasia 1:315-20, 1999.
Tong A W and Stone M J, Prospects for CD40-directed experimental therapy of human cancer. Cancer Gene Ther 10:1-13, 2003.
Tong A W, Papayoti M H, Netto G, Armstrong D T, Ordonez G, Lawson J M, and Stone M J, Growth-inhibitory effects of CD40 ligand (CD154) and its endogenous expression in human breast cancer. Clin Cancer Res 7:691-703, 2001.
Ultee M E, Bridger G J, Abrams M J, Longley C B, Burton C A, Larsen S K, Henson G W, Padmanabhan S, Gaul F E, and Schwartz D A, Tumor imaging with technetium-99m-labeled hydrazinonicotinamide-Fab′ conjugates. J Nucl Med 38:133-8, 1997.
Uotani H, Yamashita I, Nagata T, Kishimoto H, Kashii Y, and Tsukada K, Induction of E-selectin after partial hepatectomy promotes metastases to liver in mice. J Surg Res 96:197-203, 2001.
van Eijck C H, de Jong M, Breeman W A, Slooter G D, Marquet R L, and Krenning E P, Somatostatin receptor imaging and therapy of pancreatic endocrine tumors. Ann Oncol 10:177-81, 1999.
van Ginkel F W, Liu C, Simecka J W, Dong J-Y, Greenway T, Frizzell R A, Kiyono H, McGhee J R, and Pascual D W, Intratracheal gene delivery with adenoviral vector induces elevated systemic IgG and mucosal IgA antibodies to adenovirus and b-galactosidase. Hum Gene Ther 6:895-903, 1995.
van Ginkel F W, McGhee J R, Liu C, Simecka J W, Yamamoto M, Frizzell R A, Sorscher E J, Kiyono H, and Pascual D W, Adenoviral gene delivery elicits distinct pulmonary-associated T helper cell responses to the vector and to its transgene. J. Immunol. 159:685-693, 1997.
van Ginkel F W, Wahl S M, Kearney J F, Kweon M N, Fujihashi K, Burrows P D, Kiyono H, and McGhee J R, IgA-deficiency with increased Th2-type cytokines in TGF-beta 1 knockout mice. J. Immunol. 163:1951-1957, 1999.
Vanderkwaak T J et al An advanced generation of adenoviral vectors selectively enhances gene transfer for ovarian cancer gene therapy approaches. Gynecol Oncol 1999; 74: 227-234.
Virgolini I, Leimer M, Handmaker H, Lastoria S, Bischof C, Muto P, Pangerl T, Gludovacz D, Peck-Radosavljevic M, Lister-James J, Hamilton G, Kaserer K, Valent P, and Dean R, Somatostatin receptor subtype specificity and in vivo binding of a novel tumor tracer, 99mTc-P829. Cancer Res 58:1850-9, 1998.
Von Seggern D J, Huang S, Fleck S K, Stevenson S C, and Nemerow G R, Adenovirus vector pseudotyping in fiber-expressing cell lines: improved transduction of Epstein-Barr virus-transformed B cells. J Virol 74:354-62, 2000.
Vonderheide R H, Dutcher J P, Anderson J E, Eckhardt S G, Stephans K F, Razvillas B, Garl S, Butine M D, Perry V P, Armitage R J, Ghalie R, Caron D A, and Gribben J G, Phase I study of recombinant human CD40 ligand in cancer patients. J. Clin Oncol 19:3280-7, 2001.
Waibel R, Alberto R, Willuda J, Finnern R, Schibli R, Stichelberger A, Egli A, Abram U, Mach J P, Pluckthun A, and Schubiger P A, Stable one-step technetium-99m labeling of His-tagged recombinant proteins with a novel Tc(I)-carbonyl complex. Nat Biotechnol 17:897-901, 1999.
Walport M J. Complement. First of two parts. N Engl J Med 2001; 344: 1058-1066.
Walport M J. Complement. Second of two parts. N Engl J Med 2001; 344: 1140-1144.
Walter G, Barton E R, and Sweeney H L, Noninvasive measurement of gene expression in skeletal muscle. Proc Natl Acad Sci U S A 97:5151-5, 2000.
Watson J C, Balster D A, Gebhardt B M, O'Dorisio T M, O'Dorisio M S, Espenan G D, Drouant G J, and Woltering E A, Growing vascular endothelial cells express somatostatin subtype 2 receptors. Br J. Cancer 85:266-72, 2001.
Weiss H L, Rodriguez-Burford C, Grubbs C J, and Grizzle W E, Longitudinal analysis of mammary cancer multiplicity in chemoprevention studies. Anticancer Res 20:2281-7, 2000.
Wright M, Myles C, and Jackson R, Intratracheal “naked” DNA vaccination with pcDNA3-TNF-a, LPS-induced lung injury and mortality. Amer J Res Crit Care Med 165:A41, 2002.
Wu J C, Inubushi M, Sundaresan G, Schelbert H R, and Gambhir S S, Optical imaging of cardiac reporter gene expression in living rats. Circulation 105:1631-4, 2002.
Wu J C, Sundaresan G, Iyer M, and Gambhir S S, Noninvasive optical imaging of firefly luciferase reporter gene expression in skeletal muscles of living mice. Mol Ther 4:297-306, 2001.
Xu Y, Clark J C, Aronow B J, Dey C R, Liu C, Wooldridge J L, and Whitsett J A, Transcriptional adaptation to cystic fibrosis transmembrane conductance regulator deficiency. J Biol Chem 278:7674-82, 2003.
Xu-Amano J, Kiyono H, Jackson R J, Staats H F, Fujihashi K, Burrows P D, Elson C O, Pillai S, and McGhee J R, Helper T cell subsets for immunoglobulin A responses: oral immunization with tetanus toxoid and cholera toxin as adjuvant selectively induces Th2 in mucosa associated tissues. J. Exp. Med. 178:1309-1320, 1993.
Yamada Y, Post S R, Wang K, Tager H S, Bell G I, and Seino S, Cloning and functional characterization of a family of human and mouse somatostatin receptors expressed in brain, gastrointestinal tract, and kidney. Proc Natl Acad Sci U S A 89:251-5, 1992.
Yamamoto M, Alemany R, Adachi Y, Grizzle W E, and Curiel D T, Characterization of the cyclooxygenase-2 promoter in an adenoviral vector and its application for the mitigation of toxicity in suicide gene therapy of gastrointestinal cancers. Mol Ther 3:385-94, 2001.
Yang M, Baranov E, Jiang P, Sun F X, Li X M, Li L, Hasegawa S, Bouvet M, Al-Tuwaijri M, Chishima T, Shimada H, Moossa A R, Penman S, and Hoffman R M, Whole-body optical imaging of green fluorescent protein-expressing tumors and metastases. Proc Natl Acad Sci U S A 97:1206-11, 2000.
Yang M, Baranov E, Li X M, Wang J W, Jiang P, Li L, Moossa A R, Penman S, and Hoffman R M, Whole-body and intravital optical imaging of angiogenesis in orthotopically implanted tumors. Proc Natl Acad Sci U S A 98:2616-2621, 2001.
Yang M, Hasegawa S, Jiang P, Wang X, Tan Y, Chishima T, Shimada H, Moossa A R, and Hoffman R M, Widespread skeletal metastatic potential of human lung cancer revealed by green fluorescent protein expression. Cancer Res 58:4217-21, 1998.
Yang M, Jiang P, An Z, Baranov E, Li L, Hasegawa S, Al-Tuwaijri M, Chishima T, Shimada H, Moossa A R, and Hoffman R M, Genetically fluorescent melanoma bone and organ metastasis models. Clin Cancer Res 5:3549-59, 1999.
Yu H, Hanes M, Chrisp C E, Boucher J C, and Deretic V, Microbial pathogenesis in cystic fibrosis: pulmonary clearance of mucoid Pseudomonas aeruginosa and inflammation in a mouse model of repeated respiratory challenge. Infect Immun 66:280-8, 1998.
Yu Y, Annala A J, Barrio J R, Toyokuni T, Satyamurthy N, Namavari M, Cherry S R, Phelps M E, Herschman H R, and Gambhir S S, Quantification of target gene expression by imaging reporter gene expression in living animals. Nat Med 6:933-7, 2000.
Yuasa K, Sakamoto M, Miyagoe-Suzuki Y, Tanouchi A, Yamamoto H, Li J, Chamberlain J S, Xiao X, and Takeda S, Adeno-associated virus vector-mediated gene transfer into dystrophin-deficient skeletal muscles evokes enhanced immune response against the transgene product. Gene Therapy 9:1576-1588, 2002.
Yull F E et al. Bioluminescent detection of endotoxin effects on HIV-1 LTR-driven transcription in vivo. J Histochem Cytochem 2003; 51: 741-749.
Zaiss A K et al. Differential activation of innate immune responses by adenovirus and adeno-associated virus vectors. J Virol 2002; 76: 45804590.
Zinn K, Buchsbaum D, Chaudhuri T, Mountz J, Krasnykh V, Curiel D, and Rogers B, Simultaneous in vivo imaging of thymidine kinase and somatostatin receptor expression after gene transfer with an adenoviral vector encoding both genes. Mol Ther 1:S44, 2000.
Zinn K, Bush L, Mountz J, Chaudhuri T R, Hwang S, Dean R, and Azure M, Dose-dependent tumor suppression in nude mice by radiotherapy with Re-188-labeled P829, a somatostatin receptor (SSTR) avid peptide. J Nucl Med 5:223P, 1999.
Zinn K R and Chaudhuri T R, Imaging Adenovirus-Mediated Gene Transfer., in Adenoviral Vectors for Gene Therapy, Curiel D T and Douglas J T, Editors. 2002, Academic Press.
Zinn K R and Chaudhuri T R, The type 2 human somatostatin receptor as a platform for reporter gene imaging. Eur J Nucl Med 29:388-99, 2002.
Zinn K R, Buchsbaum D J, Chaudhuri T R, Mountz J M, and Rogers B E, Tc-99m-P2045: A new peptide for imaging somatostatin receptor gene transfer. J Nucl Med 41:81P, 2000.
Zinn K R, Buchsbaum D J, Chaudhuri T R, Mountz J M, Grizzle W E, and Rogers B E, Noninvasive monitoring of gene transfer using a reporter receptor imaged with a high-affinity peptide radiolabeled with 99mTc or 188Re. J Nucl Med 41:887-95, 2000.
Zinn K R, Cao Z, Ma Z, and Chaudhuri T R, Detection of Ovarian Cancer with a New Plasmid Vector Encoding Two Reporters: Blood-Based Screening Combined with Imaging. Mol Ther 5:S77, 2002.
Zinn K R, Cao Z, Partridge E E, and Chaudhuri T R, Noninvasive detection of ovarian cancer by simultaneous light-based and gamma-camera imaging. Proc of ASCO 21a:2994, 2002.
Zinn K R, Cao Z, Rodriguez-Burford C, and Chaudhuri T R, Validation of non-invasive light-based imaging by gamma camera imaging in prostate cancer xenografts. Eur J Nucl Med Mol Imaging 29:S163, 2002.
Zinn K R, Chaudhuri T R, Cao Z, Kastis G A, Bettan M, Liu Z, Howison C M, Stevenson G D, Furenlid L R, Wilson D W, and Barrett H H, Ultra-high resolution SPECT detects areas of Ad-mediated human type 2 somastotin receptor (hSSTr2) expression within xenograft tumors in live mice. Mol. Imag. Biol 4:S43,2002.
Zinn K R, Chaudhuri T R, Belousova N, Buchsbaum D J, Mountz J M, Curiel D T, Krasnykh M N, and Rogers B E, Dose-response comparison for two in vivo reporter genes for imaging transfer. Mol Ther 3:S29, 2001.
Zinn K R, Chaudhuri T R, Belousova N, Davis A, Mountz J J, Mountz J, Curiel D, and Krasnykh V, In vitro and in vivo imaging of 99mTc-labeled recombinant adenovirus. Mol Ther 3:S136, 2001.
Zinn K R, Chaudhuri T R, Belousova N, Davis A J, Curiel D T, and Krasnykh V N, Imaging evaluation of Tc-99m-labeled recombinant adenovirus. Eur J Nucl Med 28(8):1027, 2001.
Zinn K R, Chaudhuri T R, Belousova N, Davis A J, Mountz J D, Mountz J M, Curiel D T, and Krasnykh V N, In Vitro and In Vivo Imaging of 99mTc-Labeled Recombinant Adenovirus. Mol Ther 3:S136, 2001.
Zinn K R, Chaudhuri T R, Buchsbaum D, Mountz J, and Rogers B, Development of a non-invasive reporter system to image adenoviral-mediated gene transfer to ovarian cancer. Gynec Oncol 80:281, 2001.
Zinn K R, Chaudhuri T R, Buchsbaum D J, Mountz J M, and Rogers B E, A high capacity imaging method for detecting the expression of somatostatin receptor following gene transfer. J Nucl Med 41:81P, 2000.
Zinn K R, Chaudhuri T R, Buchsbaum D J, Mountz J M, and Rogers B E, Detection and measurement of in vitro gene transfer by gamma camera imaging. Gene Ther 8:291-9, 2001.
Zinn K R, Chaudhuri T R, Buchsbaum D J, Mountz J M, and Rogers B E, Simultaneous evaluation of dual gene transfer to adherent cells by gamma-ray imaging. Nucl Med Biol 28:135-44, 2001.
Zinn K R, Chaudhuri T R, Buchsbaum D J, Mountz J M, Curiel D T, Krasnykh V N, and Rogers B E, Dual In Vivo Imaging of Thymidine Kinase and Somatostatin Receptor Expression after Gene Transfer with a Bicistronic Adenoviral Vector. Eur J Nucl Med 27:1213, 2000.
Zinn K R, Chaudhuri T R, Krasnykh V N, Buchsbaum D J, Belousova N, Grizzle W E, Curiel D T, and Rogers B E, Gamma camera dual imaging with a somatostatin receptor and thymidine kinase after gene transfer with a bicistronic adenovirus in mice. Radiology 223:417-25, 2002.
Zinn K R, Chaudhuri T R, Krasnykh V N, Curiel D T, and Reynolds P N, In vivo imaging of Tc-99m-labeled ad vector redirected to lung following intravenous dosing. Eur J Nucl Med Mol Imaging 29:S107, 2002.
Zinn K R, Chaudhuri T R, Krasnykh V N, Curiel D T, and Reynolds R N, In Vivo Imaging Detects Tc-99m-Labeled Ad Redirected to Lung Following Intravenous Dosing. Mol Ther 5:S424,2002.
Zinn K R, Chaudhuri T R, Mountz J M, van den Berg G J, Gordon D T, and Johanning G L, 59Fe is retained from an elemental 59Fe powder supplement without effects on 65Zinc, 47Calcium and 67Copper in young pigs. J Nutr 129:181-7, 1999.
Zinn K R, Chaudhuri T R, Smyth C A, Wu Q, Liu H G, Fleck M, Mountz J D, and Mountz J M, Specific targeting of activated endothelium in rat adjuvant arthritis with a 99mTc-radiolabeled E-selectin-binding peptide. Arthritis Rheum 42:641-9, 1999.
Zinn K R, Chaudhuri T R, Stargel A V, Kumar S, and Ponnazhagan S, Gamma camera imaging of Tc-99m-labeled AAV vector followed by bioluminescence imaging of luciferase transgene expression. Mol Ther, Accepted (2003 ASGT annual meeting).
Zinn K R, Chaudhuri T R, Stargel A V, Kumar S, and Ponnazhagan S, Gamma camera imaging of Tc-99m-labeled AAV vector followed by bioluminescence imaging of luciferase transgene expression. Mol Ther, Submitted.
Zinn K R, Douglas J T, Smyth C A, Liu H G, Wu Q, Krasnykh V N, Mountz J D, Curiel D T, and Mountz J M, Imaging and tissue biodistribution of 99mTc-labeled adenovirus knob (serotype 5). Gene Ther 5:798-808, 1998.
Zinn K R, Kastis G A, Chaudhuri T R, Bettan M, Cao Z, Liu Z, Howison C M, Stevenson G D, Furenlid L R, Wilson D W, and Barrett H, Ultra-high Resolution SPECT Detects Areas of Ad-mediated Human Type 2 Somatostatin Receptor (hSSTr2) Expression within Xenograft Tumors in Live Mice. Abstract Book p. 29, Annual Investigators' Workshop, 2002.
Zinn K R, Kelpke S, Akhi K, Viera L, Chaudhuri T R, and Thompson J A, Glomerular targeting of acidic fibroblast growth factor-1 in renal transplanted rats1. Transplantation 73:1447-1453, 2002.
Zinn K R, Kelpke S, Chaudhuri T R, Sugg T, Mountz J M, and Thompson J A, Imaging Tc-99m-labeled FGF-1 targeting in rats. Nucl Med Biol 27:407-14, 2000.
Zinn K R, Krasnykh V N, Cao Z, and Chaudhuri T R, Dual genetic reporter system for early diagnosis and monitoring of breast cancer in mice. Breast Cancer Res. Treat. 76:S100, 2002.
Zinn K R, LoBuglio A F, Cao Z, and Chaudhuri T R, Non-invasive optical imaging of angiogenesis in breast cancer xenografts during therapy. Eur J Nucl Med Mol Imaging 29:S178, 2002.

What is claimed is

1. A method of detecting inflammation in a subject, comprising: (a) administering to said subject a vector, said vector comprising a reporter nucleic acid operably linked to a promoter nucleic acid, wherein said reporter nucleic acid is expressed under conditions of inflammation; and (b) detecting expression of said reporter nucleic acid by in vivo monitoring, expression of the reporter nucleic acid indicating inflammation.

2. The method of claim 1, wherein said vector is an adenovirus vector.

3. The method of claim 1, wherein said promoter nucleic acid is selected from the group consisting of a cox2L promoter and a cox2M promoter.

4. The method of claim 1, wherein the reporter nucleic acid encodes a light emitting protein.

5. The method of claim 4, wherein the light emitting protein is luciferase.

6. The method of claim 1, wherein the reporter nucleic acid encodes a fluorescent protein.

7. The method of claim 6, wherein the fluorescent protein is GFP.

8. The method of claim 6, wherein the fluorescent protein is RFP.

9. The method of claim 1, wherein the reporter nucleic acid encodes hSSTr2.

10. The method of claim 1, wherein the reporter nucleic acid is detectable by gamma ray imaging.

11. The method of claim 1, wherein the reporter nucleic acid encodes thymidine kinase (TK).

12. The method of claim 1, wherein the vector further comprises a complement modulator.

13. The method of claim 12, wherein the complement modulator inhibits complement activation.

14. The method of claim 13, wherein the complement inhibitor is SCR 13-15

15. The method of claim 13, wherein the complement inhibitor is Crry.

16. The method of claim 1, wherein expression of said reporter nucleic acid is detected by a labeled ligand for a polypeptide encoded by the reporter nucleic acid.

17. The method of claim 1, wherein said inflammation is associated with hepatitis.

18. The method of claim 1, wherein said inflammation is associated with lung inflammation.

19. The method of claim 1, wherein said inflammation is associated with an infectious process.

20. The method of claim 19, wherein the infectious process is a viral infection selected from the group consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian immunodeficiency cirus, Human T-cell Leukemia virus type-1, Hantavirus, Rubella virus, Simian Immunodeficiency virus, Human Immunodeficiency virus type-1, and Human Immunodeficiency virus type-2.

21. The method of claim 19, wherein the infectious process is a bacterial infection selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

22. The method of claim 19, wherein the infectious process is a parasitic infection selected from the group consisting of Toxoplasma gondii, Plasmodium, Trypanosoma brucei, Trypanosoma cruzi, Leishmania, Schistosoma, and Entamoeba histolytica.

23. The method of claim 19, wherein the infectious process is a fungal infection selected from the group consisting of Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternata.

24. The method of claim 1, wherein said inflammation is associated with liver toxicity.

25. The method of claim 24, wherein said liver toxicity is associated with cancer therapy.

26. The method of claim 25, wherein said cancer therapy is apoptosis induction.

27. The method of claim 25, wherein said cancer therapy is chemotherapy.

28. The method of claim 25, wherein said cancer therapy is a combination of chemotherapy and apoptosis induction.

29. The method of claim 1, wherein the inflammation is associated with an inflammatory disease.

30. The method of claim 29, wherein the inflammatory disease is selected from the group consisting of asthma, systemic lupus erythematosus, rheumatoid arthritis, reactive arthritis, spondyarthritis, systemic vasculitis, insulin dependent diabetes mellitus, multiple sclerosis, experimental allergic encephalomyelitis, Sjögren's syndrome, graft versus host disease, inflammatory bowel disease, ulcerative colitis, and scleroderma.

31. The method of claim 1, wherein the inflammation is associated with cancer.

32. The method of claim 31, wherein the cancer can be selected from the group consisting of lymphoma, leukemia, mycosis fungoide, carcinoma, adenocarcinoma, sarcoma, glioma, blastoma, neuroblastoma, plasmacytoma, histiocytoma, melanoma, adenoma, hypoxic tumour, myeloma, AIDS-related lymphoma or AIDS-related sarcoma, metastatic cancer, bladder cancer, brain cancer, nervous system cancer, glioblastoma, ovarian cancer, skin cancer, liver cancer, squamous cell carcinomas of the mouth, throat, larynx, and lung, colon cancer, cervical cancer, breast cancer, epithelial cancer, renal cancer, genitourinary cancer, pulmonary cancer, esophageal carcinoma, head and neck carcinoma, hematopoietic cancer, testicular cancer, colo-rectal cancer, prostatic cancer, and pancreatic cancer.

33. The method of claim 1, wherein the detection step comprises identifying activated cells at the site of inflammation.

34. The method of claim 1, wherein the inflammation is associated with transplant rejection.

35. The method of claim 1, wherein said in vivo monitoring is selected from the group consisting of bioluminescence imaging, gamma-ray irradiation, light-based imaging, magnetic resonance spectroscopy, and somatostatin receptor imaging.

36. The method of claim 1, wherein the vector further comprises a nucleic acid that encodes a detectable secreted protein.

37. The method of claim 36, wherein the detectable secreted protein is SEAP.

38. The method of claim 36, wherein expressing the reporter nucleic acid is detected by detecting the secreted protein.

39. The method of claim 1, wherein the subject is a transplant recipient.

40. A method of detecting inflammation in a transplant recipient comprising: (a) administering to cells of the transplant, prior to transplantation, a vector, said vector comprising a reporter nucleic acid and a promoter nucleic acid, wherein expression of said reporter nucleic acid is detectable under conditions of inflammation; (b) performing the transplant; and (c) detecting expression of said reporter nucleic acid by in vivo monitoring.

41. The method of claim 40, wherein said transplantation is organ transplantation.

42. The method of claim 41, wherein said organ transplantation is transplantation of the liver.

43. The method of claim 41, wherein said organ transplantation is transplantation of the kidney.

44. The method of claim 40, wherein said vector is an adenovirus vector.

45. The method of claim 40, wherein the vector further comprises a complement modulator.

46. The method of claim 45, wherein the complement modulator inhibits complement activation.

47. The method of claim 46, wherein the complement inhibitor is SCR 13-15.

48. The method of claim 46, wherein the complement inhibitor is Crry.

49. The method of claim 40, wherein said promoter nucleic acid is selected from the group consisting of a cox2L promoter and a cox2M promoter.

50. The method of claim 40, wherein the reporter nucleic acid encodes a light emitting protein.

51. The method of claim 50, wherein the light emitting protein is luciferase.

52. The method of claim 40, wherein the reporter nucleic acid encodes a fluorescent protein.

53. The method of claim 52, wherein the fluorescent protein is GFP.

54. The method of claim 52, wherein the fluorescent protein is RFP.

55. The method of claim 40, wherein the reporter nucleic acid encodes hSSTr2.

56. The method of claim 40, wherein the reporter nucleic acid is detectable by gamma ray imaging.

57. The method of claim 40, wherein the reporter nucleic acid encodes thymidine kinase (TK).

58. The method of claim 40, wherein expression of said reporter nucleic acid is detected by a labeled ligand for a polypeptide encoded by the reporter nucleic acid.

59. The method of claim 40, wherein said inflammation is associated with hepatitis.

60. The method of claim 40, wherein said inflammation is associated with lung inflammation.

61. The method of claim 40, wherein said inflammation is associated with an infectious process.

62. The method of claim 40, wherein the detection step comprises identifying activated cells at the site of inflammation.

63. The method of claim 40, wherein the inflammation is associated with transplant rejection.

64. The method of claim 40, wherein said in vivo monitoring is selected from the group consisting of bioluminescence imaging, gamma-ray irradiation, light-based imaging, magnetic resonance spectroscopy, and somatostatin receptor imaging.

65. The method of claim 40, wherein the vector further comprises a nucleic acid that encodes a detectable secreted protein.

66. The method of claim 65, wherein the detectable secreted protein is SEAP.

67. The method of claim 65, wherein expression of the reporter nucleic acid is detected by detecting the secreted protein.

68. A method of monitoring inflammation in a subject with an inflammatory or autoimmune disease, comprising: (a) administering to said subject a vector, said vector comprising a reporter nucleic acid operably linked to a promoter nucleic acid, wherein expression of said reporter nucleic acid is detectable under conditions of inflammation; and (b) detecting expression of said reporter nucleic acid by in vivo monitoring.

69. The method of claim 68, wherein the vector further comprises a complement modulator.

70. The method of claim 69, wherein the complement modulator inhibits complement activation.

71. The method of claim 70, wherein the complement inhibitor is SCR 13-15.

72. The method of claim 70, wherein the complement inhibitor is Crry.

73. The method of claim 68, wherein said vector is an adenovirus vector.

74. The method of claim 68, wherein said promoter nucleic acid is selected from the group consisting of a cox2L promoter and a cox2M promoter.

75. The method of claim 68, wherein the reporter nucleic acid encodes a light emitting protein.

76. The method of claim 75, wherein the light emitting protein is luciferase.

77. The method of claim 68, wherein the reporter nucleic acid encodes a fluorescent protein.

78. The method of claim 77, wherein the fluorescent protein is GFP.

79. The method of claim 77, wherein the fluorescent protein is RFP.

80. The method of claim 68, wherein the reporter nucleic acid encodes hSSTr2.

81. The method of claim 68, wherein the reporter nucleic acid is detectable by gamma ray imaging.

82. The method of claim 68, wherein the reporter nucleic acid encodes thymidine kinase (TK).

83. The method of claim 68, wherein expression of said reporter nucleic acid is detected by a labeled ligand for a polypeptide encoded by the reporter nucleic acid.

84. The method of claim 68, wherein the inflammatory disease can be selected from the group inflammatory diseases consisting of asthma, systemic lupus erythematosus, rheumatoid arthritis, reactive arthritis, spondyarthritis, systemic vasculitis, insulin dependent diabetes mellitus, multiple sclerosis, experimental allergic encephalomyelitis, Sjögren's syndrome, graft versus host disease, inflammatory bowel disease including Crohn's disease, ulcerative colitis, and scleroderma.

85. The method of claim 68, wherein the inflammation is associated with cancer.

86. The method of claim 85, wherein the cancer can be selected from the group consisting of lymphomas (Hodgkins and non-Hodgkins), B cell lymphoma, T cell lymphoma, myeloid leukemia, leukemias, mycosis fungoides, carcinomas, carcinomas of solid tissues, squamous cell carcinomas, adenocarcinomas, sarcomas, gliomas, blastomas, neuroblastomas, plasmacytomas, histiocytomas, melanomas, adenomas, hypoxic tumours, myelomas, AIDS-related lymphomas or sarcomas, metastatic cancers, bladder cancer, brain cancer, nervous system cancer, squamous cell carcinoma of head and neck, neuroblastoma/glioblastoma, ovarian cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, hematopoietic cancers, testicular cancer, colo-rectal cancers, prostatic cancer, or pancreatic cancer.

87. The method of claim 68, wherein the detection step comprises identifying activated cells at the site of inflammation.

88. The method of claim 68, wherein said in vivo monitoring is selected from the group consisting of bioluminescence imaging, gamma-ray irradiation, light-based imaging, magnetic resonance spectroscopy, and somatostatin receptor imaging.

89. The method of claim 68, wherein the vector further comprises a nucleic acid that encodes a detectable secreted protein.

90. The method of claim 89, wherein the detectable secreted protein is SEAP.

91. The method of claim 89, further comprising determining the activation status of the promoter by detecting the secreted protein.

92. A method of identifying a vector capable of detecting inflammation, comprising: (a) administering a vector to a cell culture, wherein the vector comprises a promoter nucleic acid and a reporter nucleic acid; (b) inducing an inflammatory response in said cell culture; and (c) monitoring expression of the reporter nucleic acid, expression indicating a vector capable of detecting inflammation.

93. The method of claim 92, wherein said promoter nucleic acid is selected from the group consisting of a cox2L promoter and a cox2M promoter.

94. The method of claim 92, wherein the reporter nucleic acid encodes a light emitting protein.

95. The method of claim 94, wherein the light emitting protein is luciferase.

96. The method of claim 92, wherein the reporter nucleic acid encodes a fluorescent protein.

97. The method of claim 96, wherein the fluorescent protein is GFP.

98. The method of claim 96, wherein the fluorescent protein is RFP.

99. The method of claim 92, wherein the reporter nucleic acid encodes hSSTr2.

100. The method of claim 92, wherein the reporter nucleic acid is detectable by gamma ray imaging.

101. The method of claim 92, wherein the reporter nucleic acid encodes thymidine kinase (TK).

102. The method of claim 92, wherein expression of said reporter nucleic acid is detected by a labeled ligand for a polypeptide encoded by the reporter nucleic acid.

103. The method of claim 92, wherein the vector further comprises a nucleic acid that encodes a detectable secreted protein.

104. The method of claim 103, wherein the detectable secreted protein is SEAP.

105. The method of claim 103, wherein expression of the reporter nucleic acid is detected by detecting the secreted protein.

106. A method of treating a subject with an inflammatory disease comprising: (a) administering to said subject a vector, said vector comprising a reporter nucleic acid operably linked to a promoter nucleic acid, wherein said reporter nucleic acid is expressed under conditions of inflammation; (b) detecting expression of said reporter nucleic acid by in vivo monitoring; and (c) modifying treatment of the subject when expression of said reporter nucleic acid is detected.

107. The method of claim 106, wherein said vector is an adenovirus vector.

108. The method of claim 106, wherein said promoter nucleic acid is selected from the group consisting of a cox2L promoter and a cox2M promoter.

109. The method of claim 106, wherein the reporter nucleic acid encodes a light emitting protein.

110. The method of claim 106, wherein the light emitting protein is luciferase.

111. The method of claim 106, wherein the reporter nucleic acid encodes a fluorescent protein.

112. The method of claim 111, wherein the fluorescent protein is GFP.

113. The method of claim 111, wherein the fluorescent protein is RFP.

114. The method of claim 106, wherein the reporter nucleic acid encodes hSSTr2.

115. The method of claim 106, wherein the reporter nucleic acid is detectable by gamma ray imaging.

116. The method of claim 106, wherein the vector further comprises a complement modulator.

117. The method of claim 116, wherein the complement modulator inhibits complement activation.

118. The method of claim 117, wherein the complement inhibitor is SCR 13-15.

119. The method of claim 117, wherein the complement inhibitor is Crry.

120. The method of claim 106, wherein the reporter nucleic acid encodes thymidine kinase (TK).

121. The method of claim 106, wherein expression of said reporter nucleic acid is detected by a labeled ligand for a polypeptide encoded by the reporter nucleic acid.

122. The method of claim 106, wherein the inflammation is associated with an inflammatory disease.

123. The method of claim 122, wherein the inflammatory disease can be selected from the group inflammatory diseases consisting of asthma, systemic lupus erythematosus, rheumatoid arthritis, reactive arthritis, spondyarthritis, systemic vasculitis, insulin dependent diabetes mellitus, multiple sclerosis, experimental allergic encephalomyelitis, Sjögren's syndrome, graft versus host disease, inflammatory bowel disease, ulcerative colitis, and scleroderma.

124. A method of reducing inflammation in a subject, comprising delivering to the subject a complement modulator.

125. The method of claim 124, wherein the complement modulator inhibits complement activation.

126. The method of claim 125, wherein the complement inhibitor is SCR 13-15.

127. The method of claim 126, wherein the complement inhibitor is Crry.

128. A transgenic animal, wherein the animal comprises a reporter nucleic acid operably linked to a promoter nucleic acid, wherein said reporter nucleic acid is expressed under conditions of inflammation.

129. A cell line comprising a vector, said vector comprising a reporter nucleic acid operably linked to a promoter nucleic acid, wherein said reporter nucleic acid is expressed under conditions of inflammation.

130. The method of claim 124, wherein a vector comprising SEQ ID NO: 8 is administered to the subject.

131. The method of claim 130, wherein the vector is inserted into HVR2 region of an adenovirus vector.

132. The method of claim 130, wherein the vector is inserted into HVR5 region of an adenovirus vector.

133. A nucleic acid comprising a nucleotide sequence that encodes at least two repeats of ED1 and a linker.

134. The method of claim 130, wherein the vector is inserted into any HVR region.

135. The nucleic acid sequence of claim 133, wherein the nucleotide sequence further encodes a His-tag.

136. The nucleic acid of claim 133, wherein the nucleotide sequence encodes SEQ ID NO: 9.

137. A nucleic acid comprising a nucleotide sequence at least 80% identical to of SEQ ID NO: 8.

138. The nucleic acid sequence of claim 137, wherein the nucleotide sequence at least 85% identical to of SEQ ID NO: 8.

139. The nucleic acid sequence of claim 137, wherein the nucleotide sequence at least 90% identical to of SEQ ID NO: 8.

140. The nucleic acid sequence of claim 137, wherein the nucleotide sequence at least 95% identical to of SEQ ID NO: 8.

141. A nucleic acid comprising the nucleotide sequence of SEQ ID NO: 8.

142. A vector comprising the nucleic acid of claim 133 operably linked to an expression control sequence.

143. The vector of claim 142, wherein the vector is a viral vector and wherein the nucleic acid is inserted in a hypervariable region of the viral genome.

144. The vector of claim 143, wherein the vector is an adenoviral vector.

145. The vector of claim 143, wherein the vector is an AAV.

146. A vector comprising the nucleic acid of claim 141 operably linked to an expression control sequence.

147. The vector of claim 146, wherein the vector is a viral vector and wherein the nucleic acid is inserted in a hypervariable region of the viral genome.

148. The vector of claim 147, wherein the vector is an adenoviral vector.

149. The vector of claim 147, wherein the vector is an AAV.

150. A polypeptide encoded by the nucleic acid sequence of claim 133.

151. A vector comprising the polypeptide of claim 150, wherein the polypeptide is on the vector surface.
